(19) United States
(12) Patent Application Publication
(10) Pub. No.: US 2021/0323981 A1
(43) Pub. Date: Oct. 21, 2021

(54) FREE AMINO COMPOUNDS FOR THE TREATMENT AND PROPHYLAXIS OF BACTERIAL INFECTION

(71) Applicant: Hoffmann-La Roche Inc., Little Falls, NJ (US)

(72) Inventors: Hong SHEN, Shanghai (CN); Xuefei TAN, Shanghai (CN); Chengang ZHOU, Shanghai (CN); Mingwei ZHOU, Shanghai (CN); Yimin HU, Shanghai (CN); Houguang SHI, Shanghai (CN); Fabian DEY, Basel (CH); Yongqiang LIU, Shanghai (CN); Xiao DING, Shanghai (CN)

(73) Assignee: Hoffmann-La Roche Inc., Little Falls, NJ (US)

(21) Appl. No.: 17/326,241

(22) Filed: May 20, 2021

Related U.S. Application Data
(63) Continuation of application No. PCT/EP2019/081760, filed on Nov. 19, 2019.

(30) Foreign Application Priority Data
Nov. 22, 2018 (CN) PCT/CN2018/116868

(51) Int. Cl. C07D 519/00

(52) U.S. Cl. CPC C07D 519/00

(57) ABSTRACT

The present invention relates to novel compounds of formula (I),

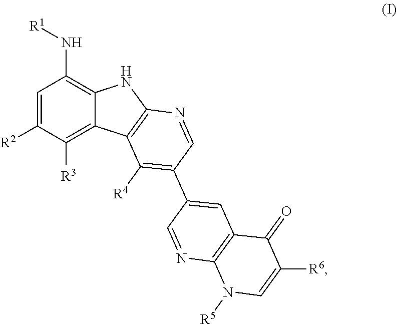

wherein R¹, R², R³, R⁴, R⁵ and R⁶ are as described herein, and their pharmaceutically acceptable salt, enantiomer or diastereomer thereof, and compositions including the compounds and methods of using the compounds.

### CROSS REFERENCE TO RELATED APPLICATIONS

This application is a continuation of International Application No. PCT/EP2019/081760 having an International filing date of Nov. 19, 2019, which claims benefit of and priority to International Application No. PCT/CN2018/116868 having an International filing date of Nov. 22, 2018, each of which are incorporated by reference in their entirety.

### BACKGROUND OF THE INVENTION

The present invention relates to organic compounds useful for therapy and/or prophylaxis in a mammal, and in particular to inhibitors of DNA gyrase and/or topoisomerase IV useful for treatment and/or prophylaxis of bacterial infection.

### FIELD OF THE INVENTION

Bacterial infections pose a continuing medical problem because anti-bacterial drugs eventually engender resistance in the bacteria on which they are used. Bacterial resistance against virtually all current antibiotic drugs are increasing. Many forms of antibiotic resistance can even cross international boundaries and spread with remarkable speed. Thus novel classes of antibacterial compounds are urgently needed.

One target for development of anti-bacterial drugs has been DNA gyrase and topoisomerase IV (bacterial type IIA topoisomerases), which are essential to cell life, that solve DNA topological problems resulting from the replication, transcription, and recombination of DNA. DNA Gyrase controls DNA supercoiling and relieves topological stress that occurs when the DNA strands are untwisted such as during replication. Topoisomerase IV primarily resolves linked chromosome dimers at the conclusion of DNA replication. Both enzymes can introduce double stranded DNA breaks; pass a second DNA strand through the break and rejoining the broken strands. The activity of both enzymes is driven by the binding and hydrolysis of ATP. Bacterial DNA gyrase consists of two A (GyrA) and two B (GyrB) subunits. Binding and cleavage of the DNA is associated with GyrA, whereas ATP is bound and hydrolyzed by GyrB. Bacterial Topoisomerase IV is also a hetero-tetramer that consists of two C (ParC) and two E (ParE) subunits. The latter subunits bind ATP like GyrB in order to supply energy necessary for catalytic turnover of the enzymes.

Inhibition of DNA gyrase and topoisomerase IV has potential for the development of broad-spectrum antibiotics. The enzymes are highly conserved across a broad range of gram-positive and gram-negative pathogens. There are two classes of antibiotics that demonstrated such mechanism of action. The first, well-represented by the quinolones, inhibits GyrA and ParC subunits by stabilizing the cleaved DNA-enzyme complex, thus inhibiting overall gyrase function, leading to cell death. Novobiocin, the only marketed drug in the second class, exerts its effect by blocking the ATPase activity of the enzymes. Novobiocin was identified in 1950s. But its use declined rapidly and it was eventually withdrawn from the market, mainly due to its low permeability in many bacteria strains, rise of spontaneous resistance development, and the development of more effective drugs, such as penicillinase-stable penicillins and the first cephalosporins in 1960s and 1970s.

Recently, strong inhibition of DNA gyrase and/or topoisomerase IV has been recognized to be important for low resistance development in bacterial strains treated by inhibitors of the enzymes. Inhibitors of bacterial DNA gyrase and/or topoisomerase IV with different mechanism of action compare to the widely used quinolones will exhibit minimal cross resistance, and will be potentially useful in combating quinolone resistance that has increased significantly in the past few years.

### SUMMARY OF THE INVENTION

The present invention relates to novel compounds of formula (I),

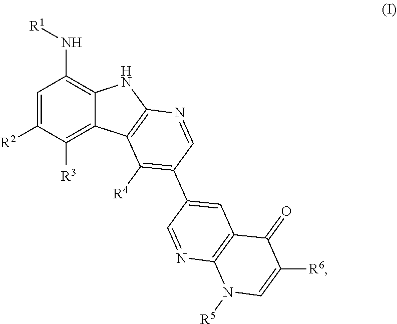

wherein
R¹ is C₁₋₆alkyl;
R² is halogen;
R³ is halogen or cyano;
R⁴ is 1,3,4,5,6,6a-hexahydrocyclopenta[c]pyrrolyl substituted by amino;
   2,3,3a,4,6,6a-hexahydropyrrolo[2,3-c]pyrrolyl substituted by amino;
   3,3a,4,5,6,6a-hexahydro-1H-cyclopenta[c]pyrrolyl substituted by amino;
   azabicyclo[3.1.0]hexanyl substituted by amino or aminoC₁₋₆alkyl;
   azaspiro[2.4]heptanyl substituted by amino;
   azaspiro[2.5]octanyl substituted by amino;
   azaspiro[3.3]heptanyl substituted by amino;
   piperidinyl substituted twice by aminoC₁₋₆alkyl and halogen;
   pyrrolidinyl substituted once or twice by substituents independently selected from amino, aminoC₁₋₆alkyl, aminoC₃₋₇cycloalkyl, haloC₁₋₆alkyl and halogen;
R⁵ is C₁₋₆alkyl;
R⁶ is carboxy;
or a pharmaceutically acceptable salt thereof.

Further objects of the present invention are novel compounds of formula (I), their manufacture, medicaments based on a compound in accordance with the invention and their production as well as the use of compounds of formula (I) for the treatment or prophylaxis of bacterial infection. The use of compounds of formula (I) as DNA gyrase and/or topoisomerase IV inhibitors is also one of the objections of present invention. The compounds of formula (I) showed superior anti-bacterial activity, good solubility, good CC₅₀ profiles, improved microsomal stability and/or improved PK profile.

### DETAILED DESCRIPTION OF THE INVENTION

Unless otherwise defined, all technical and scientific terms used herein have the same meaning as commonly understood by one of ordinary skill in the art to which this invention belongs. Furthermore, the following definitions are set forth to illustrate and define the meaning and scope of the various terms used to describe the invention.

### Definitions

The term “C₁₋₆alkyl” denotes a saturated, linear or branched chain alkyl group containing 1 to 6, particularly 1 to 4 carbon atoms, for example methyl, ethyl, propyl, isopropyl, n-butyl, isobutyl, tert-butyl and the like. Particular “C₁₋₆alkyl” groups are methyl, ethyl and propyl.

The term “halogen” and “halo” are used interchangeably herein and denote fluoro, chloro, bromo, or iodo.

The term “haloC₁₋₆alkyl” denotes an alkyl group wherein at least one of the hydrogen atoms of the alkyl group has been replaced by same or different halogen atoms, particularly fluoro atom. Examples of haloC₁₋₆alkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl, difluoromethyl, trifluoromethyl and trifluoroethyl.

The term “C₃₋₇cycloalkyl” denotes a saturated monocyclic or bicyclic carbon ring containing from 3 to 7 carbon atoms, particularly from 3 to 6 carbon atoms, for example, cyclopropyl, cyclobutyl, cyclopentyl, cyclohexyl, cycloheptyl, bicyclo[1.1.1]pentanyl and the like. Particular “C₃₋₇cycloalkyl” group is cyclopropyl.

The term “cis-isomers” and “trans-isomers” denote the relative stereochemistry of the molecule or moiety. For example: Intermediate C2

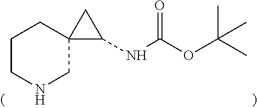

as the “cis-isomers” refers to a mixture of

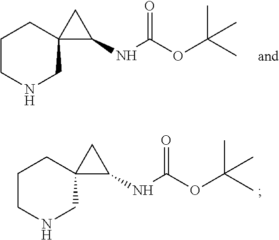 [SMILES: CC(C)(C)OC(=O)N[C@@H]1C[C@@]12CCCNC2 ; CC(C)(C)OC(=O)N[C@H]1C[C@]12CCCNC2]

similarly, Example 2.01

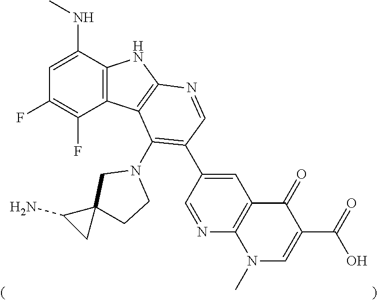

as the “trans-isomers” refers to a mixture of

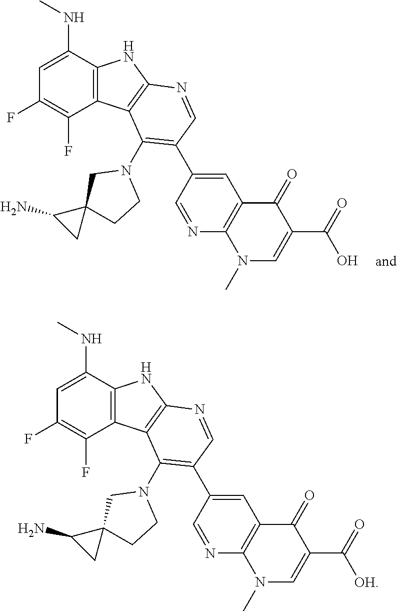 [SMILES: CNc1cc(F)c(F)c2c1[nH]c1ncc(-c3cnc4c(c3)c(=O)c(C(=O)O)cn4C)c(N3CC[C@@]4(C[C@@H]4N)C3)c12 ; CNc1cc(F)c(F)c2c1[nH]c1ncc(-c3cnc4c(c3)c(=O)c(C(=O)O)cn4C)c(N3CC[C@]4(C[C@H]4N)C3)c12]

In another embodiment, the term “rel” refers to relative configuration, for example, Example 1.13, 6-[5-Cyano-6-fluoro-8-(methylamino)-4-[rel-(3aR,4R,6aS)-4-amino-3,3a,4,5,6,6a-hexahydro-1H-cyclopenta[c]pyrrol-2-yl]-9H-pyrido[2,3-b]indol-3-yl]-1-methyl-4-oxo-1,8-naphthyridine-3-carboxylic acid

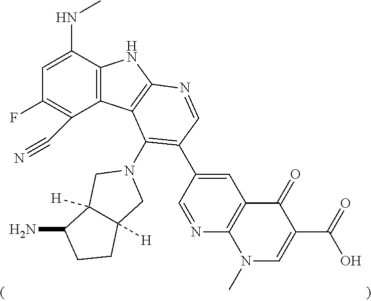 [SMILES: CNc1cc(F)c(C#N)c2c1[nH]c1ncc(-c3cnc4c(c3)c(=O)c(C(=O)O)cn4C)c(N3C[C@H]4CC[C@@H](N)[C@H]4C3)c12]

refers to a mixture of

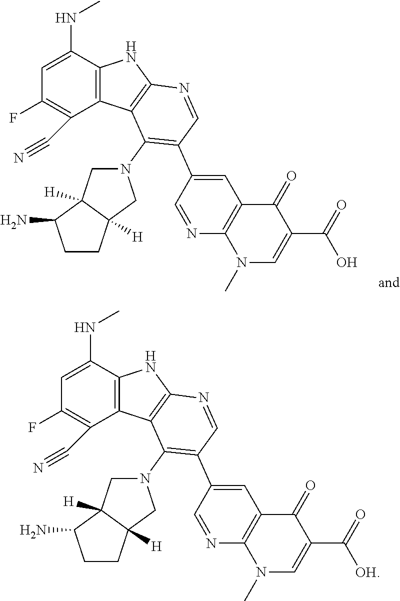 [SMILES: CNc1cc(F)c(C#N)c2c1[nH]c1ncc(-c3cnc4c(c3)c(=O)c(C(=O)O)cn4C)c(N3C[C@H]4CC[C@@H](N)[C@H]4C3)c12 ; CNc1cc(F)c(C#N)c2c1[nH]c1ncc(-c3cnc4c(c3)c(=O)c(C(=O)O)cn4C)c(N3C[C@@H]4CC[C@H](N)[C@@H]4C3)c12]

The way of showing relative stereochemistry also applies to the final compounds.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgment of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

Inhibitors of DNA Gyrase and/or Topoisomerase IV

The present invention relates to a compound of formula (I),

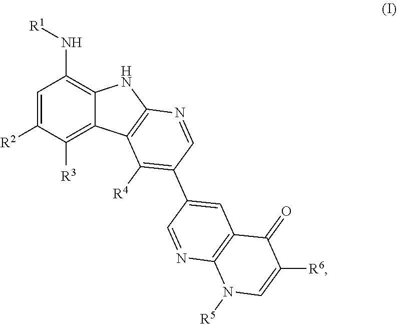

wherein

R¹ is C₁₋₆alkyl;

R² is halogen;

R³ is halogen or cyano;

R⁴ is 1,3,4,5,6,6a-hexahydrocyclopenta[c]pyrrolyl substituted by amino;
2,3,3a,4,6,6a-hexahydropyrrolo[2,3-c]pyrrolyl substituted by amino;
3,3a,4,5,6,6a-hexahydro-1H-cyclopenta[c]pyrrolyl substituted by amino;
azabicyclo[3.1.0]hexanyl substituted by amino or aminoC₁₋₆alkyl;
azaspiro[2.4]heptanyl substituted by amino;
azaspiro[2.5]octanyl substituted by amino;
azaspiro[3.3]heptanyl substituted by amino;
piperidinyl substituted twice by aminoC₁₋₆alkyl and halogen;
pyrrolidinyl substituted once or twice by substituents independently selected from amino, aminoC₁₋₆alkyl, aminoC₃₋₇cycloalkyl, haloC₁₋₆alkyl and halogen;
R⁵ is C₁₋₆alkyl;
R⁶ is carboxy;
or a pharmaceutically acceptable salt thereof.

A further embodiment of present invention is (ii) a compound of formula (I) according to (i), wherein
R¹ is methyl;
R² is fluoro;
R³ is chloro, fluoro or cyano;
R⁴ is aminocyclopropylpyrrolidinyl; amino(trifluoromethyl)pyrrolidinyl; amino-1,3,4,5,6,6a-hexahydrocyclopenta[c]pyrrolyl; amino-3,3 a,4,5,6,6a-hexahydro-1H-cyclopenta[c]pyrrolyl; aminoazabicyclo[3.1.0]hexanyl; aminoazaspiro[2.4]heptanyl; aminoazaspiro[2.5]octanyl; aminoazaspiro[3.3]heptanyl; aminomethyl(fluoro)piperidinyl; aminomethyl(fluoro)pyrrolidinyl; aminomethylazabicyclo[3.1.0]hexanyl; aminomethylpyrrolidinyl; aminopropyl-2,3,3a,4,6,6a-hexahydropyrrolo[2,3-c]pyrrolyl; or aminopyrrolidinyl;
R⁵ is methyl or ethyl;
R⁶ is carboxy;

or a pharmaceutically acceptable salt thereof.

A further embodiment of present invention is (iii) a compound of formula (I) according to (i) or (ii), or a pharmaceutically acceptable salt thereof, wherein R⁴ is azaspiro[2.4]heptanyl substituted by amino; pyrrolidinyl substituted once or twice by substituents independently selected from amino, aminoC₁₋₆alkyl, aminoC₃₋₇cycloalkyl, haloC₁₋₆alkyl and halogen.

A further embodiment of present invention is (iv) a compound of formula (I) according to any one of (i) to (iii), or a pharmaceutically acceptable salt thereof, wherein R⁴ is aminoazaspiro[2.4]heptanyl; aminocyclopropylpyrrolidinyl; amino(trifluoromethyl)pyrrolidinyl; aminomethyl(fluoro)pyrrolidinyl; aminomethylpyrrolidinyl; or aminopyrrolidinyl.

A further embodiment of present invention is (v) a compound of formula (I) according to any one of (i) to (iv), or a pharmaceutically acceptable salt thereof, wherein R⁵ is methyl.

A further embodiment of present invention is (vi) a compound of formula (I) according to any one of (i) to (v), wherein
R¹ is C₁₋₆alkyl;
R² is halogen;
R³ is halogen or cyano;
R⁴ is azaspiro[2.4]heptanyl substituted by amino; or
   pyrrolidinyl substituted once or twice by substituents independently selected from amino, aminoC₁₋₆alkyl, aminoC₃₋₇cycloalkyl, haloC₁₋₆alkyl and halogen;
R⁵ is C₁₋₆alkyl;
R⁶ is carboxy;
or a pharmaceutically acceptable salt thereof.

A further embodiment of present invention is (vii) a compound of formula (I) according to any one of (i) to (vi),wherein
R¹ is methyl;
R² is chloro or fluoro;
R³ is chloro, fluoro or cyano;
R⁴ is aminoazaspiro[2.4]heptanyl; aminocyclopropylpyrrolidinyl; amino(trifluoromethyl)pyrrolidinyl; aminomethyl(fluoro)pyrrolidinyl; aminomethylpyrrolidinyl; or aminopyrrolidinyl;
R⁵ is methyl;
R⁶ is carboxy;

or a pharmaceutically acceptable salt thereof.

Another embodiment of present invention is that compounds of formula (I) are selected from:

6-[4-[3-(1-Aminocyclopropyl)pyrrolidin-1-yl]-5,6-difluoro-8-(methylamino)-9H-pyrido[2,3-b]indol-3-yl]-1-methyl-4-oxo-1,8-naphthyridine-3-carboxylic acid;

6-[4-(7-Amino-2-azaspiro[3.3]heptan-2-yl)-5,6-difluoro-8-(methylamino)-9H-pyrido[2,3-b]indol-3-yl]-1-methyl-4-oxo-1,8-naphthyridine-3-carboxylic acid;

6-[4-(3-aminopyrrolidin-1-yl)-5,6-difluoro-8-(methylamino)-9H-pyrido[2,3-b]indol-3-yl]-1-ethyl-4-oxo-1,8-naphthyridine-3-carboxylic acid;

6-(4-(3-(Aminomethyl)pyrrolidin-1-yl)-5,6-difluoro-8-(methylamino)-9H-pyrido[2,3-b]indol-3-yl)-1-ethyl-4-oxo-1,4-dihydro-1,8-naphthyridine-3-carboxylic acid;

6-(4-(3-Amino-3-(trifluoromethyl)pyrrolidin-1-yl)-5,6-difluoro-8-(methylamino)-9H-pyrido[2,3-b]indol-3-yl)-1-methyl-4-oxo-1,4-dihydro-1,8-naphthyridine-3-carboxylic acid;

6-[4-[1-(aminomethyl)-3-azabicyclo[3.1.0]hexan-3-yl]-5,6-difluoro-8-(methylamino)-9H-pyrido[2,3-b]indol-3-yl]-1-methyl-4-oxo-1,8-naphthyridine-3-carboxylic acid;

cis-6-[5,6-difluoro-8-(methylamino)-4-[5-(3-aminopropyl)-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;

6-[5,6-difluoro-8-(methylamino)-4-[rel-(1S,5R)-6-amino-3-azabicyclo[3.1.0]hexan-3-yl]-9H-pyrido[2,3-b]indol-3-yl]-1-methyl-4-oxo-1,8-naphthyridine-3-carboxylic acid;

6-[4-[2-(Aminomethyl)pyrrolidin-1-yl]-5,6-difluoro-8-(methylamino)-9H-pyrido[2,3-b]indol-3-yl]-1-methyl-4-oxo-1,8-naphthyridine-3-carboxylic acid;

6-[4-[(2S)-2-(Aminomethyl)pyrrolidin-1-yl]-5,6-difluoro-8-(methylamino)-9H-pyrido[2,3-b]indol-3-yl]-1-methyl-4-oxo-1,8-naphthyridine-3-carboxylic acid;

6-[4-[3-(Aminomethyl)pyrrolidin-1-yl]-5,6-difluoro-8-(methylamino)-9H-pyrido[2,3-b]indol-3-yl]-1-methyl-4-oxo-1,8-naphthyridine-3-carboxylic acid;

trans-6-(5,6-Difluoro-8-(methylamino)-4-[3a-amino-1,3,4,5,6,6a-hexahydrocyclopenta[c]pyrrol-2-yl]-9H-pyrido[2,3-b]indol-3-yl)-1-methyl-4-oxo-1,8-naphthyridine-3-carboxylic acid;

6-[5-Cyano-6-fluoro-8-(methylamino)-4-[rel-(3aR,4R,6aS)-4-amino-3,3a,4,5,6,6a-hexahydro-1H-cyclopenta[c]pyrrol-2-yl]-9H-pyrido[2,3-b]indol-3-yl]-1-methyl-4-oxo-1,8-naphthyridine-3-carboxylic acid;

6-[4-[3-(Aminomethyl)-3-fluoro-pyrrolidin-1-yl]-5,6-difluoro-8-(methylamino)-9H-pyrido[2,3-b]indol-3-yl]-1-ethyl-4-oxo-1,8-naphthyridine-3-carboxylic acid;

6-[4-[3-(Aminomethyl)-3-fluoro-1-piperidyl]-5,6-difluoro-8-(methylamino)-9H-pyrido[2,3-b]indol-3-yl]-1-ethyl-4-oxo-1,8-naphthyridine-3-carboxylic acid;

6-[4-[3-(Aminomethyl)-3-fluoro-pyrrolidin-1-yl]-5,6-difluoro-8-(methylamino)-9H-pyrido[2,3-b]indol-3-yl]-1-methyl-4-oxo-1,8-naphthyridine-3-carboxylic acid;

6-[4-[(3R)-3-Aminopyrrolidin-1-yl]-5,6-difluoro-8-(methylamino)-9H-pyrido[2,3-b]indol-3-yl]-1-methyl-4-oxo-1,8-naphthyridine-3-carboxylic acid;

trans-6-[4-(2-amino-5-azaspiro[2.4]heptan-5-yl)-5,6-difluoro-8-(methylamino)-9H-pyrido[2,3-b]indol-3-yl]-1-methyl-4-oxo-1,8-naphthyridine-3-carboxylic acid;

cis-6-[4-(2-amino-5-azaspiro[2.4]heptan-5-yl)-5,6-difluoro-8-(methylamino)-9H-pyrido[2,3-b]indol-3-yl]-1-methyl-4-oxo-1,8-naphthyridine-3-carboxylic acid;

6-[4-[(2R,3R)-2-amino-5-azaspiro[2.4]heptan-5-yl]-5,6-difluoro-8-(methylamino)-9H-pyrido[2,3-b]indol-3-yl]-1-methyl-4-oxo-1,8-naphthyridine-3-carboxylic acid;

6-[4-[(2S,3R)-2-amino-5-azaspiro[2.4]heptan-5-yl]-5,6-difluoro-8-(methylamino)-9H-pyrido[2,3-b]indol-3-yl]-1-methyl-4-oxo-1,8-naphthyridine-3-carboxylic acid;

6-[4-[(2R,3S)-2-amino-5-azaspiro[2.4]heptan-5-yl]-5,6-difluoro-8-(methylamino)-9H-pyrido[2,3-b]indol-3-yl]-1-methyl-4-oxo-1,8-naphthyridine-3-carboxylic acid;

6-[4-[(2S,3S)-2-amino-5-azaspiro[2.4]heptan-5-yl]-5,6-difluoro-8-(methylamino)-9H-pyrido[2,3-b]indol-3-yl]-1-methyl-4-oxo-1,8-naphthyridine-3-carboxylic acid;

6-[4-[(2R,3R)-2-amino-5-azaspiro[2.4]heptan-5-yl]-5-chloro-6-fluoro-8-(methylamino)-9H-pyrido[2,3-b]indol-3-yl]-1-methyl-4-oxo-1,8-naphthyridine-3-carboxylic acid;

6-[4-[(2S,3S)-2-amino-5-azaspiro[2.4]heptan-5-yl]-5-chloro-6-fluoro-8-(methylamino)-9H-pyrido[2,3-b]indol-3-yl]-1-methyl-4-oxo-1,8-naphthyridine-3-carboxylic acid;

6-[4-[(2S,3R)-2-amino-5-azaspiro[2.4]heptan-5-yl]-5-chloro-6-fluoro-8-(methylamino)-9H-pyrido[2,3-b]indol-3-yl]-1-methyl-4-oxo-1,8-naphthyridine-3-carboxylic acid;

6-[4-[(2R,3S)-2-amino-5-azaspiro[2.4]heptan-5-yl]-5-chloro-6-fluoro-8-(methylamino)-9H-pyrido[2,3-b]indol-3-yl]-1-methyl-4-oxo-1,8-naphthyridine-3-carboxylic acid;

6-[4-[(2S,3R)-2-amino-5-azaspiro[2.4]heptan-5-yl]-5-cyano-6-fluoro-8-(methylamino)-9H-pyrido[2,3-b]indol-3-yl]-1-methyl-4-oxo-1,8-naphthyridine-3-carboxylic acid;

6-[4-[(2R,3S)-2-amino-5-azaspiro[2.4]heptan-5-yl]-5-cyano-6-fluoro-8-(methylamino)-9H-pyrido[2,3-b]indol-3-yl]-1-methyl-4-oxo-1,8-naphthyridine-3-carboxylic acid;

6-[4-[(2S,3S)-2-amino-5-azaspiro[2.4]heptan-5-yl]-5-cyano-6-fluoro-8-(methylamino)-9H-pyrido[2,3-b]indol-3-yl]-1-methyl-4-oxo-1,8-naphthyridine-3-carboxylic acid;

6-[4-[(2R,3R)-2-amino-5-azaspiro[2.4]heptan-5-yl]-5-cyano-6-fluoro-8-(methylamino)-9H-pyrido[2,3-b]indol-3-yl]-1-methyl-4-oxo-1,8-naphthyridine-3-carboxylic acid;

6-[4-[(2R,3R)-2-amino-5-azaspiro[2.5]octan-5-yl]-5,6-difluoro-8-(methylamino)-9H-pyrido[2,3-b]indol-3-yl]-1-methyl-4-oxo-1,8-naphthyridine-3-carboxylic acid; and

6-[4-[(2S,3S)-2-amino-5-azaspiro[2.5]octan-5-yl]-5,6-difluoro-8-(methylamino)-9H-pyrido[2,3-b]indol-3-yl]-1-methyl-4-oxo-1,8-naphthyridine-3-carboxylic acid;

or a pharmaceutically acceptable salt thereof.

### Synthesis

The compounds of the present invention can be prepared by any conventional means. Suitable processes for synthesizing these compounds as well as their starting materials are provided in the schemes below and in the examples. All substituents, in particular, R¹ to R⁶ are as defined above unless otherwise indicated. Furthermore, and unless explicitly otherwise stated, all reactions, reaction conditions, abbreviations and symbols have the meanings well known to a person of ordinary skill in organic chemistry.

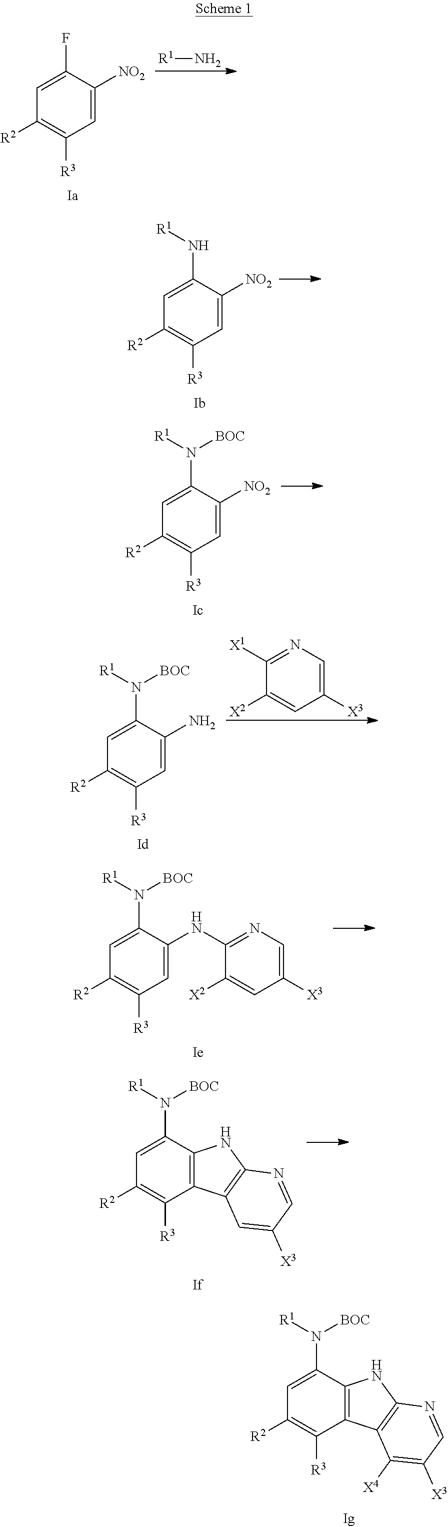

wherein X¹, X², X³ and X⁴ are halogen.

Compound of formula (Ig) can be prepared according to Scheme 1. Nucleophilic substitution of ortho-fluoro nitrobenzene (Ia) with amine R¹-NH₂ affords aniline (Ib). The aniline (Ib) can be protected with di-tert-butyl carbonate to give the protected aniline (Ic). The nitro group in aniline (Ic) can be reduced by a reducing agent, such as H₂ with palladium catalysts, to give the compound of formula (Id). Coupling of the compound of formula (Id) with tri-halogenated pyridine can be achieved using palladium catalysts and phosphine ligands to give compound of formula (Ie). Cyclization of compound of formula (Ie) using palladium catalysts and phosphine ligands gives compound of formula (If). Compound of formula (Ig) can be obtained through oxidation of the pyridine of compound of formula (If) followed by halogenation, such as treatment of POCl₃ or POBr₃. Compound of formula (Ig) can also be obtained by additional halogenation of compound of formula (Ig) (when R³ is H) with halogenating reagent, such as NCS, followed by di-tert-butyl carbonate re-protection.

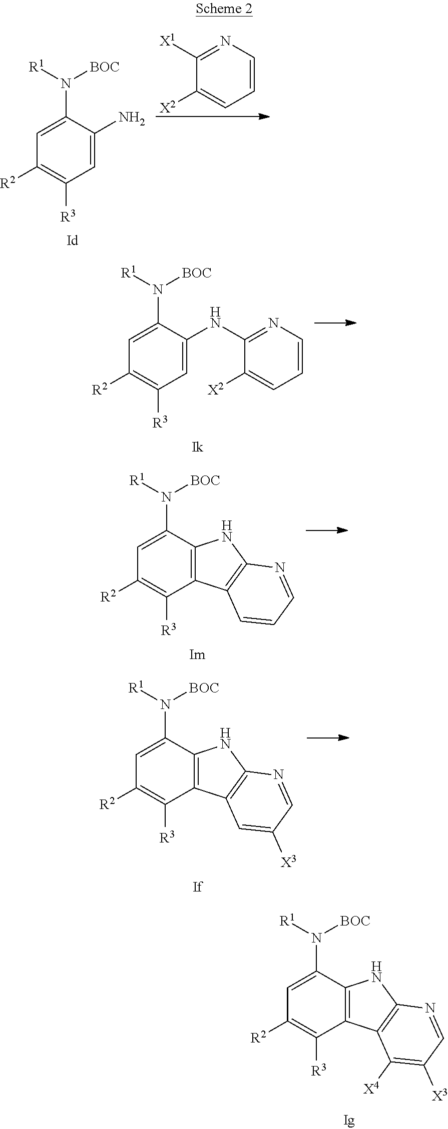

wherein X¹, X², X³ and X⁴ are halogen.

Alternatively, compound of formula (Ig) can be prepared according to Scheme 2. Coupling of the compound of formula (Id) with di-halogenated pyridine can be achieved using palladium catalyst and phosphine ligands to give compound of formula (Ik). Cyclization of compound of formula (Ik) using palladium catalyst and phosphine ligands gives compound of formula (Im). Compound of formula (Im) can be subject to halogenation using halogenating reagent, such as NCS, NBS or NIS, to give compound of formula (If), which subsequently undergoes oxidation of the pyridine followed by halogenation, such as treatment of POCl₃ or POBr₃. Alternatively, compound of formula (If) can be obtained by treatment of compound of formula (If) (when R³ is H) with halogenating reagent, such as NBS, to give compound of formula (If) (when R³ is halogen, in particular bromo). Compound of formula (If) can also be obtained by converting compound of formula (If) (when R³ is halogen, in particular bromo) to compound of formula (If) (when R³ is CN) via palladium mediated substitution or nucleophilic substitution. Compound of formula (Ig) can then be obtained through oxidation of the pyridine of compound of formula (If) followed by halogenation, such as treatment of POCl₃ or POBr₃.

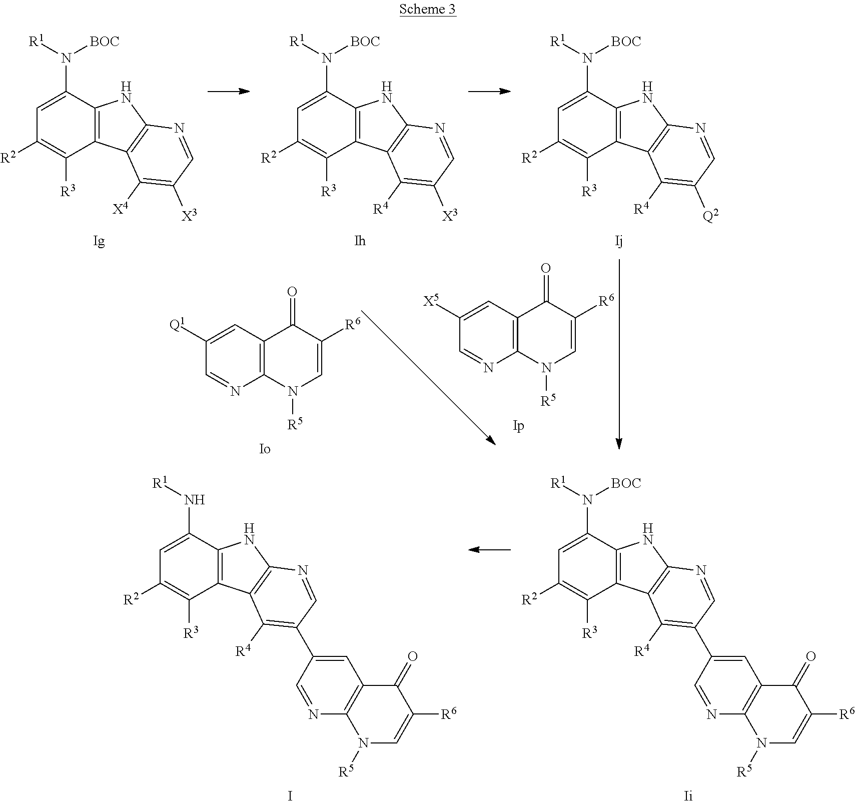

wherein X³ and X⁴ are halogen, X⁵ are halogen or OTf; Q¹ and Q² are boronic acids or esters.

Compound of formula (I) can be prepared according to Scheme 3. Introducing R⁴ to compound of formula (Ig) can be achieved either through nucleophilic substitution with amine and a base for certain C—N bond formation (with R⁴ bearing a nucleophilic N), or a Buchwald-Hartwig Cross Coupling Reaction for certain C—N bond formation (with R⁴ bearing a basic N), to give compound of formula (Ih). Further coupling of compound of formula (Ih) with compound of formula (Io) to give compound of formula (Ii) can be achieved using a palladium catalyzed Suzuki coupling. Chiral separation can be achieved on compound of formula (Ih) or compound of formula (Ii). Some special compound of formula (Ii) need to reverse the Suzuki coupling for C—C bond formation by converting compound of formula (Ih) to compound of formula (Ij) as a boronic ester or boronic acid then coupling with compound of formula (Ip). Ester hydrolysis such as NaOH in ethanol followed by deprotection of compound of formula (Ii) in the presence of an acid, such as trifluoroacetic acid, then affords compound of formula (I).

This invention also relates to a process for the preparation of a compound of formula (I) comprising the reaction of compound of formula (Ii),

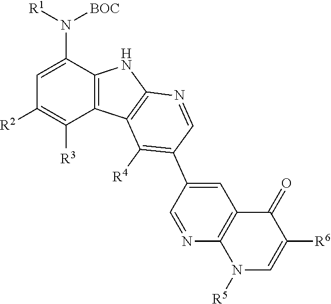

with an acid, which can be for example trifluoroacetic acid;

wherein R¹ to R⁶ are defined above. A compound of formula (I) when manufactured according to the above process is also an object of the invention.

### Pharmaceutical Compositions and Administration

Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to reduced bacterial load or improve host survival through the inhibition of bacterial DNA gyrase and/or Topoisomerase IV. For example, such amount may be below the amount that is toxic to normal cells, or the mammal as a whole.

In one example, the pharmaceutically effective amount of the compound of the invention administered parenterally per dose will be in the range of about 0.1 to 1000 mg/kg, alternatively about 1 to 100 mg/kg of patient body weight per day. In another embodiment, oral unit dosage forms, such as tablets and capsules, preferably contain from about 5 to about 5000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 10 to 500 mg of the compound of the invention compounded with about 40 to 400 mg anhydrous lactose, about 5 to 50 mg sodium croscarmellose, about 5 to 50 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 1000 mg) of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I), or a stereoisomer or pharmaceutically acceptable salt thereof. In a further embodiment includes a pharmaceutical composition comprising a compound of formula (I), or a stereoisomer or pharmaceutically acceptable salt thereof, together with a pharmaceutically acceptable carrier or excipient.

Another embodiment includes a pharmaceutical composition comprising a compound of formula (I) for use in the treatment and/or prophylaxis of bacterial infections.

In some embodiments, the compounds of this invention may be administered, as part of a single or multiple dosage regimen, orally, parenterally, by inhalation spray, topically, rectally, nasally, buccally, vaginally, or via an implanted reservoir. The term parenteral as used includes subcutaneous, intracutaneous, intravenous, intramuscular, intra-articular, intrasynovial, intrasternal, intrathecal, intralesional and intracranial injection or infusion techniques. Typically, the pharmaceutical compositions of the invention will be administered from about 1 to 5 times per day or alternatively, as a continuous infusion upon improvement of a patient's condition.

### Indications and Methods of Treatment

The compounds of the invention are useful for treatment and/or prophylaxis of bacterial infection in humans or other animals by administering to the subject in need of a therapeutically effective amount of compound of formula (I), or a pharmaceutically acceptable salt, or enantiomer or diastereomer thereof. The compounds and methods of the invention are particularly well suited for human patients infected by pathogens that include Staphylococcus aureus, Escherichia coli, Klebsiella pneumoniae, Acinetobacter baumannii and Pseudomonas aeruginosa. Examples of bacterial organisms that may also be controlled by the compounds of the invention include, but not limited to, the following Gram-Positive and Gram-Negative organisms: Streptococcus pneumoniae, Streptococcus pyogenes, Enterococcus faecalis, Enterococcus faecium, Enterobacter spp. species, Proteus spp. species, Serratia marcescens, Staphylococcus aureus, Coag. Neg. Staphylococci, Haemophilus influenzae, Bacillus anthraces, Mycoplasma pneumoniae, Moraxella catarrhalis, Chlamydophila pneumoniae, Chlamydia trachomatis, Legionella pneumophila, Mycobacterium tuberculosis, Helicobacter pylori, Staphylococcus saprophyticus, Staphylococcus epidermidis, Francisella tularensis, Yersinia pestis, Clostridium difficile, Bacteroides spp. species Neisseria gonorrhoeae, Neisseria meningitidis, Burkholderia pseudomallei, Burkholderia mallei, Borrelia burgdorferi, Mycobacterium avium complex, Mycobacterium abscessus, Mycobacterium kansasii, E. coli and Mycobacterium ulcerans.

Examples of bacterial infections may include, but not limited to, upper respiratory infections, lower respiratory infections, ear infections, pleuropulmonary and bronchial infections, complicated urinary tract infections, uncomplicated urinary tract infections, intra-abdominal infections, cardiovascular infections, a blood stream infection, sepsis, bacteremia, CNS infections, skin and soft tissue infections, GI infections, bone and joint infections, genital infections, eye infections, or granulomatous infections. Examples of specific bacterial infections include, but not limited to, uncomplicated skin and skin structure infections (uSSSI), complicated skin and skin structure infections (cSSSI), catheter infections, pharyngitis, sinusitis, otitis extema, otitis media, bronchitis, empyema, pneumonia, community-acquired bacterial pneumoniae (CABP), hospital-acquired pneumonia (HAP), hospital-acquired bacterial pneumonia, ventilator-associated pneumonia (VAP), diabetic foot infections, vancomycin resistant enterococci infections, cystitis and pyelonephritis, renal calculi, prostatitis, peritonitis, complicated intra-abdominal infections (cIAI) and other inter-abdominal infections, dialysis-associated peritonitis, visceral abscesses, endocarditis, myocarditis, pericarditis, transfusion-associated sepsis, meningitis, encephalitis, brain abscess, osteomyelitis, arthritis, genital ulcers, urethritis, vaginitis, cervicitis, gingivitis, conjunctivitis, keratitis, endophthalmitisa, an infection in cystic fibrosis patients or an infection of febrile neutropenic patients.

Furthermore, the invention relates to the use of a compound of formula (I) for the treatment and/or prophylaxis of bacterial infection. The invention relates to the use of a compound of formula (I) for the preparation of a medicament for the treatment and/or prophylaxis of bacterial infection. Another embodiment includes a method for the treatment or prophylaxis of bacterial infection which method comprises administering an effective amount of a compound of formula (I), or pharmaceutically acceptable salt, or enantiomer or diastereomer thereof.

### EXAMPLES

The invention will be more fully understood by reference to the following examples. They should not, however, be construed as limiting the scope of the invention.

### Abbreviations

Abbreviations used herein are as follows:
ACN or MeCN acetonitrile
B₂Pin₂ Bis(pinacolato)diboron
BINAP 2,2′-Bis(diphenylphosphino)-1,1′-binaphthalene
cataCXium A-Pd-G2 Chloro[(di(1-adamantyl)-N-butylphosphine)-2-(2-aminobiphenyl)]palladium(II)
CC₅₀ concentration results in the death of 50 percent of the cells
DBU 1,8-Diazabicyclo[5.4.0]undec-7-ene
DCM dichloromethane
DIPEA N,N-diisopropylethylamine
DPPA diphenylphosphoryl azide
EtOAc or EA ethyl acetate
FA formic acid
h(s) or hr(s) hour
HPLC: high performance liquid chromatography
HPLC-UV: high performance liquid chromatography with ultraviolet detector
IV intravenous
m-CPBA meta-chloroperoxybenzoic acid
MIC minimum inhibitory concentration
OD optical density

Pd-Ad₂nBuP Biphenyl Chloro[(di(1-adamantyl)-N-butylphosphine)-2-(2-aminobiphenyl)]palladium(II)
PE petroleum ether
Precat precatalyst
prep-HPLC preparative high performance liquid chromatography
rel relative
rt room temperature
Rt retention time
SFC supercritical fluid chromatography
TLC Thin Layer Chromatography
UV ultraviolet detector
wt weight
x-phos Pd G2 Chloro(2-dicyclohexylphosphino-2′,4′,6′-triisopropyl-1,1′-biphenyl)[2-(2′-amino-1,1′-biphenyl)]palladium(II)

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using X Bridge™ Perp C₁₈ (5 μm, OBD™ 30×100 mm) column or SunFire™ Perp C₁₈ (5 μm, OBD™ 30×100 mm) column.

Chiral Separation was conducted on Thar 350 preparative SFC using ChiralPak AD-10μ (200×50 mm I.D.) with mobile phase A for CO₂ and B for ethanol. LC/MS spectra were obtained using a Waters UPLC-SQD Mass. Standard LC/MS conditions were as follows (running time: 3 minutes):

Acidic condition: A: 0.1% formic acid and 1% acetonitrile in H₂O; B: 0.1% formic acid in acetonitrile;

Basic condition: A: 0.05% NH₃.H₂O in H₂O; B: acetonitrile.

Mass spectra (MS): generally only ions which indicate the parent mass are reported, and unless otherwise stated the mass ion quoted is the positive mass ion (M+H)⁺.

NMR Spectra were obtained using Bruker Avance 400 MHz.

All reactions involving air-sensitive reagents were performed under an nitrogen/or argon atmosphere. Reagents were used as received from commercial suppliers without further purification unless otherwise noted.

### PREPARATIVE EXAMPLES

### Intermediate A1

### tert-Butyl N-(3,4-dichloro-6-fluoro-9H-pyrido[2,3-b]indol-8-yl)-N-methyl-carbamate

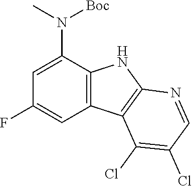 [SMILES: CN(C(=O)OC(C)(C)C)c1cc(F)cc2c1[nH]c1ncc(Cl)c(Cl)c12]

The titled compound was synthesized according to the following scheme:

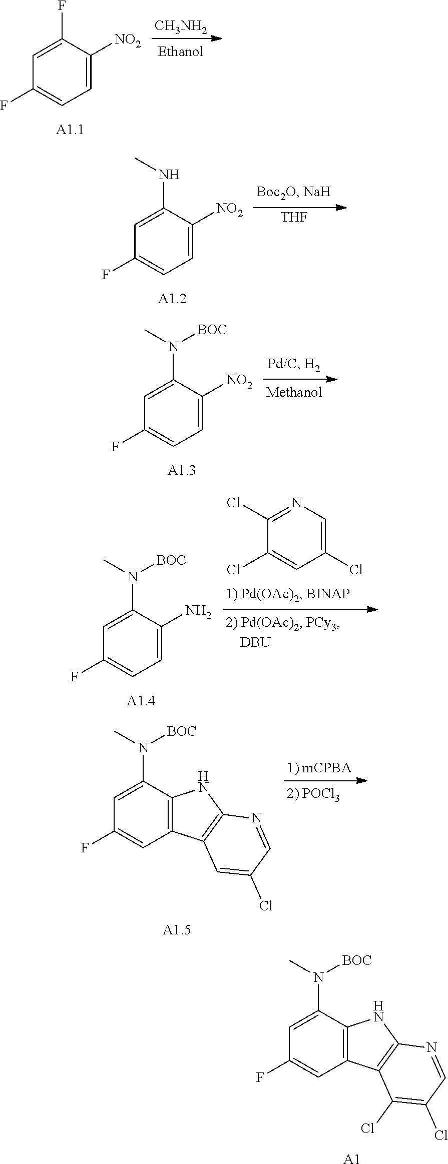

### Step (a) Preparation of 5-fluoro-N-methyl-2-nitro-aniline (compound A1.2)

Methylamine solution (355 g, 2.866 mol, 25% in EtOH) was added dropwise to 2,4-difluoronitrobenzene (147 g, 0.924 mol) at 0° C. over 15 min. After completion of the addition, the reaction mixture was stirred at 0° C. for 2 h. The solution was diluted with ethanol (500 mL) and poured into 2 L of ice-water. The resulting precipitates were collected by filtration and dried in vacuo to give 5-fluoro-N-methyl-2-nitro-aniline (143 g, 63% yield) as a yellow solid.

### Step (b) Preparation of tert-butyl N-(5-fluoro-2-nitro-phenyl)-N-methyl-carbamate (compound A1.3)

To a suspension of sodium hydride (141 g, 3.5 mol, 60% dispersion in mineral oil) in dry TRF (2 L) was added 5-fluoro-N-methyl-2-nitro-aniline (60 g, 0.35 mol, compound A1.2) portion wise at 0° C. After the solution was stirred at 0° C. for 1 h, a solution of di-tert-butyl dicarbonate (115 g, 0.53 mol) in TRF (0.5 L) was added dropwise and the reaction mixture continued stirring for another 15 h at 15° C. The reaction mixture was poured into 1.6 L of ice-water and extracted with EtOAc (1.6 L) two times. The combined organics were dried over anhy. sodium sulfate and concentrated in vacuo. The crude product was purified by silica gel flash chromatography (1-5% EtOAc in petroleum ether) to give tert-butyl N-(5-fluoro-2-nitro-phenyl)-N-methyl-carbamate (70 g, 73% yield) as a yellow solid. MS (ESI): 293.0 ([M+Na]⁺), 171.0 ([M-C₄H₈—CO₂+H]⁺).

### Step (c) Preparation of tert-butyl N-(2-amino-5-fluoro-phenyl)-N-methyl-carbamate (compound A1.4)

To a solution of tert-butyl N-(5-fluoro-2-nitro-phenyl)-N-methyl-carbamate (70 g, 259 mmol, compound A1.3) in MeOH (1 L) was added palladium on carbon (5 g, 10 wt. % loading). The reaction mixture was stirred at 16° C. for 18 h under H₂ atmosphere (50 psi). After the remaining palladium catalyst was removed by filtration, the filtrate was concentrated in vacuo to give tert-butyl N-(2-amino-5-fluoro-phenyl)-N-methyl-carbamate (60 g, 96% yield) as a white solid. MS (ESI): 263.1 ([M+Na]⁺), 185.0 ([M-C₄H_{8 +}H]⁺), 141.0 ([M-C₄H₈—CO2+H]⁺).

### Step (d) Preparation of tert-butyl N-(3-chloro-6-fluoro-9H-pyrido[2,3-b]indol-8-yl)-N-methyl-carbamate (compound A1.5)

To a solution of tert-butyl N-(2-amino-5-fluoro-phenyl)-N-methyl-carbamate (80 g, 333 mmol, compound A1.4) and 2,3,5-trichloropyridine (66.8 g, 366 mmol, CAS: 16063-70-0) in dioxane (2 L) were added cesium carbonate (217 g, 666 mmol), palladium(II) acetate (3.74 g, 16.7 mmol), and BINAP (20.7 g, 33.3 mmol, CAS: 98327-87-8). The reaction mixture was stirred at 120° C. for 16 h under nitrogen atmosphere. After the reaction mixture was cooled back to room temperature, it was diluted with EtOAc (800 mL). The precipitate was removed by filtration and the filtrate was concentrated in vacuo, and the crude product was purified by silica gel flash chromatography (0.2% to 5% EtOAc in petroleum ether) to give intermediate tert-butyl N-[2-[(3,5-dichloro-2-pyridyl)amino]-5-fluoro-phenyl]-N-methyl-carbamate (75 g, 58% yield) as a white solid. MS (ESI): 390.1 ([{³⁷Cl}M+H]⁺), 388.1 ([{³⁷Cl+³⁵Cl}M+H]⁺), 386.1 ([{³⁵Cl}M+H]⁺).

Next, to a solution of tert-butyl N-[2-[(3,5-dichloro-2-pyridyl)amino]-5-fluoro-phenyl]-N-methyl-carbamate (5 g, 12.95 mmol) and DBU (3.94 g, 25.9 mmol, CAS: 6674-22-2) in the mixture of o-xylene (7.5 mL) and N,N-dimethylacetamide (7.5 mL) were added palladium(II) acetate (727 mg, 3.24 mmol) and tricyclohexylphosphine tetrafluoroborate (2.38 g, 6.48 mmol) under nitrogen atmosphere. The reaction mixture was stirred at 160° C. for 6 h before it was cooled back to room temperature and poured into water (100 mL). The mixture was extracted with EtOAc (200 mL) and the organic layer was collected and washed with water (50 mL) two times, brine (30 mL) two times, and dried over anhy. sodium sulfate. The separated organic layer was concentrated in vacuo to give a crude product, which was purified by silica gel flash chromatography (0.5% to 20% EtOAc in DCM) to give tert-butyl N-(3-chloro-6-fluoro-9H-pyrido[2,3-b]indol-8-yl)-N-methyl-carbamate (873 mg, 19.3% yield) as a yellow solid. MS (ESI): 352.1 ([{³⁷Cl}M+H]⁺), 350.1 ([{³⁵Cl}M+H]⁺).

### Step (e) Preparation of tert-butyl N-(3,4-dichloro-6-fluoro-9H-pyrido[2,3-b]indol-8-yl)-N-methyl-carbamate (Intermediate A1)

To a solution of tert-butyl N-(3-chloro-6-fluoro-9H-pyrido[2,3-b]indol-8-yl)-N-methyl-carbamate (4.8 g, 13.7 mmol, compound A1.5) in DCM (200 mL) was added 3-chloroperbenzoic acid (9.47 g, 54.9 mmol) at 0° C. under nitrogen atmosphere. Afterwards, the reaction mixture was allowed to warm up to 30° C. and stirred for 12 h. Then the reaction mixture was cooled back to room temperature and poured into aq. sodium sulfite solution (10%, 150 mL) and stirred for 1 h followed by extraction with EtOAc (750 mL) three times. The combined organics were washed by aq. sodium bicarbonate solution (5N, 200 mL), brine (250 mL), dried over anhy. sodium sulfate, and concentrated in vacuo to give a crude product of tert-butyl N-(3-chloro-6-fluoro-1-oxido-9H-pyrido [2,3-b]indol-1-ium-8-yl)-N-methyl-carbamate (4.8 g, 96% yield) as a brown solid. MS (ESI): 368.1 ([{³⁷Cl}M+H]⁺), 366.1 ([{³⁵Cl}M+H]⁺).

Next, to a solution of tert-butyl N-(3-chloro-6-fluoro-1-oxido-9H-pyrido[2,3-b] indol-1-ium-8-yl)-N-methyl-carbamate (4.8 g, 13.1 mmol) in DMF (100 mL) was added phosphorus(V) oxychloride (22.1 g, 144 mmol) dropwise at −5° C. The mixture was stirred at −5° C. to 0° C. for 1 h before poured into satd. aq. sodium bicarbonate solution (350 mL) at 0° C. The mixture was then extracted by EtOAc (500 mL) three times and the combined organics were washed with water (200 mL) three times, brine (150 mL) two times, dried over anhy. sodium sulfate, and concentrated in vacuo. The crude product was then purified by washing with MeOH (120 mL) to give tert-butyl N-(3,4-dichloro-6-fluoro-9H-pyrido[2,3-b]indol-8-yl)-N-methyl-carbamate (2.16 g, 42.9% yield) as a pale yellow solid. MS (ESI): 388.1 ([{³⁷Cl+³⁷Cl}M+H]⁺), 386.1 ([{³⁷Cl+³⁵Cl}M+H]⁺), 384.1 ([{³⁵Cl+³⁵Cl}M+H]⁺). ¹H NMR (400 MHz, DMSO-d6) δppm: 12.55 (s, 1H), 8.65 (s, 1H), 8.05 (d, J=7.0 Hz, 1H) 7.49 (dd, J=10.3, 2.5 Hz, 1H) 3.26 (s, 3H) 1.19-1.62 (m, 9H).

### Intermediate A2

### tert-Butyl N-(3,4-dichloro-5,6-difluoro-9H-pyrido[2,3-b]indol-8-yl)-N-methyl-carbamate

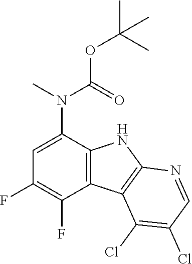 [SMILES: CN(C(=O)OC(C)(C)C)c1cc(F)c(F)c2c1[nH]c1ncc(Cl)c(Cl)c12]

In analogy to the synthesis of tert-Butyl N-(3,4-dichloro-6-fluoro-9H-pyrido[2,3-b]indol-8-yl)-N-methyl-carbamate (Intermediate A1), the title compound was prepared by replacing 2,4-difluoronitrobenzene with 2,4,5-trifluoronitrobenzene in step (a). MS (ESI): 406.1 ([{³⁷Cl+³⁷Cl}M+H]⁺), 404.1 ([{³⁷Cl+³⁵Cl}M+H]⁺), 402.1 ([{³⁵Cl+³⁵Cl}M+H]⁺). ¹H NMR (400 MHz, DMSO-d6) δ ppm: 12.84 (br. s, 1H), 8.64 (s, 1H), 7.70 (m, 1H), 3.22 (s, 3H), 1.22-1.50 (m, 9H).

### Intermediate A3

### tert-Butyl methyl(3,4,5-trichloro-6-fluoro-9H-pyrido[2,3-b]indol-8-yl)carbamate

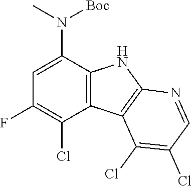 [SMILES: CN(C(=O)OC(C)(C)C)c1cc(F)c(Cl)c2c1[nH]c1ncc(Cl)c(Cl)c12]

The titled compound was synthesized according to the following scheme:

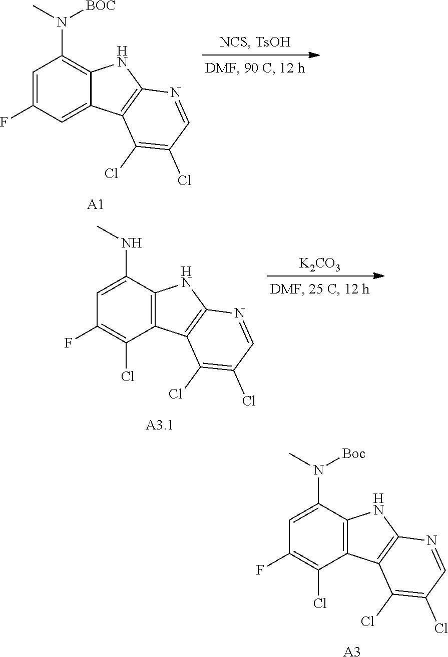

### Step (a) Preparation of 3,4,5-trichloro-6-fluoro-N-methyl-9H-pyrido[2,3-b]indol-8-amine (compound A3.1)

To a stirred solution of tert-butyl (3,4-dichloro-6-fluoro-9H-pyrido[2,3-b]indol-8-yl)(methyl)carbamate (5 g, 13.02 mmol, Intermediate A1) in DMF (150 mL) was added 1-chloropyrrolidine-2,5-dione (2.27 g, 39.06 mmol) and 4-methylbenzenesulfonic acid (4.48 mg, 26.04 mmol). The mixture was stirred at 90° C. for 12 h until LC-MS showed the starting material was completely consumed. The reaction mixture was cooled back to r.t. and concentrated in vacuo. The crude product was purified by silica gel flash chromatography (petroleum ether:EtOAc=10:1˜1:1) to give 3,4,5-trichloro-6-fluoro-N-methyl-9H-pyrido[2,3-b]indol-8-amine (3 g,71.9% yield) as a yellow solid. MS (ESI): 319.9 (M+H]⁺).

### Step (b) Preparation of tert-butyl methyl(3,4,5-trichloro-6-fluoro-9H-pyrido[2,3-b]indol-8-yl)carbamate (Intermediate A3)

To a stirred solution of 3,4,5-trichloro-6-fluoro-N-methyl-9H-pyrido[2,3-b]indol-8-amine (3 g, 9.416 mmol) and K₂CO₃ (6.5 g, 47.08 mmol) in DMF (60 mL) was added di-tert-butyl dicarbonate (6.2 g, 28.25 mmol). The mixture was stirred at 25° C. for 12 h until TLC (petroleum ether:EtOAc=10:1) and LC-MS showed the starting material was consumed completely. The reaction mixture was concentrated in vacuo to give a crude product, which was purified by silica gel flash chromatography (petroleum ether:EtOAc=20:1˜10:1) to give tert-butyl methyl(3,4,5-trichloro-6-fluoro-9H-pyrido[2,3-b]indol-8-yl)carbamate (2.1 g; 53.8% yield) as a yellow solid. MS (ESI): 418.0 ([{³⁵Cl}M+H]⁺), 317.9 {M-56}+H]⁺). ¹H NMR (400 MHz, DMSO-d6) δ ppm 9.75 (s, 1H), 8.59 (s, 1H), 7.27-7.30 (m, 1H), 3.37 (s, 3H), 1.43 (m, 9H).

### Intermediate A4

### tert-Butyl(3,4-dichloro-5-cyano-6-fluoro-9H-pyrido[2,3-b]indol-8-yl)(methyl) carbamate

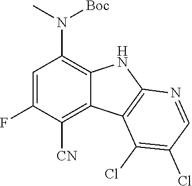 [SMILES: CN(C(=O)OC(C)(C)C)c1cc(F)c(C#N)c2c1[nH]c1ncc(Cl)c(Cl)c12]

The titled compound was synthesized according to the following scheme:

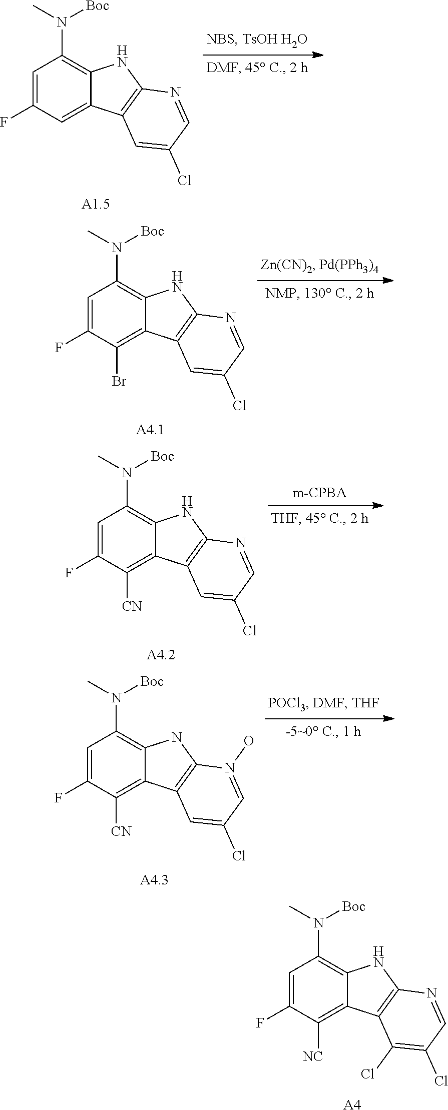

### Step (a) Preparation of tert-butyl(5-bromo-3-chloro-6-fluoro-9H-pyrido[2,3-b]indol-8-yl)(methyl)carbamate (compound A4.1)

To a solution of tert-butyl (3-chloro-6-fluoro-9H-pyrido[2,3-b]indol-8-yl) (methyl)carbamate (4.5 g, 12.86 mmol, compound A1.5) in DMF (60 mL) was added NBS (4.58 g, 25.72 mmol) and TsOH.H₂O (1.22 g, 6.43 mmol). The reaction mixture was stirred at 45° C. for 2 h before it was cooled back to r.t. and poured into aq. Na₂SO₃ solution (10%, 200 mL) and adjusted to pH=8 with aq. Na₂CO₃ solution. The mixture was stirred at 25° C. for additional 0.5 h before it was extracted with EtOAc (150 mL) three times. The combined organics were washed with satd. aq. CaCl₂ solution (100 mL) four times, brine (100 mL) three times, dried over anhy. Na₂SO₄, filtered, and concentrated in vacuo to give a crude product. It was then recrystallized in MeOH (40 mL) to give tert-butyl (5-bromo-3-chloro-6-fluoro-9H-pyrido[2,3-b]indol-8-yl)(methyl)carbamate (4.8 g, 87% yield) as a yellow solid. MS (ESI): 428.0 ([{⁷⁹Br}M+H]⁺), 430.0 ([{⁸¹Cl}M+H]⁺).

### Step (b) Preparation of tert-butyl(3-chloro-5-cyano-6-fluoro-9H-pyrido[2,3-b]indol-8-yl)(methyl)carbamate (compound A4.2)

To a solution of tert-butyl (5-bromo-3-chloro-6-fluoro-9H-pyrido[2,3-b] indol-8-yl)(methyl)carbamate (1.3 g, 3.03 mmol), Zn(CN)₂ (1.42 g, 12.13 mmol) in NMP (10 mL) was added Pd(PPh₃)₄ (350 mg, 0.303 mmol) in glovebox under argon and the resulting reaction mixture was stirred at 130° C. for 2 h under argon. After LC-MS showed the starting material was consumed completely, the reaction mixture was cooled back to r.t., and the mixture was diluted with EtOAc (200 mL) and filtered. The filtrate was washed with brine (80 mL) three times, and the organic layer was dried over anhy. Na₂SO₄, filtered, and concentrated in vacuo to give a crude product, which was recrystallized in MeOH to give tert-butyl (3-chloro-5-cyano-6-fluoro-9H-pyrido[2,3-b]indol-8-yl) (methyl)carbamate (1 g, 88% yield) as a yellow solid. MS (ESI): 375.0 ([{³⁵Cl}M+H]⁺), 377.0 ([{³⁷Cl}M+H]⁺).

### Step (c) Preparation of 8-((tert-butoxycarbonyl)(methyl)amino)-3-chloro-5-cyano-6-fluoro-9H-pyrido[2,3-b]indole 1-oxide (compound A4.3)

To a solution of tert-butyl (3-chloro-5-cyano-6-fluoro-9H-pyrido[2,3-b]indol-8-yl)(methyl)carbamate (1 g, 2.67 mmol) in TRF (10 mL) was added m-CPBA (1.08 g, 5.34 mmol, 85% wt). After the reaction mixture was degassed with nitrogen for 2 min, it was stirred at 45° C. for 2 h. After TLC (petroleum ether:EtOAc=2:1) showed the starting material was consumed completely, the reaction mixture was poured into aq. Na₂SO₃ solution (10%, 50 mL) and stirred at r.t. for 1 h. The resulting precipitate was collected by filtration and the filter cake was washed with aq. Na₂SO₃ solution (5%, 50 mL). The filter cake was dried under vacuum to give a crude product of 8-((tert-butoxycarbonyl)(methyl)amino)-3-chloro-5-cyano-6-fluoro-9H-pyrido[2,3-b]indole 1-oxide (1.0 g, 95.9% yield) as a yellow solid. MS (ESI): 391.1 ([{³⁵Cl}M+H]⁺), 393.1 ([{³⁷Cl}M+H]⁺). It was used directly in the next step without further purification.

### Step (d) Preparation of tert-butyl(3,4-dichloro-5-cyano-6-fluoro-9H-pyrido[2,3-b]indol-8-yl)(methyl)carbamate (Intermediate A4)

To a solution of 8-((tert-butoxycarbonyl)(methyl)amino)-3-chloro-5-cyano-6-fluoro-9H-pyrido[2,3-b]indole 1-oxide (1 g, 2.56 mmol) in DMF/TRF (10 mL, v/v=1:1) was added POCl₃ (4.26 g, 27.8 mmol) dropwise under ice-salt bath and the resulting reaction mixture was stirred at −5-0° C. for 1 h. After TLC (petroleum ether:EtOAc=2:1) showed the starting material was consumed completely, the reaction mixture was poured into satd. aq. NaHCO₃ solution (100 mL) at 0° C. and extracted with EtOAc (200 mL) twice. The combined organics were washed with satd. aq. NaHCO₃ solution (50 mL) twice, aq. CaCl₂ solution (1N, 50 mL) four times, and brine (50 mL) twice. The organic layer was dried over anhy. Na₂SO₄, filtered, and concentrated in vacuo to give a crude product, which was purified by silica gel flash chromatography (petroleum ether:EtOAc=100:1˜2:1, 20% DCM as additive) to give tert-butyl (3,4-dichloro-5-cyano-6-fluoro-9H-pyrido [2,3-b]indol-8-yl)(methyl)carbamate (900 mg, 85.94% yield) as a yellow solid. MS (ESI): 409.2 ([{³⁵Cl}M+H]⁺), 411.2 ([{³⁷Cl}M+H]⁺).

### Intermediate A5

### tert-Butyl(3-bromo-4-chloro-6-fluoro-9H-pyrido[2,3-b]indol-8-yl)(methyl)carbamate

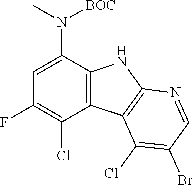 [SMILES: CN(C(=O)OC(C)(C)C)c1cc(F)c(Cl)c2c1[nH]c1ncc(Br)c(Cl)c12]

The titled compound was synthesized according to the following scheme:

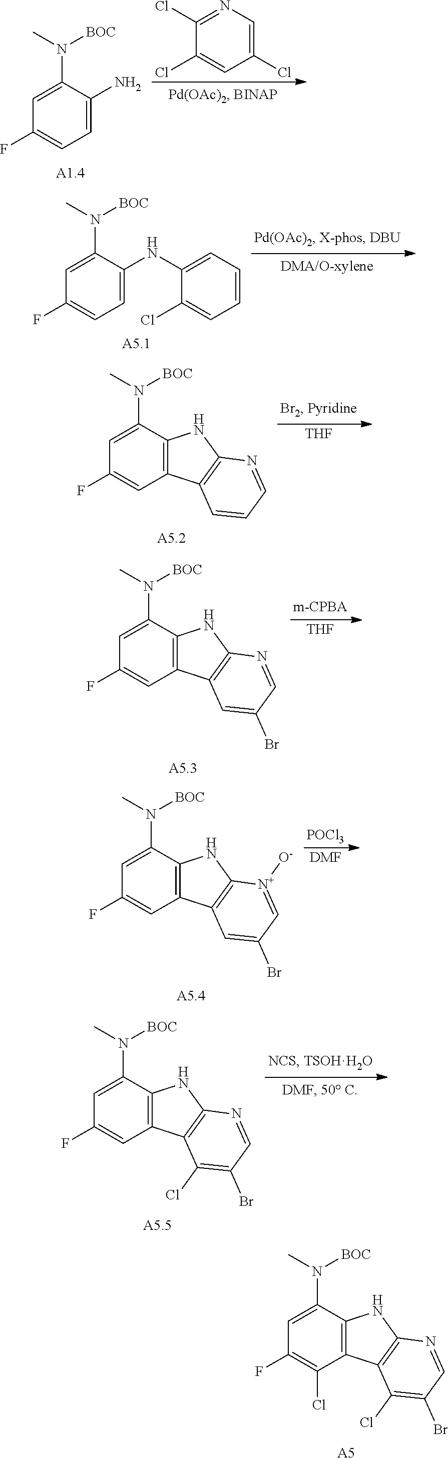

### Step (a) Preparation of tert-butyl N-(3-chloro-6-fluoro-9H-pyrido[2,3-b]indol-8-yl)-N-methyl-carbamate (compound A5.1)

To a solution of tert-butyl N-(2-amino-5-fluoro-phenyl)-N-methyl-carbamate (45.0 g, 187.3 mmol, compound A1.4) and 2,3-dichloropyridine (25.0 g, 168.6 mmol, CAS: 2402-77-9) in dioxane (800 mL) were added cesium carbonate (122.0 g, 374.6 mmol), palladium(II) acetate (4.2 g, 18.73 mmol), and BINAP (23.9 g, 37.46 mmol, CAS: 98327-87-8). The reaction mixture was continued at 120° C. for 3 h under nitrogen atmosphere. Then the reaction mixture was cooled back to room temperature, diluted with EtOAc (500 mL), and the precipitate was removed by filtration. The filtrate was concentrated in vacuo and the residue was purified by silica gel flash chromatography (2% to 5% EtOAc in petroleum ether) to give tert-butyl N-[2-[(3,5-dichloro-2-pyridyl)amino]-5-fluoro-phenyl]-N-methyl-carbamate (40 g, 67.5% yield) as a white solid. MS (ESI): 352.0 ([M+H]⁺).

### Step (b) Preparation of tert-butyl(6-fluoro-9H-pyrido[2,3-b]indol-8-yl)(methyl)carbamate (compound A5.2)

To a solution of tert-butyl N-(3-chloro-6-fluoro-9H-pyrido[2,3-b]indol-8-yl)-N-methyl-carbamate (37.5 g, 106.6 mmol) in o-xylene (32 mL) and DMA (32 mL) was added DBU (32.4 g, 213.2 mmol), Pd₂(dba)₃ (19.5 g, 21.32 mmol), and x-phos (20.3 g, 42.64 mmol) under nitrogen atmosphere. The reaction mixture was stirred at 130° C. for 3 h until LCMS showed the starting material was consumed. After cooling to 40° C., the reaction mixture was diluted with EtOAc (500 mL) and filtered to give a brown solution, which was washed with satd. aq. CaCl₂ solution (500 mL) five times, water (500 mL), and then concentrated in vacuo to give a residue. The residue was then purified by trituration (MeOH, 50 mL; and then petroleum ether, 50 mL) to give tert-butyl (6-fluoro-9H-pyrido[2,3-b]indol-8-yl)(methyl)carbamate (30 g, 89.3% yield) as a yellow solid. MS (ESI): 316.1 [M+H]⁺).

### Step (c) Preparation of tert-butyl(3-bromo-6-fluoro-9H-pyrido[2,3-b]indol-8-yl)(methyl)carbamate (compound A5.3)

To a solution of tert-butyl (6-fluoro-9H-pyrido[2,3-b]indol-8-yl)(methyl)carbamate (28 g, 88.8 mmol) in THF (500 mL) and pyridine (8.5 g, 106.6 mmol) was added Br₂ (17.0 g, 106.6 mmol) dropwise at −20° C. The resulting reaction mixture was stirred at 25° C. for 48 h until LCMS showed the starting material was. The mixture was then poured into aq. Na₂SO₃ solution (1000 mL) and stirred for 1 h before extracted by EtOAc (1000 mL) three times. Combined organics were washed with brine (1000 mL) three times, dried over anhy. Na₂SO₄, filtered, and concentrated in vacuo to give a residue, which was triturated by MeOH (500 mL) to give tert-butyl (3-bromo-6-fluoro-9H-pyrido[2,3-b]indol-8-yl)(methyl)carbamate (31 g, 88.6% yield) as a yellow solid. MS (ESI): 394.1 ([M+H]⁺).

### Step (d) Preparation of 3-bromo-8-((tert-butoxycarbonyl)(methyl)amino)-6-fluoro-9H-pyrido[2,3-b]indole 1-oxide (compound A5.4)

To a solution of tert-butyl (3-bromo-6-fluoro-9H-pyrido[2,3-b]indol-8-yl)(methyl)carbamate (29.0 g, 73.6 mmol) in TRF (500 mL) was added m-CPBA (25.4 g, 147.2 mmol), and the resulting reaction mixture was stirred at 25° C. for 2 h. After LC-MS showed the starting material was consumed, the reaction mixture was poured into aq. Na₂SO₃ solution (1500 mL, 10%) and stirred for 2 h. The mixture was filtered, and the filter cake was washed with aq. Na₂SO₃ solution (500 mL, 10%) and water (500 mL), and then dried under vacuum to give 3-bromo-8-((tert-butoxycarbonyl)(methyl)amino)-6-fluoro-9H-pyrido[2,3-b]indole 1-oxide (27 g, 89.4% yield) as a yellow solid. MS (ESI): 410.0 ([^{{79Br}}M+H]⁺), 412.0 ([^{{81Br}}M+H]⁺).

### Step (e) Preparation of tert-butyl(3-bromo-4-chloro-6-fluoro-9H-pyrido[2,3-b]indol-8-yl)(methyl)carbamate (compound A5.5)

To a solution of 3-bromo-8-((tert-butoxycarbonyl)(methyl)amino)-6-fluoro-9H-pyrido[2,3-b]indole 1-oxide (26.0 g, 63.4 mmol) in TRF (250 mL) and DMF (250 mL) was added POCl₃ (131.75 g, 859.2 mmol) at −5 to 0° C. And the reaction mixture was stirred at 0° C. for 3 h. After LC-MS and TLC (petroleum ether:EtOAc=3:1) showed the starting material was consumed, the reaction mixture was then poured into aq. Na₂CO₃solution (3 L, IN) and extracted by EtOAc (1 L) three times. Combined organics were washed with aq.aq. CaCl₂ solution (500 mL, IN) five times, brine (500 mL) three times. The organic layer was dried over anhy. Na₂SO₄ filtered, and concentrated in vacuo to give a residue, which was triturated in MeOH (400 mL) to give tert-butyl (3-bromo-4-chloro-6-fluoro-9H-pyrido[2,3-b]indol-8-yl)(methyl)carbamate (23 g, 84.6% yield) as a yellow solid. MS (ESI): 430.0 ([M+H]⁺). ¹H NMR (400 MHz, DMSO-d6) δppm: 12.54 (s, 1H), 8.70 (s, 1H), 8.00-8.06 (m, 1H), 7.47 (dd, J=10.3, 2.5 Hz, 1H), 3.25 (s, 3H), 1.17-1.29 (m, 9H).

### Step (f) Preparation of tert-Butyl(3-bromo-4,5-dichloro-6-fluoro-9H-pyrido[2,3-b]indol-8-yl)(methyl) carbamate (compound A5)

To a solution of tert-butyl (3-bromo-4-chloro-6-fluoro-9H-pyrido[2,3-b]indol-8-yl)(methyl)carbamate (2.5 g, 5.83 mmol) in DMF (20 mL) was added TsOH.H₂O (700 mg, 3.68 mmol) and NCS (1.70 g, 11.46 mmol), and the resulting mixture was stirred at 50° C. for 2 h. After cooled back to r.t., the reaction mixture was poured into water (200 mL) and extracted with EtOAc (100 mL) three times. Combined organics were washed with brine (100 mL) twice, dried over anhy. Na₂SO₄, filtered, and concentrated in vacuo to give a residue, which was purified by silica gel chromatography (petroleum ether/EtOAc, a gradient from 20/1 to 5/1) to give tert-butyl (3-bromo-4,5-dichloro-6-fluoro-9H-pyrido[2,3-b]indol-8-yl)(methyl)carbamate (1.80 g, 66.6% yield) as a white solid. MS obsd. (Est) [{³⁵Cl×2+⁸¹Br & ³⁵Cl+³⁷Cl+⁷⁹Br}(M+H)⁺]: 464.0. ¹H NMR (400 MHz, DMSO-d6) δppm: 12.94 (s, 1H), 8.78 (s, 1H), 7.72-7.70 (d, 1H), 3.22 (s, 1H), 1.49 (s, 3H), 1.25 (s, 6H).

### Intermediate B1

### Ethyl 1-methyl-4-oxo-6-(4,4,5,5-tetramethyl-1,3,2-dioxaborolan-2-yl)-1,4-dihydro-1,8-naphthyridine-3-carboxylate

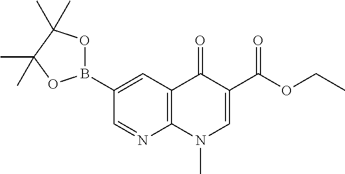 [SMILES: CCOC(=O)c1cn(C)c2ncc(B3OC(C)(C)C(C)(C)O3)cc2c1=O]

The titled compound was synthesized according to the following scheme:

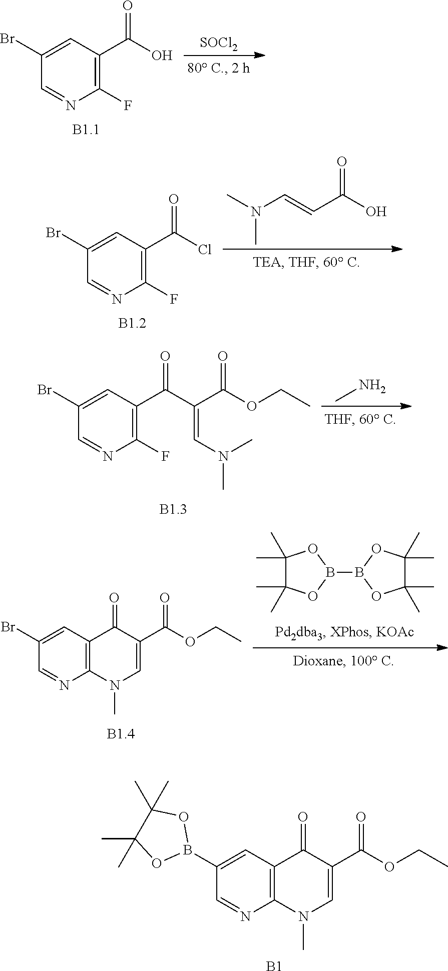

### Step (a) Preparation of 5-bromo-2-fluoronicotinoyl chloride (compound B1.2)

A solution of 5-bromo-2-fluoro-pyridine-3-carboxylic acid (30 g, 137 mmol, compound B1.1) in SOCl₂ (150 mL) was stirred at 80° C. for 2 h. After cooled back to r.t., the reaction mixture was concentrated under reduced pressure to give 5-bromo-2-fluoronicotinoyl chloride (31.2 g, 96% yield) as a crude product. It was used directly in next step without further purification.

### Step (b) Preparation of ethyl 2-(5-bromo-2-fluoronicotinoyl)-3-(dimethylamino)acrylate (compound B1.3)

To a mixture of 5-bromo-2-fluoronicotinoyl chloride (31.2 g, 131.7 mmol) and Et₃N (26.3 g, 263.4 mmol) in TRF (300 mL) was added 3-(dimethylamino)prop-2-enoic acid (26.3 g, 197.6 mmol), and the reaction mixture was stirred at 60° C. for 2 h. After cooled back to r.t., the mixture was poured into water (300 mL), and extracted with EtOAc (200 mL) three times. Combined organics were washed with brine (100 mL) twice, dried over anhy. Na₂SO₄, filtered, and concentrated in vacuo to give a crude product of ethyl 2-(5-bromo-2-fluoronicotinoyl)-3-(dimethylamino)acrylate (42.3 g, 93.4% yield). It was used directly in the next step without further purification. MS (ESI): 346.9 ([{⁸¹Br}M+H]⁺), 344.9 ([{⁷⁹Br}M+H]⁺).

### Step (c) Preparation of ethyl 6-bromo-1-methyl-4-oxo-1,4-dihydro-1,8-naphthyridine-3-carboxylate (compound B1.4)

A mixture solution of ethyl 2-(5-bromo-2-fluoronicotinoyl)-3-(dimethylamino)acrylate (32.0 g, 92.71 mmol, compound B1.3) and methylamine (400 mL, 300 mmol, 2M in TRF) was stirred at 60° C. for 0.5 h. After TLC (petroleum ether/EtOAc=2:1) showed the started material was consumed, the mixture was evaporated to dryness under the reduced pressure. The residue was re-dissolved in EtOAc (300 mL) and filtered. The filtrate was concentrated in vacuo to give crude product, which was recrystallized in MeOH (200 mL) to give ethyl 6-bromo-1-methyl-4-oxo-1,4-dihydro-1,8-naphthyridine-3-carboxylate (23 g, 79.8% yield) as a white solid. MS (ESI): 311.0({⁸⁰Br}M+H)⁺, 313.0({⁸²Br}M+H)⁺.

### Step (d) Preparation of ethyl 1-methyl-4-oxo-6-(4,4,5,5-tetramethyl-1,3,2-dioxaborolan-2-yl)-1,4-dihydro-1,8-naphthyridine-3-carboxylate (Intermediate B1)

To a mixture solution of ethyl 6-bromo-1-methyl-4-oxo-1,4-dihydro-1,8-naphthyridine-3-carboxylate (20 g, 64.28 mmol) and B₂Pin₂ (32.7 g, 128.56 mmol) in dioxane (200 mL) was added KOAc (18.9 g, 104.13 mmol), Pd2(dba)3 (5.89 g, 6.43 mmol), and x-phos (6.13 g, 12.86 mmol) at 0° C. under N₂. The resulting mixture was heated to 100° C. and stirred for 2 h. After LC-MS showed the started material was consumed, the mixture was poured into DCM (2 L) and filtered. The filtrate was concentrated in vacuo to give a crude product, which was recrystallized in petroleum ether/MTBE (500 mL, v/v=1:1) to give ethyl 1-methyl-4-oxo-6-(4,4,5,5-tetramethyl-1,3,2-dioxaborolan-2-yl)-1,4-dihydro-1,8-naphthyridine-3-carboxylate (20.9 g, 90.6% yield) as a yellow solid. MS (ESI): 277.0 ([M-82+H]⁺). ¹H NMR (400 MHz, DMSO-d6) δ: 8.89˜8.89 (d, J=1.6 Hz, 1H), 8.82 (s, 1H), 8.69˜8.69 (d, J=1.2 Hz, 1H), 4.21˜4.27 (q, J=7.2 Hz, 2H), 3.92 (s, 3H), 1.23˜1.31 (m, 15H).

### Intermediate B2

### Ethyl 1-ethyl-4-oxo-6-(4,4,5,5-tetramethyl-1,3,2-dioxaborolan-2-yl)-1,4-dihydro-1,8-naphthyridine-3-carboxylate

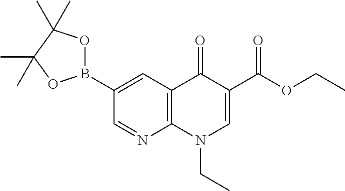 [SMILES: CCOC(=O)c1cn(CC)c2ncc(B3OC(C)(C)C(C)(C)O3)cc2c1=O]

In analogy to the synthesis of ethyl 1-methyl-4-oxo-6-(4,4,5,5-tetramethyl-1,3,2-dioxaborolan-2-yl)-1,4-dihydro-1,8-naphthyridine-3-carboxylate (Intermediate B1), the title compound was prepared by replacing methylamine with ethylamine in step (c). MS (ESI): 291.1 ([M-82+H]⁺).

### Intermediate C1

### Cis-tert-butyl(3-(hexahydropyrrolo[3,4-b]pyrrol-5(1H)-yl)propyl)carbamate

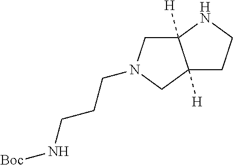 [SMILES: CC(C)(C)OC(=O)NCCCN1C[C@@H]2CCN[C@@H]2C1]

The titled compound was synthesized according to the following scheme:

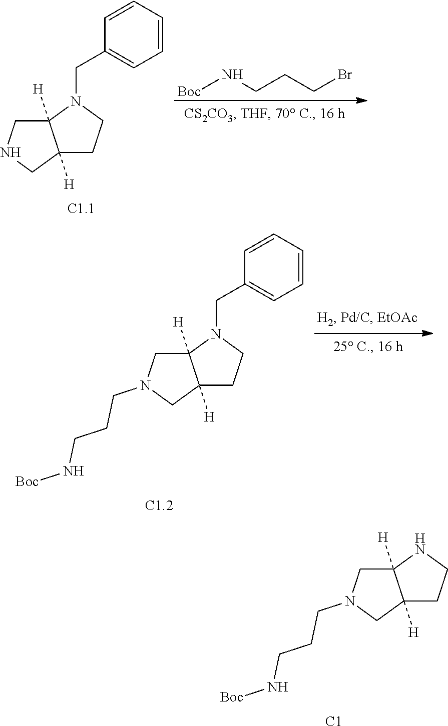 [SMILES: c1ccc(CN2CC[C@H]3CNC[C@H]32)cc1 ; CC(C)(C)OC(=O)NCCCBr ; CC(C)(C)OC(=O)NCCCN1C[C@@H]2CCN(Cc3ccccc3)[C@@H]2C1 ; CC(C)(C)OC(=O)NCCCN1C[C@@H]2CCN[C@@H]2C1 ; "C1"]

### Step (a) Preparation of cis-tert-butyl(3-(1-benzylhexahydropyrrolo[3,4-b]pyrrol-5(1H)-yl)propyl)carbamate (compound C1.2)

To a solution of cis-1-benzyloctahydropyrrolo[3,4-b]pyrrole (400 mg, 1.98 mmol, CAS: 132414-50-7) in ACN (10 mL) was added cesium carbonate (1288 mg, 3.95 mmol), 3-bromopropan-1-ol (564 mg, 2.37 mmol) in one portion under N₂. The reaction mixture was heated to 70° C. for 16 h until TLC showed the starting material was consumed. The mixture was cooled back to r.t., diluted with water (20 mL), and extracted by EtOAc (20 mL) three times. Combined organics were washed with brine (50 mL), dried over anhy. Na₂SO₄, filtered, and concentrated in vacuo to give a crude product, which was purified by prep-HPLC (TFA as additive) to give cis-tert-butyl N-[3-(1-benzyl-2,3,3a,4,6,6a-hexahydropyrrolo[2,3-c]pyrrol-5-yl)propyl]carbamate (395 mg, 55.6% yield) as yellow oil. MS (ESI): 360.2 (M+H)⁺.

### Step (b) Preparation of tert-butyl(3-(hexahydropyrrolo[3,4-b]pyrrol-5(1H)-yl)propyl)carbamate (compound C1)

To a solution of cis-tert-butyl N-[3-(1-benzyl-2,3,3a,4,6,6a-hexahydropyrrolo[2,3-c]pyrrol-5-yl)propyl]carbamate (395.0 mg, 1.15 mmol) in EtOAc (20 mL) was added Pd/C (248 mg, 0.230 mmol) under N₂. After the suspension was degassed under vacuum and purged with H₂ several times, the reaction mixture was stirred at 25° C. for 16 h under H₂ (15 psi). After TLC showed the reaction was completed, the mixture was filtered through Celite and the filter cake was washed with EtOAc (20 mL) twice. Combined organic phase was concentrated in vacuo to give a crude product of cis-N-[3-(2,3,3a,4,6,6a-hexahydro-1H-pyrrolo[2,3-c]pyrrol-5-yl)propyl]-2,2-dimethyl-propanamide (215 mg, 73.8% yield) as yellow oil. It was used directly in the next step without further purification.

### Intermediate C2

### Cis-tert-butyl 5-azaspiro[2.5]octan-1-ylcarbamate

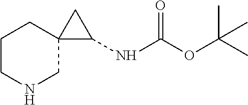

The titled compound was synthesized according to the following scheme:

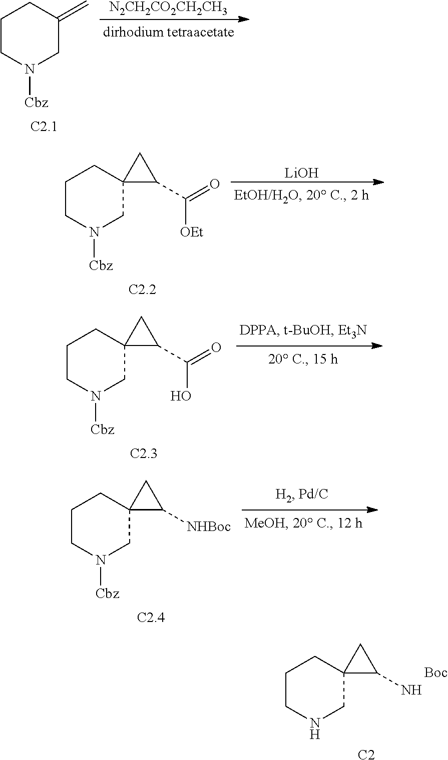

### Step (a) Preparation of cis-5-benzyl 1-ethyl 5-azaspiro[2.5]octane-1,5-dicarboxylate (compound C2.2)

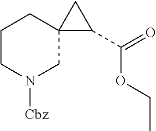

To a solution of benzyl 3-methylenepiperidine-1-carboxylate (5000.0 mg, 21.62 mmol, CAS:138163-15-2) in DCM (10 mL) was added rhodium(II) acetate dimer (955.5 mg, 2.16 mmol, CAS:15956-28-2) under N₂. After ethyl diazoacetate (9866.4 mg, 86.47 mmol) was added at 0° C. under ice-water bath, the resulting reaction mixture was allowed to warm up and stirred at 25° C. for 4 h. After TLC (petroleum ether:EtOAc=5:1) showed the starting material was consumed, the mixture was then washed with water (10 mL). The separated organic layer was dried over anhy. Na₂SO₄, filtered, and concentrated in vacuo to give a crude product, which was purified by silica gel flash chromatography (petroleum ether/ethyl acetate=5:1) to give 5-benzyl-2-ethyl 5-azaspiro[2.5]octane-2,5-dicarboxylate (5 g, 72.9% yield) as yellow oil. MS (ESI): 318.2 ([M+H]⁺).

### Step (b) Preparation of cis-5-((benzyloxy)carbonyl)-5-azaspiro[2.5]octane-1-carboxylic acid (compound C2.3)

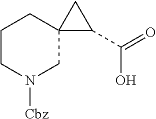

To a solution of cis-5-benzyl-2-ethyl 5-azaspiro[2.5]octane-2,5-dicarboxylate (5000.0 mg, 15.75 mmol) in EtOH (10 mL) and water (2.0 mL) was added LiOH (6459.1 mg, 157.54 mmol). The resulting reaction mixture was stirred at 25° C. for 15 h until TLC (petroleum ether:EtOAc=5:1) showed the reaction was completed. The reaction mixture was acidified with aq. HCl solution (1N) to pH=6 and extracted with DCM (50 mL) three times. Combined organics were washed with brine, dried over anhy. Na₂SO₄, filtered, and concentrated in vacuo to give a crude product of 5-benzyloxycarbonyl-5-azaspiro[2.5]octane-2-carboxylic acid (2 g, 43.9% yield) as yellow oil. It was used directly in the next step without further purification. MS (ESI): 290.2 ([M+H]⁺).

### Step (c) Preparation of cis-benzyl 1-((tert-butoxycarbonyl)amino)-5-azaspiro[2.5]octane-5-carboxylate (compound C2.4)

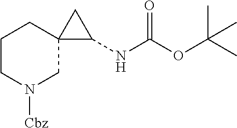

To a solution of 5-benzyloxycarbonyl-5-azaspiro[2.5]octane-2-carboxylic acid (2 g, 6.91 mmol) in toluene (1 mL) and tert-butanol (1 mL) was added DPPA (2.8 g, 10.37 mmol) and Et₃N (2.89 mL, 20.74 mmol). The resulting reaction mixture was stirred at 70° C. for 15 h until LC-MS showed the reaction was completed. After cooled back to r.t., the mixture was concentrated in vacuo to give a crude product, which was purified by silica gel flash chromatography (petroleum ether:ethyl acetate=5:1) to give cis-benzyl 2-(tert-butoxycarbonylamino)-5-azaspiro[2.5]octane-5-carboxylate (1.2 g, 48.2% yield) as yellow oil. MS (ESI): 361.2 ([M+H]⁺).

### Step (d) Preparation of cis-tert-butyl 5-azaspiro[2.5]octan-1-ylcarbamate (Intermediate C2)

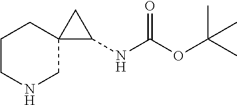

To a solution of cis-benzyl 2-(tert-butoxycarbonylamino)-5-azaspiro[2.5]octane-5-carboxylate (1.2 g, 3.33 mmol) in MeOH (50 mL) was added Pd/C (120 mg, 3.33 mmol) under N₂. After the suspension was degassed under vacuum and purged with H₂ several times, the reaction mixture was stirred at 25° C. for 16 h under a H₂ balloon. After LC-MS showed the starting material was consumed, the mixture was filtered through Celite, and the filtrate was concentrated in vacuo to give a crude product of cis-tert-butyl N-(5-azaspiro[2.5]octan-2-yl)carbamate (600 mg, 79.6% yield) as a white solid. MS (ESI): 227.2 ([M+H]⁺). It was used directly in the next step without further purification.

### Example 1.01

### 6-[4-[3-(1-Aminocyclopropyl)pyrrolidin-1-yl]-5,6-difluoro-8-(methylamino)-9H-pyrido[2,3-b]indol-3-yl]-1-methyl-4-oxo-1,8-naphthyridine-3-carboxylic acid

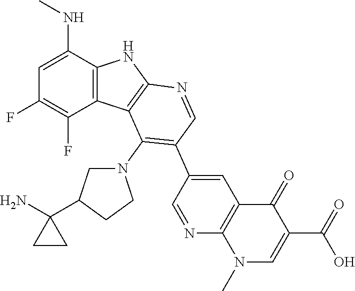 [SMILES: CNc1cc(F)c(F)c2c1[nH]c1ncc(-c3cnc4c(c3)c(=O)c(C(=O)O)cn4C)c(N3CCC(C4(N)CC4)C3)c12]

### Step (a) Preparation of tert-butyl N-[4-[3-[1-(tert-butoxycarbonylamino)cyclopropyl]pyrrolidin-1-yl]-3-chloro-5,6-difluoro-9H-pyrido[2,3-b]indol-8-yl]-N-methyl-carbamate

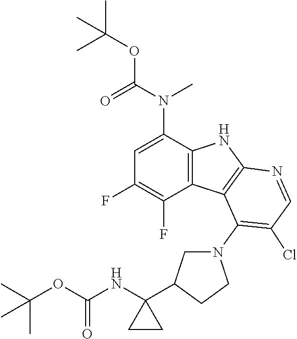 [SMILES: CN(C(=O)OC(C)(C)C)c1cc(F)c(F)c2c1[nH]c1ncc(Cl)c(N3CCC(C4(NC(=O)OC(C)(C)C)CC4)C3)c12]

To a solution of tert-butyl N-(3,4-dichloro-5,6-difluoro-9H-pyrido[2,3-b]indol-8-yl)-N-methyl-carbamate (Intermediate A2, 2.80 g, 6.96 mmol, as the “CORE” in table 1) and DIPEA (11.69 g, 90.48 mmol) in DMSO (6.0 mL) was added tert-butyl N-(1-pyrrolidin-3-ylcyclopropyl)carbamate (3.15 g, 13.92 mmol, as the “AMINE” in table 1), and the resulting reaction solution was stirred at 130° C. for 12 h. After it was cooled back to r.t., the mixture was poured into water (150 mL) and extracted with EtOAc (100 mL) two times. The combined organics was washed with brine (100 mL) three times, dried over anhy. Na₂SO₄, filtered, and concentrated in vacuo to give a crude product, which was purified by silica gel flash chromatography (5% to 25% EtOAc in petroleum ether) to give tert-butyl N-[4-[3-[1-(tert-butoxycarbonylamino)cyclopropyl]pyrrolidin-1-yl]-3-chloro-5,6-difluoro-9H-pyrido[2,3-b]indol-8-yl]-N-methyl-carbamate (0.82 g, 20% yield) as a yellow solid. MS (ESI): 592.4 ([{³⁵Cl}M+H]⁺), 594.3 ([{³⁷Cl}M+H]⁺).

### Step (b) Preparation of ethyl 6-[4-[3-[1-(tert-butoxycarbonylamino)cyclopropyl]pyrrolidin-1-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylate

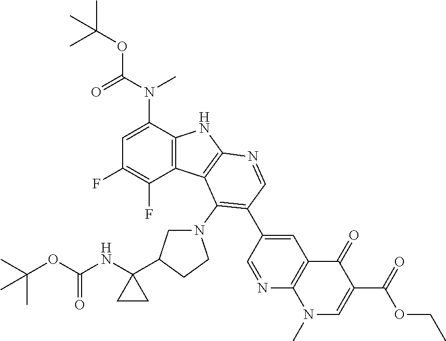 [SMILES: CCOC(=O)c1cn(C)c2ncc(-c3cnc4[nH]c5c(N(C)C(=O)OC(C)(C)C)cc(F)c(F)c5c4c3N3CCC(C4(NC(=O)OC(C)(C)C)CC4)C3)cc2c1=O]

A solution of mixture of tert-butyl N-[4-[3-[1-(tert-butoxycarbonylamino)cyclopropyl]pyrrolidin-1-yl]-3-chloro-5,6-difluoro-9H-pyrido[2,3-b]indol-8-yl]-N-methyl-carbamate (100 mg, 0.17 mmol), ethyl 1-methyl-4-oxo-6-(4,4,5,5-tetramethyl-1,3,2-dioxaborolan-2-yl)-1,8-naphthyridine-3-carboxylate (93 mg, 0.34 mmol) (Intermediate B1, as the “BORONIC REAGENT” in table 1), K₃PO₄ (108 mg, 0.51 mmol) and Pd-Ad₂nBuP Biphenyl Precat (26.6 mg, 0.034 mmol, CAS #:1310584-14-5, as the “CATALYST” in table 1) in dioxane/H₂O (3.8 mL, v/v=4:1 as the “SOLVENT” in table 1) was stirred at 60° C. under argon for 70 h. After it was cooled back to r.t., the reaction mixture was diluted with EtOAc (50 mL), and washed with brine (30 mL) two times. The combined organics was dried over anhy. Na₂SO₄, filtered, and concentrated in vacuo to give a crude product, which was purified by silica gel flash chromatography (EtOAc:hexane=2:1) to give ethyl 6-[4-[3-[1-(tert-butoxycarbonylamino)cyclopropyl]pyrrolidin-1-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylate (53 mg, 40% yield) as a yellow solid. MS (ESI): 788.4 ([M+H]⁺).

### Step (c) Preparation of 6-[4-[3-[1-(tert-butoxycarbonylamino)cyclopropyl]pyrrolidin-1-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylic acid

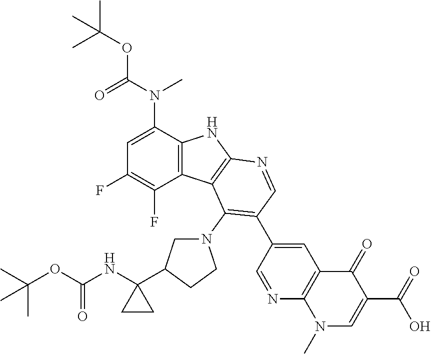 [SMILES: CN(C(=O)OC(C)(C)C)c1cc(F)c(F)c2c1[nH]c1ncc(-c3cnc4c(c3)c(=O)c(C(=O)O)cn4C)c(N3CCC(C4(NC(=O)OC(C)(C)C)CC4)C3)c12]

To a solution of ethyl 6-[4-[3-[1-(tert-butoxycarbonylamino)cyclopropyl]pyrrolidin-1-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylate (50 mg, 0.064 mmol) in MeOH (5 mL) was added aq. LiOH solution (0.5 ml, 1N) and the mixture was stirred at 15° C. for 16 hr. Afterwards, the mixture was concentrated in vacuo to obtain a crude product of 6-[4-[3-[1-(tert-butoxycarbonylamino)cyclopropyl]pyrrolidin-1-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylic acid (48.6 mg) as a yellow solid. MS (ESI): 760.3 ([M+H]⁺). It was used directly in the next step without further purification.

### Step (d) Preparation of 6-[4-[3-(1-aminocyclopropyl)pyrrolidin-1-yl]-5,6-difluoro-8-(methylamino)-9H-pyrido[2,3-b]indol-3-yl]-1-methyl-4-oxo-1,8-naphthyridine-3-carboxylic acid (Ex. 1.01)

To a solution of 6-[4-[3-[1-(tert-butoxycarbonylamino)cyclopropyl]pyrrolidin-1-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylic acid (48.6 mg, 0.064 mmol) in DCM (3 mL) was added TFA (0.5 mL), and the reaction mixture was stirred at 15° C. for 1 h. After LC-MS showed the starting material was consumed, the mixture was concentrated in vacuo to give a crude product, which was purified by pre-HPLC (0.5% NH₃.H₂O as additive) to give 6-[4-[3-(1-aminocyclopropyl)pyrrolidin-1-yl]-5,6-difluoro-8-(methylamino)-9H-pyrido[2,3-b]indol-3-yl]-1-methyl-4-oxo-1,8-naphthyridine-3-carboxylic acid (5.2 mg, 14% yield) as a yellow solid. MS (ESI): 560.3 ([M+H]⁺). ¹H NMR (400 MHz, MeOH-d4) δ 8.8-9.0 (m, 2H), 8.64 (s, 1H), 8.17 (s, 1H), 6.48 (br dd, 1H, J=6.5, 13.0 Hz), 4.06 (s, 3H), 3.38 (m, 1H), 2.9-3.1 (m, 2H), 2.88 (s, 3H), 2.43 (br d, 2H, J=12.5 Hz), 1.7-1.9 (m, 1H), 1.55 (m, 2H), 1.19 (s, 1H), 0.55 (br d, 2H, J=9.8 Hz).

The following examples were prepared in analogy to Example 1.01, replacing tert-butyl N-(3,4-dichloro-5,6-difluoro-9H-pyrido[2,3-b]indol-8-yl)-N-methyl-carbamate (Intermediate A2) with the “CORE” in step (a), tert-butyl N-(1-pyrrolidin-3-ylcyclopropyl)carbamate with the “AMINE” in step (a), dioxane/H₂O with the “SOLVENT” in step (b), Pd-Ad₂nBuP Biphenyl Precat with the “CATALYST” in step (b) and ethyl 1-methyl-4-oxo-6-(4,4,5,5-tetramethyl-1,3,2-dioxaborolan-2-yl)-1,8-naphthyridine-3-carboxylate with the “BORONIC REAGENT” in step (b) by the reagents indicated in Table 1.

TABLE 1
Compound synthesis and characterization

| | | CORE, AMINE, | |
|---|---|---|---|
| | | BORONIC REAGENT, | |
| | | CATALYST and | |
| No. | Compound Name and Structure | SLOVENT | ¹H NMR and MS (ESI) |
| 1.02 | 6-[4-(7-Amino-2- | CORE: Intermediate A2 | ¹H NMR (400 MHz, |
| | azaspiro[3.3]heptan-2-yl)- | AMINE: tert-Butyl N- | MeOH-d4) δ 9.00 (br s, |
| | 5,6-difluoro-8- | (2-azaspiro[3.3]heptan-7- | 1H), 8.91 (br s, 1H), 8.53 |
| | (methylamino)-9H- | yl)carbamate | (br s, 1H), 8.01 (br s, 1H), |
| | pyrido[2,3-b]indol-3-yl]-1- | BORONIC REAGENT: | 6.40 (br s, 1H), 4.06 (s, 3H), |
| | methyl-4-oxo-1,8- | Intermediate B1 | 3.81-3.46 (m, 5H), 2.88 (s, |
| | naphthyridine-3-carboxylic | CATALYST: | 3H), 1.97-1.89 (m, 2H), |
| | acid | cataCXium A-Pd-G2 | 1.61 (m, 1H), 1.19 (m, 1H). |
| | 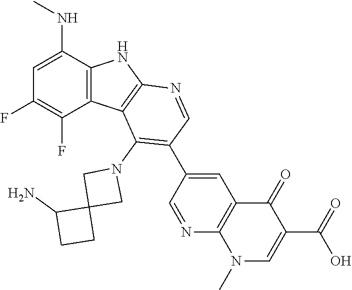 [SMILES: CNc1cc(F)c(F)c2c1[nH]c1ncc(-c3cnc4c(c3)c(=O)c(C(=O)O)cn4C)c(N3CC4(CCC4N)C3)c12] | (Sigma-Aldrich, Catalog #: 761311) SOLVENT: Dioxane/H₂O | MS (ESI): 546.2 ([M + H]⁺). |
| 1.03 | 6-[4-(3-Aminopyrrolidin-1- | CORE: Intermediate A2 | ¹H NMR (400 MHz, |
| | yl)-5,6-difluoro-8- | AMINE: tert-Butyl | MeOH-d4) δ 9.13 (br. 2 H), |
| | (methylamino)-9H- | pyrrolidin-3-ylcarbamate | 8.80 (br. 1 H), 8.26 (br. 1 |
| | pyrido[2,3-b]indol-3-yl]-1- | BORONIC REAGENT: | H), 6.62 (br. 1 H), 4.78 (br. |
| | ethyl-4-oxo-1,8- | Intermediate B2 | 1 H), 3.75-3.95 (m, 3 H), |
| | naphthyridine-3-carboxylic | CATALYST: | 3.50 (br. 3 H), 3.35 (br. 2 |
| | acid | Pd₂(dba)₃; x-Phos | H), 2.99 (br. 4 H), 2.40 (br. |
| | 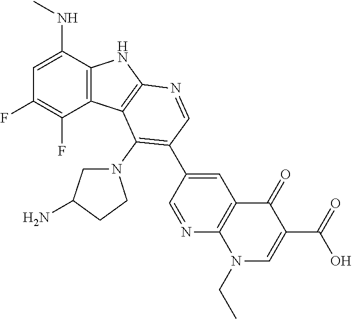 [SMILES: CCn1cc(C(=O)O)c(=O)c2cc(-c3cnc4[nH]c5c(NC)cc(F)c(F)c5c4c3N3CCC(N)C3)cnc21] | SOLVENT: Dioxane/H₂O | 1 H), 2.03 (br. 1 H), 1.61 (br. 3 H). MS (ESI): 534.2 ([M + H]⁺). |
| 1.04 | 6-(4-(3- | CORE: Intermediate A2 | ¹H NMR (400 MHz, |
| | (Aminomethyl)pyrrolidin- | AMINE: tert-Butyl | MeOH-d4) δ 9.16 (d, |
| | 1-yl)-5,6-difluoro-8- | (pyrrolidin-3- | J = 10.415 Hz, 2 H), 8.81 (br. |
| | (methylamino)-9H- | ylmethyl)carbamate | 1 H), 8.21 (br. 1 H), 6.59 |
| | pyrido[2,3-b]indol-3-yl)-1- | BORONIC REAGENT: | (dd, J = 12.674, 5.773 Hz, 1 |
| | ethyl-4-oxo-1,4-dihydro- | Intermediate B2 | H), 4.77 (d, J = 6.023 Hz, 2 |
| | 1,8-naphthyridine-3- | CATALYST: | H), 3.76 (br. 1 H), 3.46 (d, |
| | carboxylic acid | Pd₂(dba)₃; x-Phos | J = 8.031 Hz, 2 H), 3.28 (br. |
| | 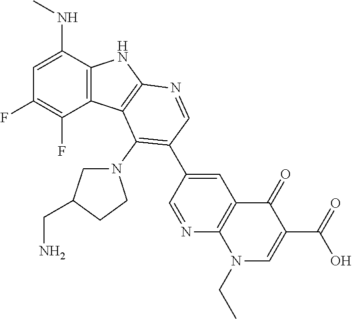 [SMILES: CCn1cc(C(=O)O)c(=O)c2cc(-c3cnc4[nH]c5c(NC)cc(F)c(F)c5c4c3N3CCC(CN)C3)cnc21] | SOLVENT: Dioxane/H₂O | 2 H), 2.98 (br. 5 H), 2.54 (br. 1 H), 2.14 (br. 1 H), 1.71 (br. 1 H), 1.60 (t, J = 6.462 Hz, 3 H). MS (ESI): 548.3 ([M + H]⁺). |
| 1.05 | 6-(4-(3-Amino-3- | CORE: Intermediate A2 | ¹H NMR (400 MHz, |
| | (trifluoromethyl)pyrrolidin- | AMINE: Benzyl (3- | DMSO-d6) δ 11.91 (s, 1 H), |
| | 1-yl)-5,6-difluoro-8- | (trifluoromethyl)pyrrolidin- | 9.31 (s, 1 H), 9.13~9.14 (d, |
| | (methylamino)-9H- | 3-yl)carbamate | J = 2.259 Hz, 1 H), 8.71-8.71 |
| | pyrido[2,3-b]indol-3-yl)-1- | BORONIC REAGENT: | (d, J = 2.259 Hz, 1 H), 8.21 |
| | methyl-4-oxo-1,4-dihydro- | Intermediate B1 | (s, 1 H), 6.60~6.65 (dd, |
| | 1,8-naphthyridine-3- | CATALYST: Pd- | J = 13.678, 6.274 Hz, 1 H), |
| | carboxylic acid | Ad₂nBuP Biphenyl | 4.18 (s, 3 H), 2.91 (s, 5 H), |
| | 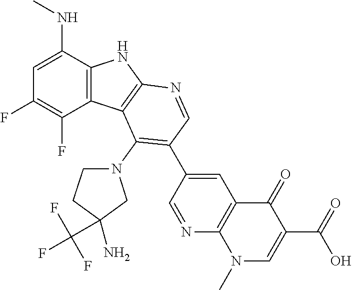 [SMILES: CNc1cc(F)c(F)c2c1[nH]c1ncc(-c3cnc4c(c3)c(=O)c(C(=O)O)cn4C)c(N3CCC(N)(C(F)(F)F)C3)c12] | SOLVENT: Dioxane/H₂O | 2.68 (br s, 1 H), 2.34~2.51 (m, 2 H), 2.03 (br s, 1 H). MS (ESI): 588.3 ([M + H]⁺). |
| 1.06 | 6-[4-[1-(Aminomethyl)-3- | CORE: Intermediate A2 | ¹H NMR (400 MHz, |
| | azabicyclo[3.1.0]hexan-3- | AMINE: Benzyl (3- | DMSO-d6) δ ppm: 12.03 (s, |
| | yl]-5,6-difluoro-8- | azabicyclo[3.1.0]hexan- | 1H), 9.29 (s, 1H), 9.08-9.02 |
| | (methylamino)-9H- | 1-ylmethyl)carbamate | (d, 1H), 8.71-8.65 (d, 1H), |
| | pyrido[2,3-b]indol-3-yl]-1- | BORONIC REAGENT: | 8.48 (s, 1H), 7.79-7.65 (m, |
| | methyl-4-oxo-1,8- | Intermediate B1 | 3H), 6.71-6.60 (m, 1H), |
| | naphthyridine-3-carboxylic | CATALYST: Pd- | 4.17-4.16 (m, 3H), 3.61- |
| | acid | Ad₂nBuP Biphenyl | 3.45 (m, 2H), 3.26-3.11 (m, |
| | 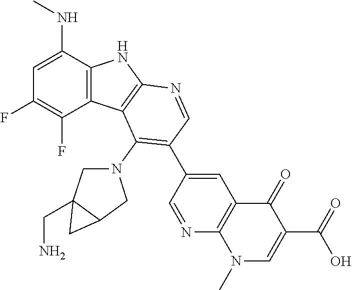 [SMILES: CNc1cc(F)c(F)c2c1[nH]c1ncc(-c3cnc4c(c3)c(=O)c(C(=O)O)cn4C)c(N3CC4CC4(CN)C3)c12] | Precat (CAS#: 1375477- 29-4) SOLVENT: THF/H₂O | 2H), 3.03-2.96 (m, 1H), 2.91 (s, 3H), 2.87-2.77 (m, 1H), 1.70-1.63 (m, 1H), 0.76-0.69 (m, 1H), 0.52 (m, 1H). MS (ESI): 546.4 ([M + H] +). |
| 1.07 | cis-6-[5,6-Difluoro-8- | CORE: Intermediate A2 | ¹H NMR (400 MHz, |
| | (methylamino)-4-[5-(3- | AMINE: C1 | DMSO-d6) δ ppm: 11.65- |
| | aminopropyl)-2,3,3a,4,6,6a- | BORONIC REAGENT: | 11.84 (m, 1H), 9.22 (s, 1H), |
| | hexahydropyrrolo[2,3- | Intermediate B1 | 9.10 (d, 1H), 8.72 (d, 1H), |
| | c]pyrrol-1-yl]-9H- | CATALYST: Pd- | 8.26 (s, 1H), 6.58-6.66 (m, |
| | pyrido[2,3-b]indol-3-yl]-1- | Ad₂nBuP Biphenyl | 1H), 4.21 (s, 4H), 3.49-3.57 |
| | methyl-4-oxo-1,8- | Precat (CAS#: 1375477- | (m, 1H), 3.33-3.46 (m, 5H), |
| | naphthyridine-3-carboxylic | 29-4) | 2.95 (s, 3H), 2.81 (s, 2H), |
| | acid | SOLVENT: THF/H₂O | 2.55-2.58 (m, 1H), 2.05- |
| | 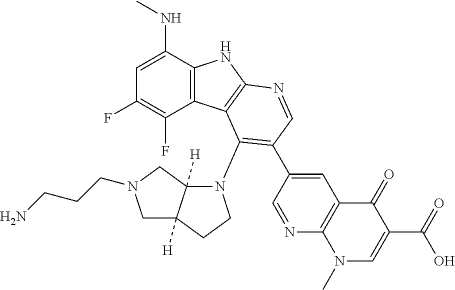 [SMILES: CNc1cc(F)c(F)c2c1[nH]c1ncc(-c3cnc4c(c3)c(=O)c(C(=O)O)cn4C)c(N3CC[C@H]4CN(CCCN)C[C@H]43)c12] | | 2.20 (m, 2H), 1.81 (s, 4H). MS (ESI): 603.2 ([M + H] ⁺). |
| 1.08 | 6-[5,6-Difluoro-8- | CORE: Intermediate A2 | ¹H NMR (400 MHz, |
| | (methylamino)-4-[rel- | AMINE: tert-butyl N- | DMSO-d6) δ ppm: 1.97 (br |
| | (1S,5R)-6-amino-3- | [rel-(1S,5R)-3- | s, 2 H), 2.19 (br s, 1 H), |
| | azabicyclo[3.1.0]hexan-3- | azabicyclo[3.1.0]hexan- | 2.89 (s, 3 H), 3.10 (br dd, |
| | yl]-9H-pyrido[2,3-b]indol- | 6-yl]carbamate | J = 8.41, 4.39 Hz, 2 H), 3.51 |
| | 3-yl]-1-methyl-4-oxo-1,8- | BORONIC REAGENT: | (br d, J = 8.28 Hz, 2 H), 4.17 |
| | naphthyridine-3-carboxylic | Intermediate B1 | (s, 3 H), 6.60 (dd, J = 13.36, |
| | acid | CATALYST: Pd- | 6.21 Hz, 1 H), 8.16 (br d, |
| | 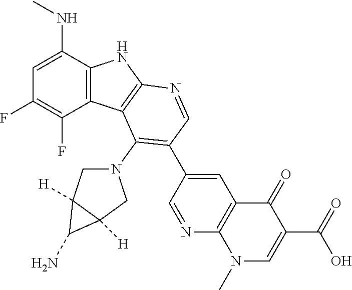 [SMILES: CNc1cc(F)c(F)c2c1[nH]c1ncc(-c3cnc4c(c3)c(=O)c(C(=O)O)cn4C)c(N3C[C@@H]4[C@@H](N)[C@@H]4C3)c12] | Ad₂nBuP Biphenyl Precat (CAS#: 1375477- 29-4) SOLVENT: THF/H₂O | J = 3.01 Hz, 2 H), 8.42 (s, 1 H), 8.62 (d, J = 1.63 Hz, 1 H), 9.02 (d, J = 1.63 Hz, 1 H), 9.24 (s, 1 H), 11.98 (br s, 1 H). MS: 532.1 ([M + H]+). |
| 1.09 | 6-[4-[2- | CORE: Intermediate A2 | ¹H NMR (400 MHz, CHCl₃- |
| | (Aminomethyl)pyrrolidin- | AMINE: tert-Butyl N- | d1) δ ppm: 9.02-9.13 (m, |
| | 1-yl]-5,6-difluoro-8- | (pyrrolidin-2- | 2H), 8.73 (m, 1H), 6.60 (m, |
| | (methylamino)-9H- | ylmethyl)carbamate | 1 H), 5.75-5.87 (m, 1H), |
| | pyrido[2,3-b]indol-3-yl]-1- | BORONIC REAGENT: | 4.11 (s, 3H), 3.62-3.65 (m, |
| | methyl-4-oxo-1,8- | Intermediate B1 | 5H), 3.10-3.20 (m, 1H), |
| | naphthyridine-3-carboxylic | CATALYST: | 2.85-2.92 (m, 5H), 1.70- |
| | acid | cataCXium A-Pd-G2 | 1.85 (m, 2H). |
| | 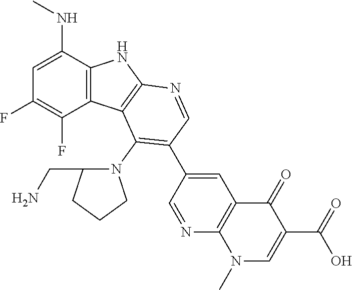 [SMILES: CNc1cc(F)c(F)c2c1[nH]c1ncc(-c3cnc4c(c3)c(=O)c(C(=O)O)cn4C)c(N3CCCC3CN)c12] | (Sigma-Aldrich, Catalog #: 761311) SOLVENT: Dioxane/H₂O | MS (ESI): 534.1 ([M + H]⁺). |
| 1.10 | 6-[4-[(2S)-2- | CORE: Intermediate A2 | ¹H NMR (400 MHz, |
| | (Aminomethyl)pyrrolidin- | AMINE: tert-Butyl N- | DMSO-d6) δ ppm: 9.24 (s, |
| | 1-yl]-5,6-difluoro-8- | [[(2S)-pyrrolidin-2- | 1H), 9.09 (d, J = 2.4 Hz, 1H), |
| | (methylamino)-9H- | yl]methyl]carbamate | 8.68 (d, J = 2.4 Hz, 1H), 8.28 |
| | pyrido[2,3-b]indol-3-yl]-1- | BORONIC REAGENT: | (s, 1H), 6.60 (m, 1H), 5.69 |
| | methyl-4-oxo-1,8- | Intermediate B1 | (m, 1H), 4.17 (s, 3H), 3.65 |
| | naphthyridine-3-carboxylic | CATALYST: | (m, 1H), 3.20 (m, 1H), 3.07 |
| | acid | cataCXium A-Pd-G2 | (m, 1H), 2.90 (d, J = 4.8 Hz, |
| | 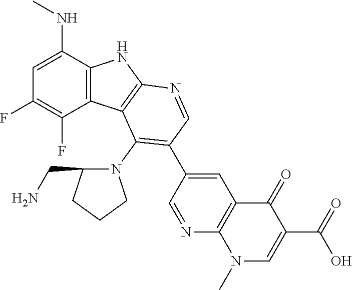 [SMILES: CNc1cc(F)c(F)c2c1[nH]c1ncc(-c3cnc4c(c3)c(=O)c(C(=O)O)cn4C)c(N3CCC[C@H]3CN)c12] | (Sigma-Aldrich, Catalog #: 761311) SOLVENT: Dioxane/H₂O | 3H), 2.55 (m, 2H), 2.32- 2.22 (m, 2H), 2.00-1.79 (m, 2H), 1.76-1.56 (m, 2H). MS (ESI): 534.3 ([M + H]⁺). |
| 1.11 | 6-[4-[3- | CORE: Intermediate A2 | ¹H NMR (400 MHz, |
| | (Aminomethyl)pyrrolidin- | AMINE: tert-Butyl N- | DMSO-d6) δ ppm 14.77 (br |
| | 1-yl]-5,6-difluoro-8- | (pyrrolidin-3- | s, 1 H) 11.84 (s, 1 H) 9.31 |
| | (methylamino)-9H- | ylmethyl)carbamate | (s, 1 H) 9.11 (d, J = 2.32 Hz, |
| | pyrido[2,3-b]indol-3-yl]-1- | BORONIC REAGENT: | 1 H) 8.68 (d, J = 2.32 Hz, 1 |
| | methyl-4-oxo-1,8- | Intermediate B1 | H) 8.31 (s, 1 H) 7.68 (br s, 3 |
| | naphthyridine-3-carboxylic | CATALYST: | H) 6.61 (dd, J = 13.51, 6.30 |
| | acid | cataCXium A-Pd-G2 | Hz, 1 H) 4.18 (s, 3 H) 3.26- |
| | 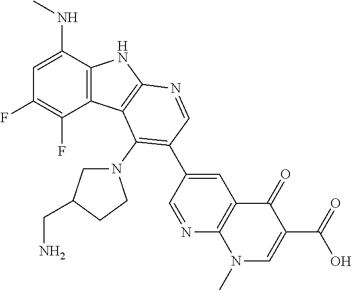 [SMILES: CNc1cc(F)c(F)c2c1[nH]c1ncc(-c3cnc4c(c3)c(=O)c(C(=O)O)cn4C)c(N3CCC(CN)C3)c12] | (Sigma-Aldrich, Catalog #: 761311) SOLVENT: Dioxane/H₂O | 3.36 (m, 2 H) 3.20 (br t, J = 6.48 Hz, 2 H) 2.88-2.98 (m, 3 H) 2.76-2.87 (m, 3 H) 2.02 (br s, 1 H) 1.47- 1.65 (m, 1 H) MS (ESI): 534.3 ([M + H]⁺). |
| 1.12 | trans-6-(5,6-Difluoro-8- | CORE: Intermediate A2 | ¹H NMR (400 MHz, |
| | (methylamino)-4-[3a- | AMINE: trans-tert-Butyl | DMSO-d6) δ ppm 9.29 (s, 1 |
| | amino-1,3,4,5,6,6a- | N-(2,3,4,5,6,6a- | H) 9.11 (s, 1 H) 8.62-8.67 |
| | hexahydrocyclopenta[c]pyr- | hexahydro-1H- | (m, 1 H) 8.29 (s, 1 H) 8.20 |
| | rol-2-yl]-9H-pyrido[2,3- | cyclopenta[c]pyrrol-3a- | (s, 1 H) 6.60 (dd, J = 13.45, |
| | b]indol-3-yl)-1-methyl-4- | yl)carbamate | 6.24 Hz, 1 H) 5.76 (br d, |
| | oxo-1,8-naphthyridine-3- | BORONIC REAGENT: | J = 3.55 Hz, 1 H) 4.11-4.18 |
| | carboxylic acid | Intermediate B1 | (m, 3 H) 3.96-4.09 (m, 1 |
| | 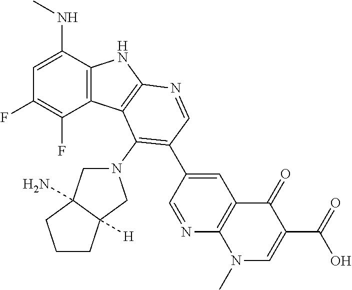 [SMILES: CNc1cc(F)c(F)c2c1[nH]c1ncc(-c3cnc4c(c3)c(=O)c(C(=O)O)cn4C)c(N3C[C@H]4CCC[C@@]4(N)C3)c12] | CATALYST: cataCXium A-Pd-G2 (Sigma-Aldrich, Catalog #: 761311) Dioxane/H₂O | H) 2.82-2.97 (m, 5 H) 2.70- 2.80 (m, 1 H) 1.70-1.85 (m, 1 H) 1.53-1.66 (m, 1 H) 1.27-1.48 (m, 5 H) MS (ESI): 560.8 ([M + H]⁺). |
| 1.13 | 6-[5-Cyano-6-fluoro-8- | CORE: Intermediate A4 | ¹H NMR (400 MHz, |
| | (methylamino)-4-[rel- | AMINE: tert-Butyl N- | DMSO-d6) δ ppm 9.30 (br |
| | (3aR,4R,6aS)-4-amino- | [rel-(3aR,4R,6aS)- | s, 1 H) 9.00-9.23 (m, 2 H) |
| | 3,3a,4,5,6,6a-hexahydro- | 1,2,3,3a,4,5,6,6a- | 8.66 (br s, 1 H) 8.21-8.39 |
| | 1H-cyclopenta[c]pyrrol-2- | octahydrocyclopenta[c] | (m, 1 H) 8.12 (s, 1 H) 6.90- |
| | yl]-9H-pyrido[2,3-b]indol- | pyrrol-4-yl]carbamate | 7.02 (m, 1 H) 6.61-6.70 |
| | 3-yl]-1-methyl-4-oxo-1,8- | BORONIC REAGENT: | (m, 1 H) 4.17 (s, 3 H) 3.40- |
| | naphthyridine-3-carboxylic | Intermediate B1 | 3.66 (m, 3 H) 3.37 (br d, |
| | acid | CATALYST: | J = 5.50 Hz, 1 H) 3.06-3.22 |
| | 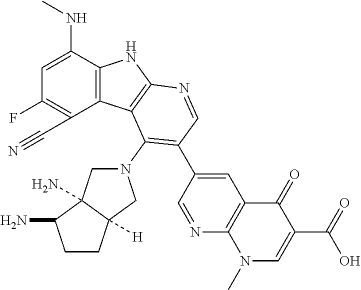 | cataCXium A-Pd-G2 (Sigma-Aldrich, Catalog #: 761311) SOLVENT: Dioxane/H₂O | (m, 2 H) 3.00 (d, J = 4.65 Hz, 4 H) 2.92 (br s, 1 H) 2.68- 2.80 (m, 1 H) 2.55-2.64 (m, 1 H) 1.60-1.72 (m, 1 H) 1.41-1.59 (m, 2 H) 1.06- 1.21 (m, 1 H) MS (ESI): 567.0 ([M + H]⁺). |
| 1.14 | 6-[4-[3-(Aminomethyl)-3- | CORE: Intermediate A2 | ¹H NMR (400 MHz, |
| | fluoro-pyrrolidin-1-yl]-5,6- | AMINE: tert-Butyl N- | MeOH-d4) δ 9.1-9.1 (m, |
| | difluoro-8-(methylamino)- | [(3-fluoropyrrolidin-3- | 1H), 8.9-9.0 (m, 1H), 8.7- |
| | 9H-pyrido[2,3-b]indol-3- | yl)methyl]carbamate | 8.8 (m, 1H), 8.2-8.3 (m, |
| | yl]-1-ethyl-4-oxo-1,8- | BORONIC REAGENT: | 1H), 8.0-8.0 (m, 1H), 6.5- |
| | naphthyridine-3-carboxylic | Intermediate B2 | 6.6 (m, 1H), 4.7-4.7 (m, |
| | acid | CATALYST: x-Phos | 2H), 3.5-3.5 (m, 1H), 3.3- |
| | 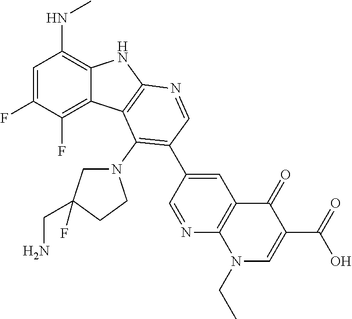 [SMILES: CCn1cc(C(=O)O)c(=O)c2cc(-c3cnc4[nH]c5c(NC)cc(F)c(F)c5c4c3N3CCC(F)(CN)C3)cnc21] | Pd G2(CAS: 1310584- 14-5) SOLVENT: THF/H₂O | 3.4 (m, 2H), 3.3-3.3 (m, 2H), 2.9-2.9 (m, 3H), 2.1- 2.2 (m, 2H), 1.89 (s, 1H), 1.49 (t, 3H, J = 7.0 Hz) MS (ESI): 566.1([M + H]⁺). |
| 1.15 | 6-[4-[3-(Aminomethyl)-3- | CORE: Intermediate A2 | ¹H NMR (400 MHz, |
| | fluoro-1-piperidyl]-5,6- | AMINE: tert-Butyl N- | MeOH-d4) δ 8.9-9.1 (m, |
| | difluoro-8-(methylamino)- | [(3-fluoro-3- | 2H), 8.7-8.8 (m, 1H), 8.4- |
| | 9H-pyrido[2,3-b]indol-3- | piperidyl)methyl]carbamate | 8.5 (m, 2H), 8.2-8.3 (m, |
| | yl]-1-ethyl-4-oxo-1,8- | BORONIC REAGENT: | 1H), 6.5-6.6 (m, 1H), 4.6- |
| | naphthyridine-3-carboxylic | Intermediate B2 | 4.7 (m, 1H), 4.4-4.5 (m, |
| | acid | CATALYST: x-Phos Pd | 2H), 3.3-3.4 (m, 3H), 2.9- |
| | 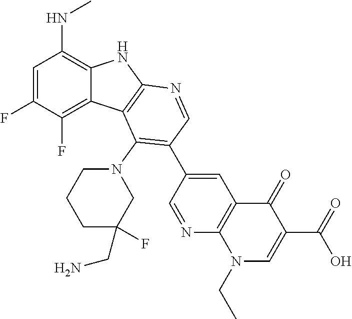 [SMILES: CCn1cc(C(=O)O)c(=O)c2cc(-c3cnc4[nH]c5c(NC)cc(F)c(F)c5c4c3N3CCCC(F)(CN)C3)cnc21] | G2(CAS: 1310584-14-5) SOLVENT: THF/H₂O | 3.1 (m, 3H), 2.9-2.9 (m, 5H), 1.8-1.9 (m, 1H), 1.5- 1.5 (m, 3H) MS (ESI): 580.6 ([M + H]⁺). |
| 1.16 | 6-[4-[3-(Aminomethyl)-3- | CORE: Intermediate A2 | ¹H NMR (400 MHz, |
| | fluoro-pyrrolidin-1-yl]-5,6- | AMINE: tert-Butyl N- | DMSO-d6) δ 11.7-11.9 (m, |
| | difluoro-8-(methylamino)- | [(3-fluoropyrrolidin-3- | 1H), 9.2-9.3 (m, 1H), 9.0- |
| | 9H-pyrido[2,3-b]indol-3- | yl)methyl]carbamate | 9.1 (m, 1H), 8.6-8.7 (m, |
| | yl]-1-methyl-4-oxo-1,8- | BORONIC REAGENT: | 1H), 8.2-8.3 (m, 1H), 7.96 |
| | naphthyridine-3-carboxylic | Intermediate B1 | (br s, 3H), 6.4-6.7 (m, 1H), |
| | acid | CATALYST: x-phos Pd | 4.1-4.1 (m, 3H), 3.1-3.2 (m, |
| | 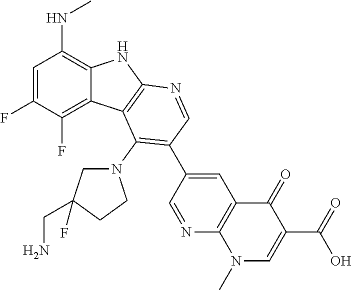 [SMILES: CNc1cc(F)c(F)c2c1[nH]c1ncc(-c3cnc4c(c3)c(=O)c(C(=O)O)cn4C)c(N3CCC(F)(CN)C3)c12] | G2(CAS: 1310584-14-5) SOLVENT: THF/H₂O | 6H), 2.8-2.9 (m, 3H), 2.0- 2.1 (m, 2H) MS (ESI): 552.2 ([M + H]⁺). |
| 1.17 | 6-[4-[(3R)-3- | CORE: Intermediate A2 | ¹H NMR (400 MHz, |
| | Aminopyrrolidin-1-yl]-5,6- | AMINE: tert-Butyl N- | DMSO-d6) δ 11.7-11.9 (m, |
| | difluoro-8-(methylamino)- | [(3R)-pyrrolidin-3- | 1H), 9.2-9.3 (m, 1H), 9.0- |
| | 9H-pyrido[2,3-b]indol-3- | yl]carbamate | 9.1 (m, 1H), 8.6-8.7 (m, |
| | yl]-1-methyl-4-oxo-1,8- | BORONIC REAGENT: | 1H), 8.1-8.2 (m, 1H), 7.8- |
| | naphthyridine-3-carboxylic | Intermediate B1 | 7.9 (m, 3H), 6.5-6.6 (m, |
| | acid | CATALYST: x-Phos Pd | 1H), 4.0-4.2 (m, 3H), 3.5- |
| | 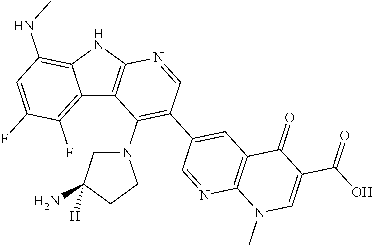 [SMILES: CNc1cc(F)c(F)c2c1[nH]c1ncc(-c3cnc4c(c3)c(=O)c(C(=O)O)cn4C)c(N3CC[C@@H](N)C3)c12] | G2(CAS: 1310584-14-5) SOLVENT: THF/H₂O | 3.6 (m, 1H), 3.2-3.3 (m, 1H), 2.7-2.9 (m, 5H), 2.1- 2.2 (m, 1H), 1.9-2.0 (m, 1H), 1.7-1.8 (m, 1H) MS (ESI): 520.4 ([M + H]⁺). |

### Example 2.01

### Trans-6-[4-(2-amino-5-azaspiro[2.4]heptan-5-yl)-5,6-difluoro-8-(methylamino)-9H-pyrido[2,3-b]indol-3-yl]-1-methyl-4-oxo-1,8-naphthyridine-3-carboxylic acid

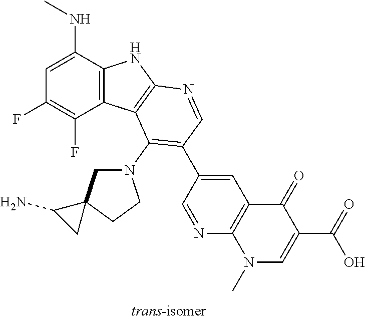

### Step (a) Preparation of tert-butyl N-[4-[2-(tert-butoxycarbonylamino)-5-azaspiro[2.4]heptan-5-yl]-3-chloro-5,6-difluoro-9H-pyrido[2,3-b]indol-8-yl]-N-methyl-carbamate

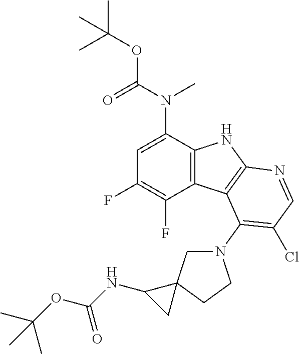 [SMILES: CN(C(=O)OC(C)(C)C)c1cc(F)c(F)c2c1[nH]c1ncc(Cl)c(N3CCC4(CC4NC(=O)OC(C)(C)C)C3)c12]

A solution of mixture of tert-butyl N-(3,4-dichloro-5,6-difluoro-9H-pyrido[2,3-b]indol-8-yl)-N-methyl-carbamate (1 g, 2.49 mmol, Intermediate A2), tert-butyl N-(5-azaspiro[2.4]heptan-2-yl)carbamate (634 mg, 2.98 mmol), and Et₃N (1.04 mL, 7.46 mmol) in DMSO (5 mL) was stirred at 120° C. for 16 h. After the mixture was cooled back to r.t., EtOAc (100 mL) was added and the mixture was washed with brine (30 mL) twice. The organic layer was dried over anhy. Na₂SO₄, filtered, and concentrated in vacuo to give a crude product, which was purified by silica gel flash chromatography (2-20% EtOAc in petroleum ether) to give tert-butyl N-[4-[2-(tert-butoxycarbonylamino)-5-azaspiro[2.4]heptan-5-yl]-3-chloro-5,6-difluoro-9H-pyrido[2,3-b]indol-8-yl]-N-methyl-carbamate (850 mg, 59.1% yield) as a yellow solid. MS (ESI): 580.3 ([{³⁵Cl}M+H]⁺), 578.3 ([{³⁷Cl}M+H]⁺).

### Step (b) Preparation of ethyl 6-[4-[2-(tert-butoxycarbonylamino)-5-azaspiro[2.4]heptan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylate

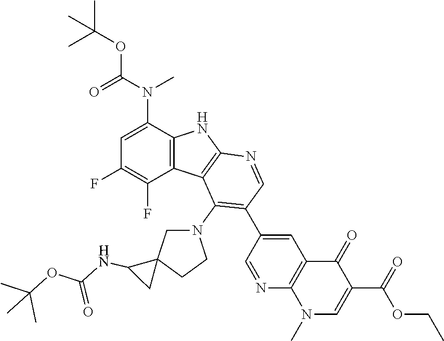 [SMILES: CCOC(=O)c1cn(C)c2ncc(-c3cnc4[nH]c5c(N(C)C(=O)OC(C)(C)C)cc(F)c(F)c5c4c3N3CCC4(CC4NC(=O)OC(C)(C)C)C3)cc2c1=O]

A solution of mixture of tert-butyl N-[4-[2-(tert-butoxycarbonylamino)-5-azaspiro[2.4]heptan-5-yl]-3-chloro-5,6-difluoro-9H-pyrido[2,3-b]indol-8-yl]-N-methyl-carbamate (400 mg, 0.690 mmol), ethyl 1-methyl-4-oxo-6-(4,4,5,5-tetramethyl-1,3,2-dioxaborolan-2-yl)-1,4-dihydro-1,8-naphthyridine-3-carboxylate (286 mg, 1.04 mmol, Intermediate B1), Pd-Ad₂nBuP Biphenyl Precat (46 mg, 0.070 mmol), and K₃PO₄ (440 mg, 2.08 mmol) in TRF (10 mL) and water (1 mL) was stirred at 70° C. for 16 h under Argon. After the reaction mixture was cooled back to r.t., EtOAc (50 mL) was added and the mixture was washed with brine (30 mL) twice. The organic layer was dried over anhy. Na₂SO₄, filtered, and concentrated in vacuo to give a crude product, which was purified by silica gel flash chromatography (20% EtOAc in petroleum ether, then 10% MeOH in DCM) to give ethyl 6-[4-[2-(tert-butoxycarbonylamino)-5-azaspiro[2.4]heptan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylate (300 mg, 56% yield) as a yellow solid. MS (ESI): 774.6 ([M+H]⁺).

### Step (c) Preparation of trans-ethyl 6-[4-[2-(tert-butoxycarbonylamino)-5-azaspiro[2.4]heptan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylate and cis-ethyl 6-[4-[2-(tert-butoxycarbonylamino)-5-azaspiro[2.4]heptan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylate

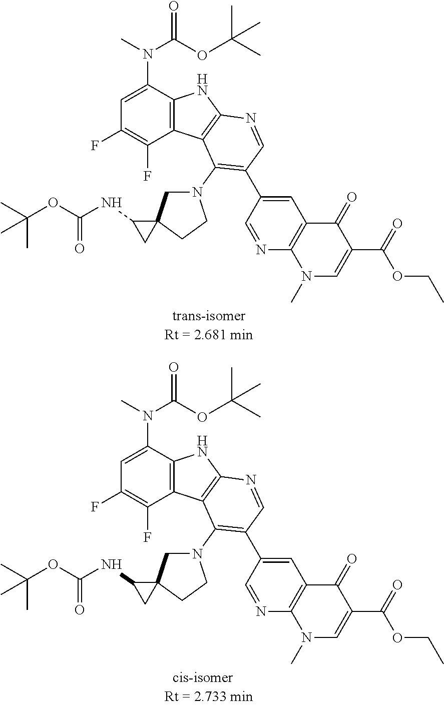

260 mg of ethyl 6-[4-[2-(tert-butoxycarbonylamino)-5-azaspiro[2.4]heptan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylate was subject to prep-HPLC separation (Column: Phenomenex Synergi C18 150×30 mm×4 um, Mobile phase: water (0.225% FA)-ACN, from 38% ACN in water to 68% ACN in water) to give trans-ethyl 6-[4-[2-(tert-butoxycarbonylamino)-5-azaspiro[2.4]heptan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylate (100 mg, 38.5% yield, HPLC retention time Rt=2.681 min) as a yellow solid (MS (ESI): 774.6 [M+H]⁺) and cis ethyl 6-[4-[2-(tert-butoxycarbonylamino)-5-azaspiro[2.4]heptan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylate (60 mg, 23.1% yield, HPLC retention time Rt=2.733 min) as another yellow solid (MS (ESI): 774.6 [M+H]⁺).

### Step (d) Preparation of trans-6-[8-[tert-butoxycarbonyl(methyl)amino]-5,6-difluoro-4-[2-(tert-butoxycarbonylamino)-5-azaspiro[2.4]heptan-5-yl]-9H-pyrido[2,3-b]indol-3-yl]-1-methyl-4-oxo-1,8-naphthyridine-3-carboxylic acid

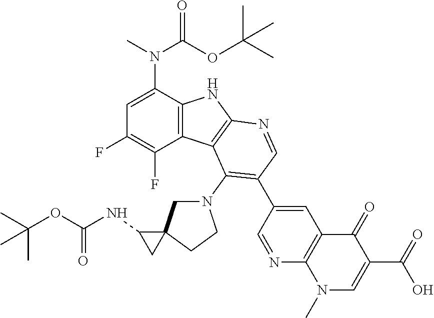

To a mixture of trans-ethyl 6-[4-[2-(tert-butoxycarbonylamino)-5-azaspiro[2.4]heptan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylate (100 mg, 0.150 mmol) in EtOH (5 mL) was added aq. NaOH solution (1 mL, 1 mmol), and the mixture was stirred at 15° C. for 1 h. Afterwards, the mixture solution was concentrated in vacuo to give a residue, which was re-suspended in H₂O (40 mL) and extracted with EtOAc (20 mL) three times. The separated aqueous layer was acidified with aq. HCl solution (1N) to pH=5-6, and extracted with DCM (30 mL) three times. Combined organics were then washed with brine (30 mL) three times, dried over anhy. Na₂SO₄, filtered, and concentrated in vacuo to give trans-6-[8-[tert-butoxycarbonyl(methyl)amino]-5,6-difluoro-4-[2-(tert-butoxycarbonylamino)-5-azaspiro[2.4]heptan-5-yl]-9H-pyrido[2,3-b]indol-3-yl]-1-methyl-4-oxo-1,8-naphthyridine-3-carboxylic acid (90 mg, 93.8% yield) as a yellow solid. MS (ESI): 746.2 ([M+H]⁺).

### Step (e) Preparation of trans-6-[4-(2-Amino-5-azaspiro[2.4]heptan-5-yl)-5,6-difluoro-8-(methylamino)-9H-pyrido[2,3-b]indol-3-yl]-1-methyl-4-oxo-1,8-naphthyridine-3-carboxylic acid

To trans-6-[4-[2-(tert-butoxycarbonylamino)-5-azaspiro[2.4]heptan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylic acid (90 mg, 0.120 mmol) in DCM (5 mL) was added TFA (1 mL, 12.98 mmol), and the reaction mixture was stirred at 15° C. for 0.5 h. Afterwards, the mixture was concentrated in vacuo to give a crude product, which was purified by prep-HPLC (0.1% TFA as additive) to give trans-6-[4-(2-amino-5-azaspiro[2.4]heptan-5-yl)-5,6-difluoro-8-(methylamino)-9H-pyrido[2,3-b]indol-3-yl]-1-methyl-4-oxo-1,8-naphthyridine-3-carboxylic acid (41.5 mg, 63.5% yield) as a yellow solid. MS (ESI): 546.2 ([M+H]⁺). ¹H NMR (400 MHz, CHCl₃-d1) δppm: 11.90 (s, 1H), 9.31 (s, 1H), 9.14 (d, 1H), 8.72 (d, 1H), 8.30 (s, 1H), 8.00 (m 3H), 6.62 (m, 1H), 4.19 (s, 3H), 3.33 (m, 2H), 3.24 (m, 2H), 2.91 (s, 3H), 2.71-2.63 (m, 1H), 2.07-1.94 (m, 1H), 1.68 (m, 1H), 0.98 (m, 1H), 0.70 (m, 1H).

### Example 2.02

### Cis-6-[4-(2-amino-5-azaspiro[2.4]heptan-5-yl)-5,6-difluoro-8-(methylamino)-9H-pyrido[2,3-b]indol-3-yl]-1-methyl-4-oxo-1,8-naphthyridine-3-carboxylic acid

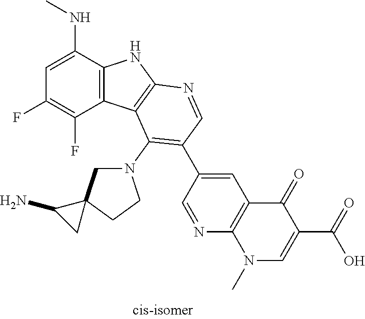

The title compound was prepared in analogy to the synthesis of Example 2.01, replacing trans-ethyl 6-[4-[2-(tert-butoxycarbonylamino)-5-azaspiro[2.4]heptan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylate with cis-ethyl 6-[4-[2-(tert-butoxycarbonylamino)-5-azaspiro[2.4]heptan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylate in step (d). MS (ESI): 546.3 ([M+H]⁺). ¹H NMR (400 MHz, CHCl₃-d1) δppm: 11.91 (s, 1H), 9.29 (s, 1H), 9.15-9.07 (d, 1H), 8.72-8.67 (d, 1H), 8.29 (s, 1H), 8.18 (m, 2H), 6.61 (m, 1H), 4.18 (s, 3H), 3.40 (m, 2H), 3.19 (m, 1H), 3.00 (m, 1H), 2.90 (s, 3H), 2.79 (m, 1H), 2.02 (m, 1H), 1.97-1.86 (m, 1H), 0.89-0.79 (m, 2H).

### Example 3.01

### 6-[4-[(2R,3R)-2-Amino-5-azaspiro[2.4]heptan-5-yl]-5,6-difluoro-8-(methylamino)-9H-pyrido[2,3-b]indol-3-yl]-1-methyl-4-oxo-1,8-naphthyridine-3-carboxylic acid

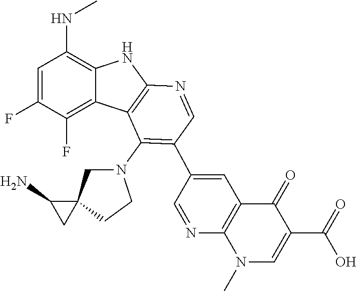 [SMILES: CNc1cc(F)c(F)c2c1[nH]c1ncc(-c3cnc4c(c3)c(=O)c(C(=O)O)cn4C)c(N3CC[C@@]4(C[C@H]4N)C3)c12]

### Step (a) Preparation of tert-butyl N-[4-[2-(tert-butoxycarbonylamino)-5-azaspiro[2.4]heptan-5-yl]-3-chloro-5,6-difluoro-9H-pyrido[2,3-b]indol-8-yl]-N-methyl-carbamate

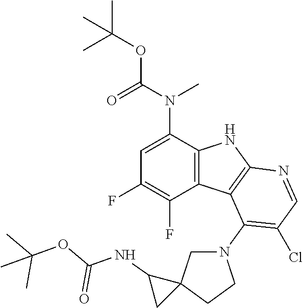 [SMILES: CN(C(=O)OC(C)(C)C)c1cc(F)c(F)c2c1[nH]c1ncc(Cl)c(N3CCC4(CC4NC(=O)OC(C)(C)C)C3)c12]

A solution of tert-butyl N-(3,4-dichloro-5,6-difluoro-9H-pyrido[2,3-b]indol-8-yl)-N-methyl carbamate (400 mg, 1 mmol, Intermediate A2), tert-butyl 5-azaspiro[2.4]heptan-1-ylcarbamate (390.8 mg, 1.8 mmol) and DIPEA (0.57 mL, 3.2 mmol) in DMSO (5 mL) was stirred at 110° C. for 12 h. After it was cooled back to r.t., the mixture was diluted with EtOAc (100 mL), poured into water (100 mL), and extracted with EtOAc (100 mL). The organic layer was washed with aq. NaCl solution (100 mL), dried over anhy. Na₂SO₄, filtered, and concentrated in vacuo to give a crude product, which was purified by prep-TLC (petroleum ether:EtOAc=2:1) to give tert-butyl N-[4-[2-(tert-butoxycarbonylamino)-5-azaspiro[2.4]heptan-5-yl]-3-chloro-5,6-difluoro-9H-pyrido[2,3-b]indol-8-yl]-N-methyl-carbamate (550 mg, 95.7% yield) as a yellow solid. MS (ESI): 770.6 ([{³⁵Cl}M+H]⁺).

### Step (b) Preparation of tert-butyl(4-((1S,3R)-1-((tert-butoxycarbonyl)amino)-5-azaspiro[2.4]heptan-5-yl)-3-chloro-5,6-difluoro-9H-pyrido[2,3-b]indol-8-yl)(methyl)carbamate

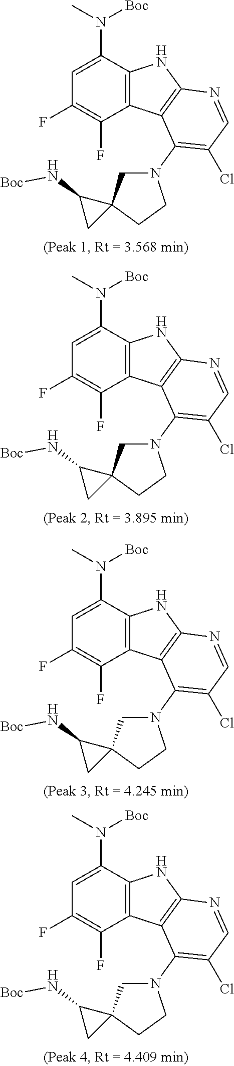

550 mg of tert-butyl N-[4-[2-(tert-butoxycarbonylamino)-5-azaspiro[2.4]heptan-5-yl]-3-chloro-5,6-difluoro-9H-pyrido[2,3-b]indol-8-yl]-N-methyl-carbamate_was subject to chiral SFC separation to give tert-butyl N-[4-[(2R,3R)-2-(tert-butoxycarbonylamino)-5-azaspiro[2.4]heptan-5-yl]-3-chloro-5,6-difluoro-9H-pyrido[2,3-b]indol-8-yl]-N-methyl-carbamate (110 mg, Peak 1, Rt=3.568 min), tert-butyl N-[4-[(2S,3R)-2-(tert-butoxycarbonylamino)-5-azaspiro[2.4]heptan-5-yl]-3-chloro-5,6-difluoro-9H-pyrido[2,3-b]indol-8-yl]-N-methyl-carbamate (100 mg, Peak 2, Rt=3.895 min), tert-butyl N-[4-[(2R,3S)-2-(tert-butoxycarbonylamino)-5-azaspiro[2.4]heptan-5-yl]-3-chloro-5,6-difluoro-9H-pyrido[2,3-b]indol-8-yl]-N-methyl-carbamate (100 mg, Peak 3, Rt=4.245 min), and tert-butyl N-[4-[(2S,3S)-2-(tert-butoxycarbonylamino)-5-azaspiro[2.4]heptan-5-yl]-3-chloro-5,6-difluoro-9H-pyrido[2,3-b]indol-8-yl]-N-methyl-carbamate (120 mg, Peak 4, Rt=4.409 min), MS (ESI): 770.6 ([{³⁵Cl}M+H]⁺).

SFC condition: Column: AD, 250×20 mm I.D., 5 μm. Mobile phase: A for CO₂ and B for IPA (0.1% NH₃H₂O); Gradient: B 20%. Flow rate: 50 mL/min. Back pressure: 100 bar. Column temperature: 35° C.

### Step (c) Preparation of ethyl 6-[4-[(2R,3R)-2-(tert-butoxycarbonylamino)-5-azaspiro[2.4]heptan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylate

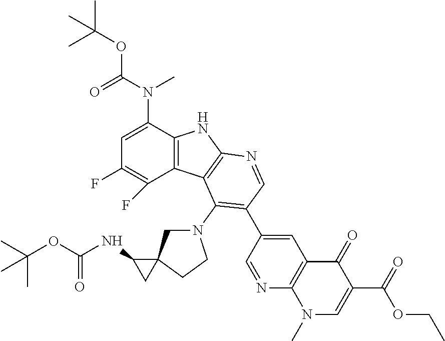 [SMILES: CCOC(=O)c1cn(C)c2ncc(-c3cnc4[nH]c5c(N(C)C(=O)OC(C)(C)C)cc(F)c(F)c5c4c3N3CC[C@@]4(C[C@H]4NC(=O)OC(C)(C)C)C3)cc2c1=O]

To a solution of tert-butyl N-[4-[(2S,3R)-2-(tert-butoxycarbonylamino)-5-azaspiro[2.4]heptan-5-yl]-3-chloro-5,6-difluoro-9H-pyrido[2,3-b]indol-8-yl]-N-methyl-carbamate (0.050 g, 86.5 μmol) in THF (1 mL) and water (0.01 mL) were added ethyl 1-methyl-4-oxo-6-(4,4,5,5-tetramethyl-1,3,2-dioxaborolan-2-yl)-1,8-naphthyridine-3-carboxylate (93 mg, 0.34 mmol) (Intermediate B1, 55.8 mg, 156 μmol), Pd-Ad₂nBuP Biphenyl Precat (20.4 mg, 0.026 mmol), and K₃PO₄ (55.1 mg, 0.310 mmol) in one portion in a glove box under Argon atmosphere. The mixture solution was then stirred at 70° C. for 16 h under Ar. After cooled back to the r.t., the reaction mixture was diluted with water (10 mL) and extracted with EtOAc (10 mL) three times. Combined organics were washed with brine (20 mL), dried with anhy. Na₂SO₄, filtered, and concentrated in vacuo to give a crude product, which was purified by prep-TLC (DCM/MeOH=10/1) to give ethyl 6-[4-[(2R,3R)-2-(tert-butoxycarbonylamino)-5-azaspiro[2.4]heptan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylate (42 mg, 0.054 mmol, 62.7% yield). MS (ESI): 774.7 [{³⁵Cl} (M+H)⁺].

### Step (d) Preparation of 6-[4-[(2R,3R)-2-(tert-butoxycarbonylamino)-5-azaspiro[2.4]heptan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylic acid

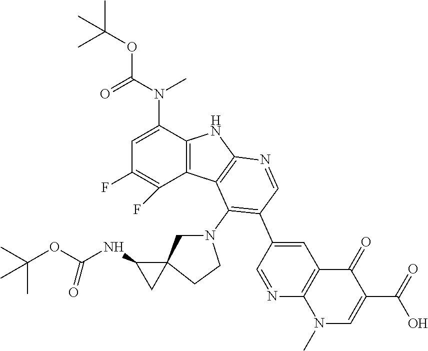 [SMILES: CN(C(=O)OC(C)(C)C)c1cc(F)c(F)c2c1[nH]c1ncc(-c3cnc4c(c3)c(=O)c(C(=O)O)cn4C)c(N3CC[C@@]4(C[C@H]4NC(=O)OC(C)(C)C)C3)c12]

To a solution of ethyl 6-[4-[(2R,3R)-2-(tert-butoxycarbonylamino)-5-azaspiro[2.4]heptan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylate (42 mg, 0.054 mmol) in THF (1.5 mL) and water (1.5 mL) was added NaOH (32.6 mg, 0.780 mmol) in one portion, and the resulting solution was stirred at 20° C. for 1 h. The mixture was concentrated in vacuo to give a residue, which was acidified with 1 N HCl to pH=5-6, and extracted with EtOAc (30 mL) two times. The aqueous phase was extracted with additional EtOAc (10 mL) three times. Combined organics were dried over anhy. Na₂SO₄, filtered, and concentrated in vacuo to give 6-[4-[(2R,3R)-2-(tert-butoxycarbonylamino)-5-azaspiro[2.4]heptan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylic acid (40 mg, 100% yield) as a white solid. MS (ESI): 746.4 [{³⁵Cl} (M+H)⁺].

### Step (e) Preparation of 6-[4-[(2R,3R)-2-amino-5-azaspiro[2.4]heptan-5-yl]-5,6-difluoro-8-(methylamino)-9H-pyrido[2,3-b]indol-3-yl]-1-methyl-4-oxo-1,8-naphthyridine-3-carboxylic acid

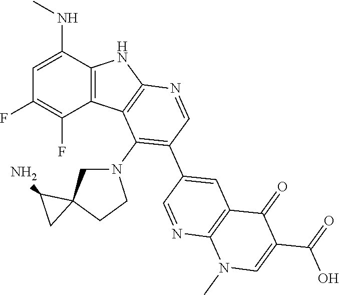 [SMILES: CNc1cc(F)c(F)c2c1[nH]c1ncc(-c3cnc4c(c3)c(=O)c(C(=O)O)cn4C)c(N3CC[C@@]4(C[C@H]4N)C3)c12]

To a solution of 6-[4-[(2R,3R)-2-(tert-butoxycarbonylamino)-5-azaspiro[2.4]heptan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylic acid (40 mg, 0.054 mmol) in DCM (3 mL) was added TFA (2 mL), and the solution was stirred at 20° C. for 2 h. The reaction mixture was concentrated in vacuo to give a crude product, which was purified by prep-HPLC (water (1% TFA)-ACN) to give 6-[4-[(2R,3R)-2-amino-5-azaspiro[2.4]heptan-5-yl]-5,6-difluoro-8-(methylamino)-9H-pyrido[2,3-b]indol-3-yl]-1-methyl-4-oxo-1,8-naphthyridine-3-carboxylic acid as a white solid (3.9 mg, 12.3% yield). MS (ESI): 546.1 [{³⁵Cl}(M+H)⁺]. ¹H NMR (DMSO-d₆, 400 MHz) δ 9.2-9.2 (m, 1H), 9.0-9.1 (m, 1H), 8.6-8.7 (m, 1H), 8.24 (br d, 2H, J=2.6 Hz), 6.5-6.6 (m, 2H), 5.6-5.6 (m, 1H), 4.1-4.1 (m, 3H), 3.1-3.1 (m, 4H), 2.9-3.0 (m, 2H), 2.8-2.9 (m, 3H), 2.6-2.6 (m, 1H), 2.26 (m, 1H, J=1.8, 3.5 Hz), 1.66 (m, 1H), 0.99 (m, 1H).

### Example 3.02

### 6-[4-[(2S,3R)-2-Amino-5-azaspiro[2.4]heptan-5-yl]-5,6-difluoro-8-(methylamino)-9H-pyrido[2,3-b]indol-3-yl]-1-methyl-4-oxo-1,8-naphthyridine-3-carboxylic acid

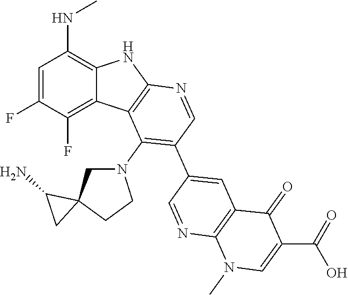 [SMILES: CNc1cc(F)c(F)c2c1[nH]c1ncc(-c3cnc4c(c3)c(=O)c(C(=O)O)cn4C)c(N3CC[C@@]4(C[C@@H]4N)C3)c12]

The title compound was prepared in analogy to Example 3.01 by replacing tert-butyl N-[4-[(2R,3R)-2-(tert-butoxycarbonylamino)-5-azaspiro[2.4]heptan-5-yl]-3-chloro-5,6-difluoro-9H-pyrido[2,3-b]indol-8-yl]-N-methyl-carbamate with tert-butyl N-[4-[(2S,3R)-2-(tert-butoxycarbonylamino)-5-azaspiro[2.4]heptan-5-yl]-3-chloro-5,6-difluoro-9H-pyrido[2,3-b]indol-8-yl]-N-methyl-carbamate in step (b). MS (ESI): 546.2 [{³⁵Cl}(M+H)⁺]. ¹H NMR (DMSO-d₆, 400 MHz) δ 9.2-9.2 (m, 1H), 9.0-9.1 (m, 1H), 8.6-8.7 (m, 1H), 8.1-8.3 (m, 1H), 6.8-7.0 (m, 1H), 6.4-6.6 (m, 1H), 5.5-5.7 (m, 1H), 4.0-4.1 (m, 3H), 3.12 (br s, 2H), 3.02 (br d, 4H, J=9.5 Hz), 2.9-2.9 (m, 1H), 2.8-2.8 (m, 1H), 2.83 (br d, 3H, J=4.8 Hz), 2.52 (m, 3H), 2.12 (br dd, 2H, J=4.1, 7.2 Hz), 1.64 (m, 1H), 0.97 (m, 1H).

### Example 3.03

### 6-[4-[(2R,3S)-2-Amino-5-azaspiro[2.4]heptan-5-yl]-5,6-difluoro-8-(methylamino)-9H-pyrido[2,3-b]indol-3-yl]-1-methyl-4-oxo-1,8-naphthyridine-3-carboxylic acid

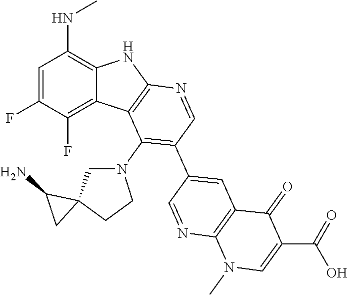 [SMILES: CNc1cc(F)c(F)c2c1[nH]c1ncc(-c3cnc4c(c3)c(=O)c(C(=O)O)cn4C)c(N3CC[C@]4(C[C@H]4N)C3)c12]

The title compound was prepared in analogy to Example 3.01 by replacing tert-butyl N-[4-[(2R,3R)-2-(tert-butoxycarbonylamino)-5-azaspiro[2.4]heptan-5-yl]-3-chloro-5,6-difluoro-9H-pyrido[2,3-b]indol-8-yl]-N-methyl-carbamate with tert-butyl N-[4-[(2R,3S)-2-(tert-butoxycarbonylamino)-5-azaspiro[2.4]heptan-5-yl]-3-chloro-5,6-difluoro-9H-pyrido[2,3-b]indol-8-yl]-N-methyl-carbamate in step (b). MS (ESI): 546.1 [{³⁵Cl}(M+H)⁺]. ¹H NMR (DMSO-d₆, 400 MHz) δ 9.2-9.3 (m, 1H), 9.0-9.1 (m, 1H), 8.6-8.6 (m, 1H), 8.1-8.2 (m, 1H), 6.52 (dd, 2H, J=6.2, 13.8 Hz), 5.6-5.7 (m, 1H), 4.1-4.1 (m, 3H), 3.1-3.1 (m, 4H), 3.0-3.0 (m, 2H), 2.83 (br d, 5H, J=4.9 Hz), 2.1-2.2 (m, 1H), 1.6-1.7 (m, 2H), 0.83 (m, 2H).

### Example 3.04

### 6-[4-[(2S,3S)-2-Amino-5-azaspiro[2.4]heptan-5-yl]-5,6-difluoro-8-(methylamino)-9H-pyrido[2,3-b]indol-3-yl]-1-methyl-4-oxo-1,8-naphthyridine-3-carboxylic acid

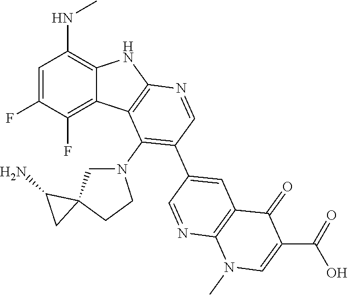 [SMILES: CNc1cc(F)c(F)c2c1[nH]c1ncc(-c3cnc4c(c3)c(=O)c(C(=O)O)cn4C)c(N3CC[C@]4(C[C@@H]4N)C3)c12]

The title compound was prepared in analogy to Example 3.01 by replacing tert-butyl N-[4-[(2R,3R)-2-(tert-butoxycarbonylamino)-5-azaspiro[2.4]heptan-5-yl]-3-chloro-5,6-difluoro-9H-pyrido[2,3-b]indol-8-yl]-N-methyl-carbamate with tert-butyl N-[4-[(2S,3S)-2-(tert-butoxycarbonylamino)-5-azaspiro[2.4]heptan-5-yl]-3-chloro-5,6-difluoro-9H-pyrido[2,3-b]indol-8-yl]-N-methyl-carbamate in step (b). MS (ESI): 546.1 [{³⁵Cl}(M+H)⁺]. (DMSO-d₆, 400 MHz) δ 9.0-9.0 (m, 1H), 8.8-8.9 (m, 1H), 8.4-8.5 (m, 1H), 8.0-8.0 (m, 1H), 6.3-6.4 (m, 1H), 5.4-5.5 (m, 1H), 3.93 (s, 3H), 3.0-3.1 (m, 3H), 2.8-2.8 (m, 1H), 2.6-2.7 (m, 3H), 2.5-2.5 (m, 1H), 1.8-1.9 (m, 1H), 1.6-1.7 (m, 1H), 1.5-1.6 (m, 1H), 0.83 (m, 2H).

### Example 4.01

### 6-[4-[(2R,3R)-2-Amino-5-azaspiro[2.4]heptan-5-yl]-5-chloro-6-fluoro-8-(methylamino)-9H-pyrido[2,3-b]indol-3-yl]-1-methyl-4-oxo-1,8-naphthyridine-3-carboxylic acid

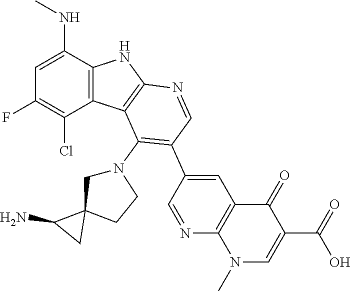 [SMILES: CNc1cc(F)c(Cl)c2c1[nH]c1ncc(-c3cnc4c(c3)c(=O)c(C(=O)O)cn4C)c(N3CC[C@@]4(C[C@H]4N)C3)c12]

### Step (a) Preparation of cis-tert-butyl N-[3-bromo-4-[2-(tert-butoxycarbonylamino)-5-azaspiro[2.4]heptan-5-yl]-5-chloro-6-fluoro-9H-pyrido[2,3-b]indol-8-yl]-N-methyl-carbamate and trans-tert-butyl N-[3-bromo-4-[2-(tert-butoxycarbonylamino)-5-azaspiro[2.4]heptan-5-yl]-5-chloro-6-fluoro-9H-pyrido[2,3-b]indol-8-yl]-N-methyl-carbamate

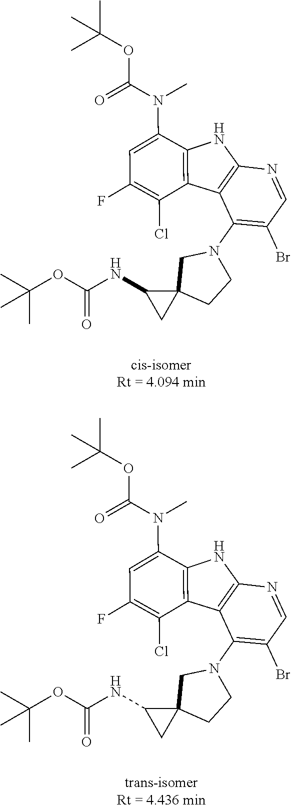

To a solution of tert-butyl (3-bromo-4,5-dichloro-6-fluoro-9H-pyrido[2,3-b]indol-8-yl) (methyl)carbamate (900 mg, 1.94 mmol, Intermediate A5), tert-butyl 5-azaspiro[2.4]heptan-1-ylcarbamate (500 mg, 2.36 mmol) in sulfolane (10 mL) was added DIPEA (750 mg, 5.80 mmol), and the solution was stirred at 100° C. for 2 h. After cooled back to r.t., the mixture was poured into water (100 mL), and extracted with EtOAc (80 mL) two times. Combined organics were dried over anhy. Na₂SO₄, filtered, and concentrated in vacuo to give a crude product, which was purified by prep-HPLC (H₂O (0.225% TFA)-ACN) to give cis-tert-butyl N-[3-bromo-4-[2-(tert-butoxycarbonylamino)-5-azaspiro[2.4]heptan-5-yl]-5-chloro-6-fluoro-9H-pyrido[2,3-b]indol-8-yl]-N-methyl-carbamate (peak 1, Rt=4.094 min, 710 mg, 57.2% yield) as a yellow solid and trans-tert-butyl N-[3-bromo-4-[2-(tert-butoxycarbonylamino)-5-azaspiro[2.4]heptan-5-yl]-5-chloro-6-fluoro-9H-pyrido[2,3-b]indol-8-yl]-N-methyl-carbamate (peak 2, Rt=4.346 min, 320 mg, 25.8% yield) as another yellow solid. MS (ESI): 640.0 [{³⁵Cl+⁸¹Br & ³⁷Cl+⁷⁹Br} (M+H)⁺] (peak 1); MS (ESI): 639.9 [{³⁵Cl+⁸¹Br & ³⁷Cl+⁷⁹Br}(M+H)⁺] (peak 2).

### Step (b) Preparation of tert-butyl(3-bromo-4-((1S,3R)-1-((tert-butoxycarbonyl)amino)-5-azaspiro[2.4]heptan-5-yl)-5-chloro-6-fluoro-9H-pyrido[2,3-b]indol-8-yl) (methyl)carbamate and tert-butyl(3-bromo-4-((1R,3S)-1-((tert-butoxycarbonyl)amino)-5-azaspiro[2.4]heptan-5-yl)-5-chloro-6-fluoro-9H-pyrido[2,3-b]indol-8-yl) (methyl)carbamate and tert-butyl(3-bromo-4-((1R,3R)-1-((tert-butoxycarbonyl)amino)-5-azaspiro[2.4]heptan-5-yl)-5-chloro-6-fluoro-9H-pyrido[2,3-b]indol-8-yl)(methyl)carbamate and tert-butyl(3-bromo-4-((1S,3S)-1-((tert-butoxycarbonyl)amino)-5-azaspiro[2.4]heptan-5-yl)-5-chloro-6-fluoro-9H-pyrido[2,3-b]indol-8-yl)(methyl)carbamate

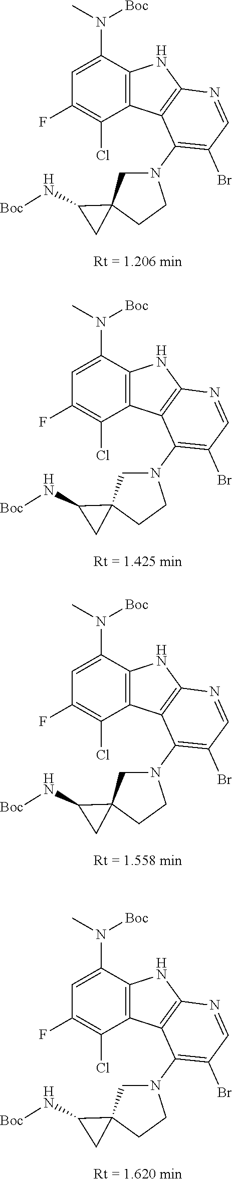 [SMILES: CN(C(=O)OC(C)(C)C)c1cc(F)c(Cl)c2c1[nH]c1ncc(Br)c(N3CC[C@@]4(C[C@@H]4NC(=O)OC(C)(C)C)C3)c12 ; CN(C(=O)OC(C)(C)C)c1cc(F)c(Cl)c2c1[nH]c1ncc(Br)c(N3CC[C@]4(C[C@H]4NC(=O)OC(C)(C)C)C3)c12 ; CN(C(=O)OC(C)(C)C)c1cc(F)c(Cl)c2c1[nH]c1ncc(Br)c(N3CC[C@@]4(C[C@H]4NC(=O)OC(C)(C)C)C3)c12 ; CN(C(=O)OC(C)(C)C)c1cc(F)c(Cl)c2c1[nH]c1ncc(Br)c(N3CC[C@]4(C[C@@H]4NC(=O)OC(C)(C)C)C3)c12]

Trans-tert-butyl N-[3-bromo-4-[2-(tert-butoxycarbonylamino)-5-azaspiro[2.4]heptan-5-yl]-5-chloro-6-fluoro-9H-pyrido[2,3-b]indol-8-yl]-N-methyl-carbamate (320 mg, 0.5 mmol) was subject to chiral SFC separation. The first collected eluent peak was concentrated in vacuo to give tert-butyl N-[3-bromo-4-[(2S,3R)-2-(tert-butoxycarbonylamino)-5-azaspiro[2.4]heptan-5-yl]-5-chloro-6-fluoro-9H-pyrido[2,3-b]indol-8-yl]-N-methyl-carbamate (peak 1′, Rt=1.206 min, 80 mg, Optical Rotation: (−)) as a yellow solid and the second collected eluent peak was concentrated in vacuo to give tert-butyl N-[3-bromo-4-[(2R,3S)-2-(tert-butoxycarbonylamino)-5-azaspiro[2.4]heptan-5-yl]-5-chloro-6-fluoro-9H-pyrido[2,3-b]indol-8-yl]-N-methyl-carbamate (peak 2′, Rt 1.425 min, 78 mg, Optical Rotation: (+)) as another yellow solid. SFC condition: Column: Daicel Chiralcel AS (250 mm×30 mm, 10 μm); Mobile Phase: 0.1% NH₃H₂O MeOH; Flow Rate (mL/min): 70 mL/min.

Cis-tert-butyl N-[3-bromo-4-[2-(tert-butoxycarbonylamino)-5-azaspiro[2.4]heptan-5-yl]-5-chloro-6-fluoro-9H-pyrido[2,3-b]indol-8-yl]-N-methyl-carbamate (710.0 mg, 1.11 mmol) was subject to chiral SFC separation. The first collected eluent peak was concentrated in vacuo to give tert-butyl N-[3-bromo-4-[(2R,3R)-2-(tert-butoxycarbonylamino)-5-azaspiro[2.4]heptan-5-yl]-5-chloro-6-fluoro-9H-pyrido[2,3-b]indol-8-yl]-N-methyl-carbamate (peak 3′, Rt=1.558 min, 360 mg, Optical Rotation: (−) as a yellow solid and the second collected eluent peak was concentrated in vacuo to give tert-butyl N-[3-bromo-4-[(2S,3S)-2-(tert-butoxycarbonylamino)-5-azaspiro[2.4]heptan-5-yl]-5-chloro-6-fluoro-9H-pyrido[2,3-b]indol-8-yl]-N-methyl-carbamate (peak 4′, Rt=1.620 min, 300 mg, Optical Rotation: (+)) as another yellow solid. SFC condition: Column: Daicel Chiralcel OD (250 mm×30 mm, 10 μm); Mobile Phase: 0.1% NH₃H₂O EtOH; Flowrate (mL/min): 50 mL/min.

### Step (c) Preparation of ethyl 6-[4-[(2R,3R)-2-(tert-butoxycarbonylamino)-5-azaspiro[2.4]heptan-5-yl]-8-[tert-butoxycarbonyl(methyl)amino]-5-chloro-6-fluoro-9H-pyrido[2,3-b]indol-3-yl]-1-methyl-4-oxo-1,8-naphthyridine-3-carboxylate

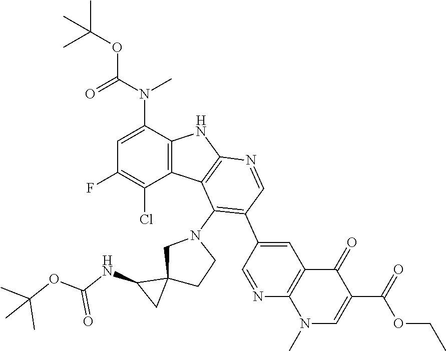 [SMILES: CCOC(=O)c1cn(C)c2ncc(-c3cnc4[nH]c5c(N(C)C(=O)OC(C)(C)C)cc(F)c(Cl)c5c4c3N3CC[C@@]4(C[C@H]4NC(=O)OC(C)(C)C)C3)cc2c1=O]

To a solution of tert-butyl N-[3-bromo-4-[(2R,3R)-2-(tert-butoxycarbonylamino)-5-azaspiro[2.4]heptan-5-yl]-5-chloro-6-fluoro-9H-pyrido[2,3-b]indol-8-yl]-N-methyl-carbamate (100 mg, 0.16 mmol) in TRF (1 mL) and water (0.01 mL) were added ethyl 1-methyl-4-oxo-6-(4,4,5,5-tetramethyl-1,3,2-dioxaborolan-2-yl)-1,4-dihydro-1,8-naphthyridine-3-carboxylate (86.41 mg, 0.310 mmol, Intermediate B1), Pd-Ad₂nBuP Biphenyl Precat (10.92 mg, 0.02 mmol), and K₃PO₄ (75.4 mg, 0.310 mmol) in one portion in a glove box under Argon atmosphere, and the resulting solution was stirred at 70° C. for 16 h under Ar. After cooled back to r.t., the reaction mixture was diluted with water (10 mL) and extracted with EtOAc (10 mL) three times. Combined organics were washed with brine (20 mL), dried over anhy. Na₂SO₄, filtered, and concentrated in vacuo to give a crude product, which was purified by prep-TLC (DCM/MeOH=10/1) to give ethyl 6-[4-[(2R,3R)-2-(tert-butoxycarbonylamino)-5-azaspiro[2.4]heptan-5-yl]-8-[tert-butoxycarbonyl(methyl)amino]-5-chloro-6-fluoro-9H-pyrido[2,3-b]indol-3-yl]-1-methyl-4-oxo-1,8-naphthyridine-3-carboxylate (62 mg, 50.13% yield) as a yellow solid. MS (ESI): 790.5 [{³⁵Cl} (M+H)⁺].

### Step (d) Preparation of 6-[4-[(2R,3R)-2-(tert-butoxycarbonylamino)-5-azaspiro[2.4]heptan-5-yl]-8-[tert-butoxycarbonyl(methyl)amino]-5-chloro-6-fluoro-9H-pyrido[2,3-b]indol-3-yl]-1-methyl-4-oxo-1,8-naphthyridine-3-carboxylic acid

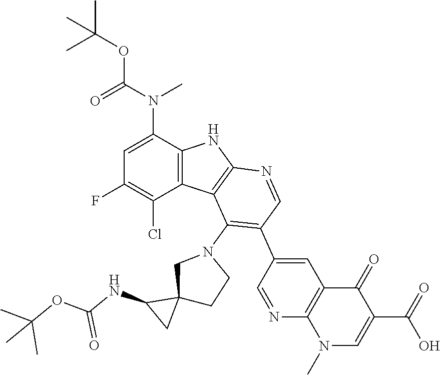 [SMILES: CN(C(=O)OC(C)(C)C)c1cc(F)c(Cl)c2c1[nH]c1ncc(-c3cnc4c(c3)c(=O)c(C(=O)O)cn4C)c(N3CC[C@@]4(C[C@H]4NC(=O)OC(C)(C)C)C3)c12]

To a solution of ethyl 6-[4-[(2R,3R)-2-(tert-butoxycarbonylamino)-5-azaspiro[2.4]heptan-5-yl]-8-[tert-butoxycarbonyl(methyl)amino]-5-chloro-6-fluoro-9H-pyrido[2,3-b]indol-3-yl]-1-methyl-4-oxo-1,8-naphthyridine-3-carboxylate (62 mg, 0.08 mmol) in EtOH (0.5 mL) and water (0.5 mL) was added NaOH (31 mg, 0.78 mmol) in one portion, and the resulting solution was stirred at 20° C. for 1 h. The mixture was concentrated under reduced pressure to give a residue, which was acidified with aq. HCl solution (1N) to pH=5-6, and extracted with EtOAc (30 mL) two times. The aqueous phase was extracted with additional EtOAc (10 mL) three times. Combined organics were dried over anhy. Na₂SO₄, filtered, and concentrated in vacuo to give 6-[4-[(2R,3R)-2-(tert-butoxycarbonylamino)-5-azaspiro[2.4]heptan-5-yl]-8-[tert-butoxycarbonyl(methyl)amino]-5-chloro-6-fluoro-9H-pyrido[2,3-b]indol-3-yl]-1-methyl-4-oxo-1,8-naphthyridine-3-carboxylic acid (55 mg, 92% yield) as a yellow solid. MS obsd. MS (ESI): 762.4 [{³⁵Cl} (M+H)⁺].

### Step (e) Preparation of 6-[4-[(2R,3R)-2-amino-5-azaspiro[2.4]heptan-5-yl]-5-chloro-6-fluoro-8-(methylamino)-9H-pyrido[2,3-b]indol-3-yl]-1-methyl-4-oxo-1,8-naphthyridine-3-carboxylic acid

To a solution of 6-[4-[(2R,3R)-2-(tert-butoxycarbonylamino)-5-azaspiro[2.4]heptan-5-yl]-8-[tert-butoxycarbonyl(methyl)amino]-5-chloro-6-fluoro-9H-pyrido[2,3-b]indol-3-yl]-1-methyl-4-oxo-1,8-naphthyridine-3-carboxylic acid (55 mg, 0.07 mmol) in DCM (0.5 mL) was added TFA (0.06 mL), and the solution was stirred at 20° C. for 1 h. The reaction mixture was concentrated in vacuo to give a crude product, which was purified by Prep-HPLC (water (1% TFA)-ACN) to give 6-[4-[(2R,3R)-2-amino-5-azaspiro[2.4]heptan-5-yl]-5-chloro-6-fluoro-8-(methylamino)-9H-pyrido[2,3-b]indol-3-yl]-1-methyl-4-oxo-1,8-naphthyridine-3-carboxylic acid (22.4 mg, 57.1% yield) as a green solid. MS (ESI): 562.4 [{³⁵Cl}(M+H)⁺]. ¹H NMR (400 MHz, DMSO) δppm: 14.72 (m, 1H), 11.91 (s, 1H), 9.31 (s, 1H), 9.12 (d, 1H), 8.71 (d, 1H), 8.24 (s, 1H), 7.99 (brs, 3H), 6.65 (d, 1H), 4.18 (s, 3H), 3.01-3.19 (m, 2H), 2.92 (s, 3H), 2.63 (m, 2H), 2.52 (m, 3H), 1.66 (m, 1H), 0.99 (m, 1H).

### Example 4.02

### 6-[4-[(2S,3S)-2-amino-5-azaspiro[2.4]heptan-5-yl]-5-chloro-6-fluoro-8-(methylamino)-9H-pyrido[2,3-b]indol-3-yl]-1-methyl-4-oxo-1,8-naphthyridine-3-carboxylic acid

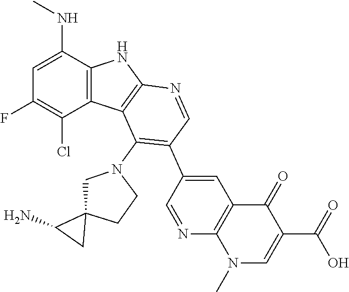 [SMILES: CNc1cc(F)c(Cl)c2c1[nH]c1ncc(-c3cnc4c(c3)c(=O)c(C(=O)O)cn4C)c(N3CC[C@]4(C[C@@H]4N)C3)c12]

The title compound was prepared in analogy to Example 4.01 by replacing tert-butyl N-[3-bromo-4-[(2R,3R)-2-(tert-butoxycarbonylamino)-5-azaspiro[2.4]heptan-5-yl]-5-chloro-6-fluoro-9H-pyrido[2,3-b]indol-8-yl]-N-methyl-carbamate with tert-butyl N-[3-bromo-4-[(2S,3S)-2-(tert-butoxycarbonylamino)-5-azaspiro[2.4]heptan-5-yl]-5-chloro-6-fluoro-9H-pyrido[2,3-b]indol-8-yl]-N-methyl-carbamate in step (c). MS (ESI): 562.3 [{³⁵Cl}(M+H)⁺]. ¹H NMR (400 MHz, DMSO) δ ppm: 14.72 (m, 1H), 11.85 (s, 1H), 9.31 (s, 1H), 9.12 (d, 1H), 8.71 (d, 1H), 8.19 (s, 1H), 7.94 (brs, 3H), 6.63 (d, 1H), 4.18 (s, 3H), 3.01-3.19 (m, 2H), 2.92 (s, 3H), 2.63 (m, 2H), 2.52 (m, 3H), 1.64 (m, 1H), 0.97 (m, 1H).

### Example 4.03

### 6-[4-[(2S,3R)-2-amino-5-azaspiro[2.4]heptan-5-yl]-5-chloro-6-fluoro-8-(methylamino)-9H-pyrido[2,3-b]indol-3-yl]-1-methyl-4-oxo-1,8-naphthyridine-3-carboxylic acid

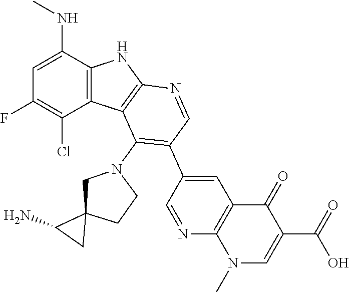 [SMILES: CNc1cc(F)c(Cl)c2c1[nH]c1ncc(-c3cnc4c(c3)c(=O)c(C(=O)O)cn4C)c(N3CC[C@@]4(C[C@@H]4N)C3)c12]

The title compound was prepared in analogy to Example 4.01 by replacing tert-butyl N-[3-bromo-4-[(2R,3R)-2-(tert-butoxycarbonylamino)-5-azaspiro[2.4]heptan-5-yl]-5-chloro-6-fluoro-9H-pyrido[2,3-b]indol-8-yl]-N-methyl-carbamate with tert-butyl N-[3-bromo-4-[(2S,3R)-2-(tert-butoxycarbonylamino)-5-azaspiro[2.4]heptan-5-yl]-5-chloro-6-fluoro-9H-pyrido[2,3-b]indol-8-yl]-N-methyl-carbamate in step (c). MS (ESI): 562.1 [{³⁵Cl}(M+H)⁺]. ¹H NMR (400 MHz, DMSO) δ ppm: 14.75 (brs, 1H), 11.89 (s, 1H), 9.29 (s, 1H), 9.11 (s, 1H), 8.68 (d, 1H), 8.22-8.13 (m, 4H), 6.64-6.61 (d, 1H), 4.17 (m, 3H), 3.21-3.14 (m, 2H), 2.91 (m, 4H), 1.92 (m, 2H), 0.83 (m, 2H).

### Example 4.04

### 6-[4-[(2R,3S)-2-amino-5-azaspiro[2.4]heptan-5-yl]-5-chloro-6-fluoro-8-(methylamino)-9H-pyrido[2,3-b]indol-3-yl]-1-methyl-4-oxo-1,8-naphthyridine-3-carboxylic acid

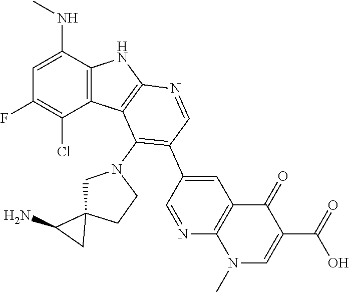 [SMILES: CNc1cc(F)c(Cl)c2c1[nH]c1ncc(-c3cnc4c(c3)c(=O)c(C(=O)O)cn4C)c(N3CC[C@]4(C[C@H]4N)C3)c12]

The title compound was prepared in analogy to Example 4.01 by replacing tert-butyl N-[3-bromo-4-[(2R,3R)-2-(tert-butoxycarbonylamino)-5-azaspiro[2.4]heptan-5-yl]-5-chloro-6-fluoro-9H-pyrido[2,3-b]indol-8-yl]-N-methyl-carbamate with tert-butyl N-[3-bromo-4-[(2R,3S)-2-(tert-butoxycarbonylamino)-5-azaspiro[2.4]heptan-5-yl]-5-chloro-6-fluoro-9H-pyrido[2,3-b]indol-8-yl]-N-methyl-carbamate in step (c). MS (ESI): 562.0 [{³⁵Cl}(M+H)⁺]. ¹H NMR (400 MHz, DMSO) δ ppm: 14.75 (brs, 1H), 11.86 (s, 1H), 9.29 (s, 1H), 9.11 (s, 1H), 8.68 (d, 1H), 8.19-8.12 (m, 4H), 6.64-6.61 (d, 1H), 4.17 (m, 3H), 3.21-3.14 (m, 2H), 2.91 (m, 4H), 1.92 (m, 2H), 0.83 (m, 2H).

### Example 5.01

### 6-[4-[(2S,3R)-2-Amino-5-azaspiro[2.4]heptan-5-yl]-5-cyano-6-fluoro-8-(methylamino)-9H-pyrido[2,3-b]indol-3-yl]-1-methyl-4-oxo-1,8-naphthyridine-3-carboxylic acid

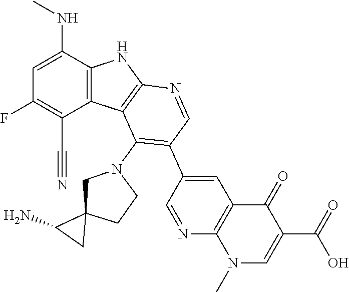 [SMILES: CNc1cc(F)c(C#N)c2c1[nH]c1ncc(-c3cnc4c(c3)c(=O)c(C(=O)O)cn4C)c(N3CC[C@@]4(C[C@@H]4N)C3)c12]

### Step (a) Preparation of trans-tert-butyl(4-(1-((tert-butoxycarbonyl)amino)-5-azaspiro[2.4]heptan-5-yl)-3-chloro-5-cyano-6-fluoro-9H-pyrido[2,3-b]indol-8-yl)(methyl)carbamate

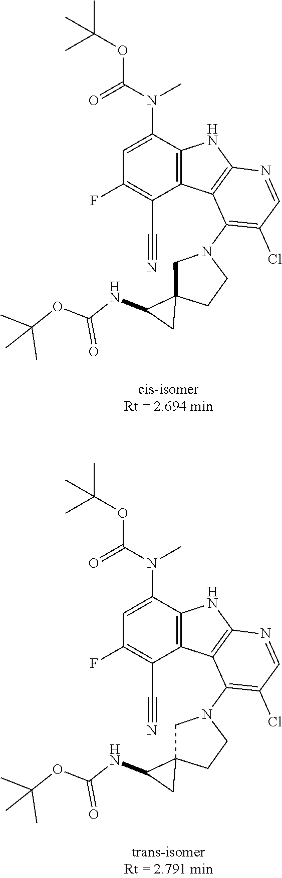

A solution of mixture of tert-butyl (3,4-dichloro-5-cyano-6-fluoro-9H-pyrido[2,3-b]indol-8-yl)(methyl)carbamate (Intermediate A4, 400.0 mg, 0.980 mmol, Intermediate A4), tert-butyl N-(5-azaspiro[2.4]heptan-2-yl)carbamate hydrochloride (486.27 mg, 1.95 mmol), and DIPEA (1.02 mL, 5.86 mmol) in NMP (3 mL) was stirred at 110° C. for 5 h. After cooled back to r.t., the reaction mixture was concentrated in vacuo and the residue was purified by Prep-HPLC (TFA as additive) to give cis-tert-butyl (4-(1-((tert-butoxycarbonyl)amino)-5-azaspiro[2.4]heptan-5-yl)-3-chloro-5-cyano-6-fluoro-9H-pyrido[2,3-b]indol-8-yl)(methyl)carbamate (220 mg, 37.2% yield) as a yellow solid and trans-tert-butyl (4-(1-((tert-butoxycarbonyl)amino)-5-azaspiro[2.4]heptan-5-yl)-3-chloro-5-cyano-6-fluoro-9H-pyrido[2,3-b]indol-8-yl)(methyl)carbamate (280 mg, 47.4% yield) as another yellow solid. MS (ESI): 585.3 ([{350}M+H]⁺), 587.3 ([{37Cl}+H]⁺). ¹H NMR (400 MHz, CHLOROFORM-d) δ ppm 8.53 (s, 1H), 7.24 (d, J=10.16 Hz, 1H), 3.72-3.97 (m, 2H), 3.53-3.65 (m, 1H), 3.44 (s, 3H), 2.66 (s, 1H), 2.08-2.48 (m, 2H), 1.53-1.91 (m, 2H), 1.38-1.52 (m, 18H), 1.14-1.22 (m, 1H).

### Step (b) Preparation of tert-butyl N-[4-[(2S,3R)-2-(tert-butoxycarbonylamino)-5-azaspiro[2.4]heptan-5-yl]-3-chloro-5-cyano-6-fluoro-9H-pyrido[2,3-b]indol-8-yl]-N-methyl-carbamate

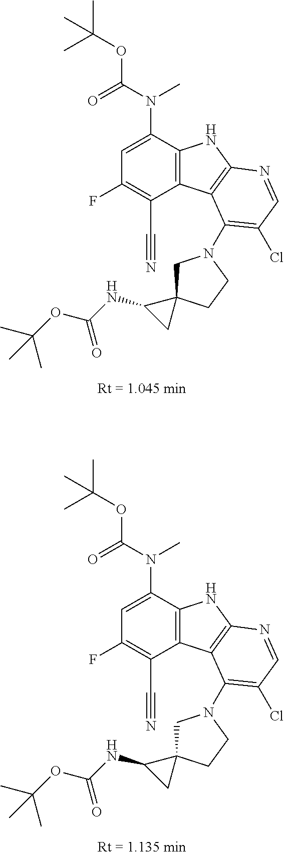 [SMILES: CN(C(=O)OC(C)(C)C)c1cc(F)c(C#N)c2c1[nH]c1ncc(Cl)c(N3CC[C@@]4(C[C@@H]4NC(=O)OC(C)(C)C)C3)c12 ; CN(C(=O)OC(C)(C)C)c1cc(F)c(C#N)c2c1[nH]c1ncc(Cl)c(N3CC[C@]4(C[C@H]4NC(=O)OC(C)(C)C)C3)c12]

Trans-tert-butyl (4-(1-((tert-butoxycarbonyl)amino)-5-azaspiro[2.4]heptan-5-yl)-3-chloro-5-cyano-6-fluoro-9H-pyrido[2,3-b]indol-8-yl)(methyl)carbamate (280.0 mg, 0.480 mmol) was separated by chiral SFC to give tert-butyl N-[4-[(2S,3R)-2-(tert-butoxycarbonylamino)-5-azaspiro[2.4]heptan-5-yl]-3-chloro-5-cyano-6-fluoro-9H-pyrido[2,3-b]indol-8-yl]-N-methyl-carbamate (55 mg, 18.7% yield) as a yellow solid and tert-butyl N-[4-[(2R,3S)-2-(tert-butoxycarbonylamino)-5-azaspiro[2.4]heptan-5-yl]-3-chloro-5-cyano-6-fluoro-9H-pyrido[2,3-b]indol-8-yl]-N-methyl-carbamate (40 mg, 13.6% yield) as another yellow solid. MS (ESI): 585.3 ([M+H]⁺)

SFC condition: Column: Chiralpak OJ (250 mm×30 mm, 10 μm); Mobile Phase: 0.1% NH₄OH MeOH; Flow Rate (mL/min): 50 mL/min.

### Step (c) Preparation of ethyl 6-[4-[(2S,3R)-2-(tert-butoxycarbonylamino)-5-azaspiro[2.4]heptan-5-yl]-8-[tert-butoxycarbonyl(methyl)amino]-5-cyano-6-fluoro-9H-pyrido[2,3-b]indol-3-yl]-1-methyl-4-oxo-1,8-naphthyridine-3-carboxylate

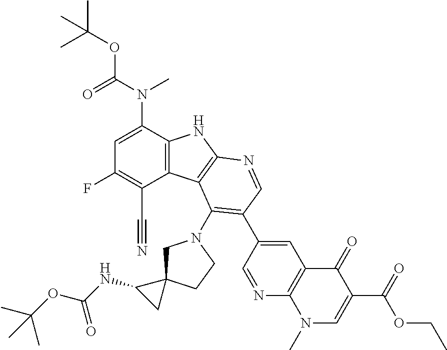 [SMILES: CCOC(=O)c1cn(C)c2ncc(-c3cnc4[nH]c5c(N(C)C(=O)OC(C)(C)C)cc(F)c(C#N)c5c4c3N3CC[C@@]4(C[C@@H]4NC(=O)OC(C)(C)C)C3)cc2c1=O]

A solution of mixture of tert-butyl N-[4-[(2S,3R)-2-(tert-butoxycarbonylamino)-5-azaspiro[2.4]heptan-5-yl]-3-chloro-5-cyano-6-fluoro-9H-pyrido[2,3-b]indol-8-yl]-N-methyl-carbamate (35.0 mg, 0.060 mmol), ethyl 1-methyl-4-oxo-6-(4,4,5,5-tetramethyl-1,3,2-dioxaborolan-2-yl)-1,4,4a,8a-tetrahydro-1,8-naphthyridine-3-carboxylate (32.14 mg, 0.09 mmol), Pd-Ad₂nBuP Biphenyl Precat (CAS: 1375477-29-4, 8.0 mg, 0.01 mmol), and K₃PO₄ (38.09 mg, 0.180 mmol, pre-dissolved in water (0.05 mL) in THF (0.5 mL) was stirred at 60° C. for 16 h under Ar. After LC-MS showed the starting material was consumed, the reaction mixture was cooled back to r.t. and subject to Prep-TLC separation (D CM/Me OH=10:1) to give ethyl 6-[4-[(2S,3R)-2-(tert-butoxycarbonylamino)-5-azaspiro[2.4]heptan-5-yl]-8-[tert-butoxycarbonyl(methyl)amino]-5-cyano-6-fluoro-9H-pyrido[2,3-b]indol-3-yl]-1-methyl-4-oxo-1,8-naphthyridine-3-carboxylate (30 mg, 55.9% yield) as a yellow solid. MS (ESI): 781.4 ([M+H]⁺). ¹H NMR (400 MHz, CHLOROFORM-d) δ ppm 8.90 (s, 1H), 8.81 (d, J=2.38 Hz, 1H), 8.72 (s, 1H), 8.23 (s, 1H), 7.18 (d, J=10.16 Hz, 1H), 5.30 (s, 2H), 5.14 (s, 1H), 4.37-4.46 (m, 3H), 4.06-4.10 (m, 3H), 3.99 (s, 1H), 3.40 (s, 3H), 1.38-1.47 (m, 14H), 1.33 (s, 9H), 0.75-0.91 (m, 2H).

### Step (d) Preparation of 6-[4-[(2S,3R)-2-(tert-butoxycarbonylamino)-5-azaspiro[2.4]heptan-5-yl]-8-[tert-butoxycarbonyl(methyl)amino]-5-cyano-6-fluoro-9H-pyrido[2,3-b]indol-3-yl]-1-methyl-4-oxo-1,8-naphthyridine-3-carboxylic acid

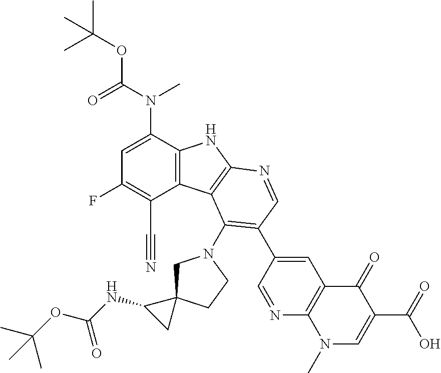 [SMILES: CN(C(=O)OC(C)(C)C)c1cc(F)c(C#N)c2c1[nH]c1ncc(-c3cnc4c(c3)c(=O)c(C(=O)O)cn4C)c(N3CC[C@@]4(C[C@@H]4NC(=O)OC(C)(C)C)C3)c12]

A solution of mixture of ethyl 6-[4-[(2S,3R)-2-(tert-butoxycarbonylamino)-5-azaspiro[2.4]heptan-5-yl]-8-[tert-butoxycarbonyl(methyl)amino]-5-cyano-6-fluoro-9H-pyrido[2,3-b]indol-3-yl]-1-methyl-4-oxo-1,8-naphthyridine-3-carboxylate (30 mg, 0.04 mmol), NaOH (61.47 mg, 1.54 mmol, pre-dissolved in water (0.2 mL) in TRF (2 mL) was stirred at 20° C. for 0.5 h. After LC-MS showed the starting material was consumed, the mixture was acidified with aq. HCl solution (2 N) to pH 4-5, diluted with water (10 mL), and extracted with DCM (20 mL) twice. Combined organics were dried over anhy. Na₂SO₄, filtered, and concentrated in vacuo to give 6-[4-[(2S,3R)-2-(tert-butoxycarbonylamino)-5-azaspiro[2.4]heptan-5-yl]-8-[tert-butoxycarbonyl(methyl)amino]-5-cyano-6-fluoro-9H-pyrido[2,3-b]indol-3-yl]-1-methyl-4-oxo-1,8-naphthyridine-3-carboxylic acid (28 mg, 95.8% yield) as a yellow solid. MS (ESI): 753.4 ([M+H]⁺). It was used directly in the next step without further purification. MS (ESI): 753.4 ([M+H]⁺).

### Step (d) Preparation of 6-[4-[(2S,3R)-2-amino-5-azaspiro[2.4]heptan-5-yl]-5-cyano-6-fluoro-8-(methylamino)-9H-pyrido[2,3-b]indol-3-yl]-1-methyl-4-oxo-1,8-naphthyridine-3-carboxylic acid

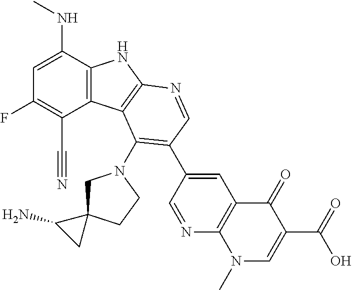 [SMILES: CNc1cc(F)c(C#N)c2c1[nH]c1ncc(-c3cnc4c(c3)c(=O)c(C(=O)O)cn4C)c(N3CC[C@@]4(C[C@@H]4N)C3)c12]

A solution of 6-[4-[(2S,3R)-2-(tert-butoxycarbonylamino)-5-azaspiro[2.4]heptan-5-yl]-8-[tert-butoxycarbonyl(methyl)amino]-5-cyano-6-fluoro-9H-pyrido[2,3-b]indol-3-yl]-1-methyl-4-oxo-1,8-naphthyridine-3-carboxylic acid (28 mg, 0.04 mmol) and TFA (0.11 mL, 1.49 mmol in DCM (2 mL) was stirred at 20° C. for 0.5 h. After LC-MS showed the starting material was consumed, the mixture was concentrated in vacuo to give a crude product, which was then purified by Prep-HPLC (TFA as additive) to give 6-[4-[(2S,3R)-2-amino-5-azaspiro[2.4]heptan-5-yl]-5-cyano-6-fluoro-8-(methylamino)-9H-pyrido[2,3-b]indol-3-yl]-1-methyl-4-oxo-1,8-naphthyridine-3-carboxylic acid (11 mg, 50% yield) as a yellow solid. MS (ESI): 553.3 ([M+H]⁺). ¹H NMR (400 MHz, DMSO-d6) δ ppm 12.06 (s, 1H), 9.32 (s, 1H), 9.13 (d, J=2.13 Hz, 1H), 8.70 (d, J=2.13 Hz, 1H), 8.03-8.31 (m, 4H), 6.98 (s, 1H), 6.68 (d, J=13.05 Hz, 1H), 4.20 (s, 3H), 3.44-3.56 (m, 3H), 3.01 (s, 3H), 2.55 (s, 2H), 1.83-2.20 (m, 2H), 0.65-0.91 (m, 2H).

### Example 5.02

### 6-[4-[(2R,3S)-2-amino-5-azaspiro[2.4]heptan-5-yl]-5-cyano-6-fluoro-8-(methylamino)-9H-pyrido[2,3-b]indol-3-yl]-1-methyl-4-oxo-1,8-naphthyridine-3-carboxylic acid

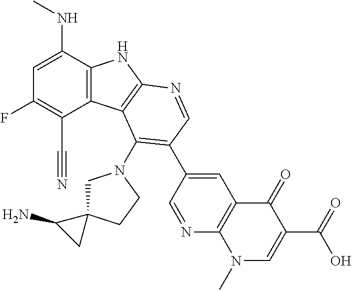 [SMILES: CNc1cc(F)c(C#N)c2c1[nH]c1ncc(-c3cnc4c(c3)c(=O)c(C(=O)O)cn4C)c(N3CC[C@]4(C[C@H]4N)C3)c12]

The title compound was prepared in analogy to Example 5.01 by replacing tert-butyl N-[4-[(2S,3R)-2-(tert-butoxycarbonylamino)-5-azaspiro[2.4]heptan-5-yl]-3-chloro-5-cyano-6-fluoro-9H-pyrido[2,3-b]indol-8-yl]-N-methyl-carbamate with tert-butyl N-[4-[(2R,3S)-2-(tert-butoxycarbonylamino)-5-azaspiro[2.4]heptan-5-yl]-3-chloro-5-cyano-6-fluoro-9H-pyrido[2,3-b]indol-8-yl]-N-methyl-carbamate in step (c). MS (ESI): 553.3 ([M+H]⁺). ¹H NMR (400 MHz, DMSO) δppm 11.99 (s, 1H), 9.32 (s, 1H), 9.13 (d, J=2.13 Hz, 1H), 8.70 (d, J=2.26 Hz, 1H), 8.22 (s, 1H), 8.08 (s, 3H), 6.90 (s, 1H), 6.69 (d, J=13.05 Hz, 1H), 4.20 (s, 3H), 3.23-3.50 (m, 3H), 3.01 (s, 3H), 2.04 (s, 2H), 0.66-0.88 (m, 2H).

### Example 5.03

### 6-[4-[(2S,3S)-2-amino-5-azaspiro[2.4]heptan-5-yl]-5-cyano-6-fluoro-8-(methylamino)-9H-pyrido[2,3-b]indol-3-yl]-1-methyl-4-oxo-1,8-naphthyridine-3-carboxylic acid

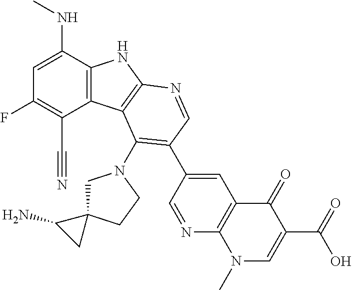 [SMILES: CNc1cc(F)c(C#N)c2c1[nH]c1ncc(-c3cnc4c(c3)c(=O)c(C(=O)O)cn4C)c(N3CC[C@]4(C[C@@H]4N)C3)c12]

The title compound was prepared in analogy to Example 5.01 by replacing tert-butyl N-[4-[(2S,3R)-2-(tert-butoxycarbonylamino)-5-azaspiro[2.4]heptan-5-yl]-3-chloro-5-cyano-6-fluoro-9H-pyrido[2,3-b]indol-8-yl]-N-methyl-carbamate with tert-butyl N-[4-[(2S,3S)-2-(tert-butoxycarbonylamino)-5-azaspiro[2.4]heptan-5-yl]-3-chloro-5-cyano-6-fluoro-9H-pyrido[2,3-b]indol-8-yl]-N-methyl-carbamate in step (c). MS (ESI): 553.3 ([M+H]⁺). ¹H NMR (400 MHz, MeOD) δ ppm 9.20 (s, 1H), 9.15 (s, 1H), 8.86 (s, 1H), 8.23 (m, 1H), 6.62˜6.66 (m, 1H), 4.24(m, 3H), 3.82˜3.50 (m, 6H), 3.10 (s, 3H), 2.68˜2.71 (m, 1H), 1.95˜2.05 (m, 1H), 1.14 (m, 1H).

### Example 5.04

### 6-[4-[(2R,3R)-2-amino-5-azaspiro[2.4]heptan-5-yl]-5-cyano-6-fluoro-8-(methylamino)-9H-pyrido[2,3-b]indol-3-yl]-1-methyl-4-oxo-1,8-naphthyridine-3-carboxylic acid

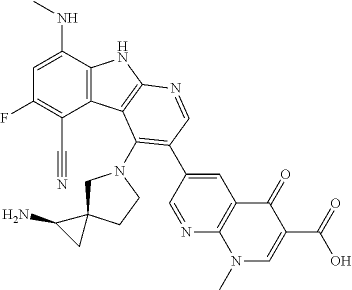 [SMILES: CNc1cc(F)c(C#N)c2c1[nH]c1ncc(-c3cnc4c(c3)c(=O)c(C(=O)O)cn4C)c(N3CC[C@@]4(C[C@H]4N)C3)c12]

The title compound was prepared in analogy to Example 5.01 by replacing tert-butyl N-[4-[(2S,3R)-2-(tert-butoxycarbonylamino)-5-azaspiro[2.4]heptan-5-yl]-3-chloro-5-cyano-6-fluoro-9H-pyrido[2,3-b]indol-8-yl]-N-methyl-carbamate with tert-butyl N-[4-[(2R,3R)-2-(tert-butoxycarbonylamino)-5-azaspiro[2.4]heptan-5-yl]-3-chloro-5-cyano-6-fluoro-9H-pyrido[2,3-b]indol-8-yl]-N-methyl-carbamate (c). MS (ESI): 553.1 ([M+H]⁺). ¹H NMR (400 MHz, DMSO) δ 11.99 (br s, 1H), 9.30 (s, 1H), 9.13 (d, J=2.3 Hz, 1H), 8.72 (d, J=2.0 Hz, 1H), 8.21 (s, 1H), 7.96 (br s, 3H), 6.89 (br s, 1H), 6.68 (d, J=13.1 Hz, 1H), 4.18 (s, 3H), 3.54-3.49 (m, 4H), 3.26 (br d, J=8.4 Hz, 1H), 3.00 (br s, 3H), 2.62 (br s, 1H), 1.62 (br s, 1H), 1.75-1.49 (m, 1H), 1.05 (t, J=7.0 Hz, 2H), 1.12-0.99 (m, 1H), 0.95 (br s, 1H).

### Example 6.01

### 6-[4-[(2R,3R)-2-Amino-5-azaspiro[2.5]octan-5-yl]-5,6-difluoro-8-(methylamino)-9H-pyrido[2,3-b]indol-3-yl]-1-methyl-4-oxo-1,8-naphthyridine-3-carboxylic acid

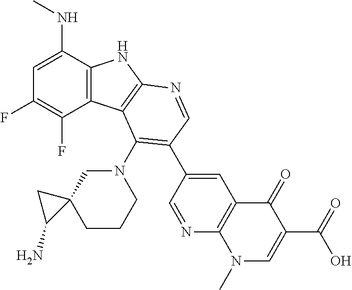 [SMILES: CNc1cc(F)c(F)c2c1[nH]c1ncc(-c3cnc4c(c3)c(=O)c(C(=O)O)cn4C)c(N3CCC[C@@]4(C[C@H]4N)C3)c12]

Step (a) Preparation of cis-tert-butyl N-[4-[2-(tert-butoxycarbonylamino)-5-azaspiro[2.5]octan-5-yl]-3-chloro-5,6-difluoro-9H-pyrido[2,3-b]indol-8-yl]-N-methyl-carbamate

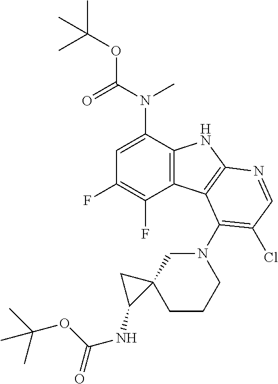 [SMILES: CN(C(=O)OC(C)(C)C)c1cc(F)c(F)c2c1[nH]c1ncc(Cl)c(N3CCC[C@@]4(C[C@H]4NC(=O)OC(C)(C)C)C3)c12]

A solution of cis-tert-butyl N-(5-azaspiro[2.5]octan-2-yl)carbamate (400 mg, 1.77 mmol) in NMP (5 mL) was added tert-butyl N-(3,4-dichloro-5,6-difluoro-9H-pyrido[2,3-b]indol-8-yl)-N-methyl-carbamate (500 mg, 1.24 mmol, Intermediate A2) and DIPEA (1.3 mL, 7.46 mmol) in NMP (3 mL) was stirred at 120° C. for 18 h. After TLC (petroleum ether:EtOAc=2:1) showed the reaction was completed, the solution was purified by prep-HPLC (TFA) to give cis-tert-Butyl N-[4-[2-(tert-butoxycarbonylamino)-5-azaspiro[2.5]octan-5-yl]-3-chloro-5,6-difluoro-9H-pyrido[2,3-b]indol-8-yl]-N-methyl-carbamate (400 mg, 54.4% yield) as a white solid. MS (ESI): 543.4 ({³⁵Cl} M+H)⁺, 545.4 ({³⁷Cl} M+H)⁺.

### Step (b) Preparation of tert-butyl N-[4-[(2S,3S)-2-(tert-butoxycarbonylamino)-5-azaspiro[2.5]octan-5-yl]-3-chloro-5,6-difluoro-9H-pyrido[2,3-b]indol-8-yl]-N-methyl-carbamate and tert-butyl N-[4-[(2R,3R)-2-(tert-butoxycarbonylamino)-5-azaspiro[2.5]octan-5-yl]-3-chloro-5,6-difluoro-9H-pyrido[2,3-b]indol-8-yl]-N-methyl-carbamate

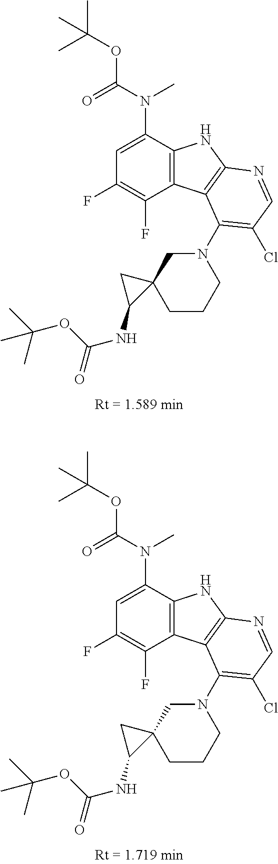 [SMILES: CN(C(=O)OC(C)(C)C)c1cc(F)c(F)c2c1[nH]c1ncc(Cl)c(N3CCC[C@]4(C[C@@H]4NC(=O)OC(C)(C)C)C3)c12 ; CN(C(=O)OC(C)(C)C)c1cc(F)c(F)c2c1[nH]c1ncc(Cl)c(N3CCC[C@@]4(C[C@H]4NC(=O)OC(C)(C)C)C3)c12]

SFC separation of cis-(4-(1-((tert-butoxycarbonyl)amino)-5-azaspiro[2.5]octan-5-yl)-3-chloro-5,6-difluoro-9H-pyrido[2,3-b]indol-8-yl)(methyl)carbamate (400.0 mg, 0.680 mmol) afforded tert-butyl N-[4-[(2S,3S)-2-(tert-butoxycarbonylamino)-5-azaspiro[2.5]octan-5-yl]-3-chloro-5,6-difluoro-9H-pyrido[2,3-b]indol-8-yl]-N-methyl-carbamate (180 mg, 45% yield) and tert-butyl N-[4-[(2R,3R)-2-(tert-butoxycarbonylamino)-5-azaspiro[2.5]octan-5-yl]-3-chloro-5,6-difluoro-9H-pyrido[2,3-b]indol-8-yl]-N-methyl-carbamate (160 mg, 40% yield) as white solids. MS: 543.4 ({³⁵Cl} M+H)⁺, 545.4 ({³⁷Cl} M+H)⁺.

SFC condition: Column: AD, 250×20 mm I.D., 5 μm. Mobile phase: A for CO₂ and B for IPA (0.1% NH₃H₂O); Gradient: B 20%. Flow rate: 50 mL/min. Back pressure: 100 bar. Column temperature: 35° C.

### Step (c) Preparation of ethyl 6-[4-[(2R,3R)-2-(tert-butoxycarbonylamino)-5-azaspiro[2.5]octan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylate

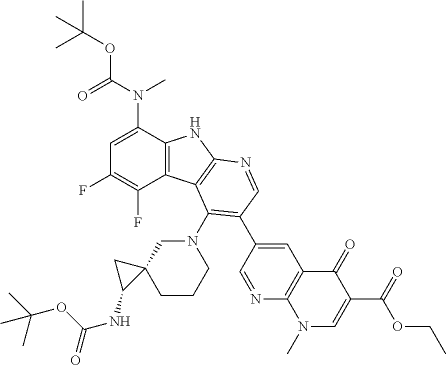 [SMILES: CCOC(=O)c1cn(C)c2ncc(-c3cnc4[nH]c5c(N(C)C(=O)OC(C)(C)C)cc(F)c(F)c5c4c3N3CCC[C@@]4(C[C@H]4NC(=O)OC(C)(C)C)C3)cc2c1=O]

A solution of K₃PO₄ (86.04 mg, 0.41 mmol), cataCXium A-Pd-G2 (18.07 mg, 0.03 mmol), tert-butyl N-[4-[(2R,3R)-2-(tert-butoxycarbonylamino)-5-azaspiro[2.5]octan-5-yl]-3-chloro-5,6-difluoro-9H-pyrido[2,3-b]indol-8-yl]-N-methyl-carbamate (80 mg, 0.14 mmol), and ethyl 1-methyl-4-oxo-6-(4,4,5,5-tetramethyl-1,3,2-dioxaborolan-2-yl)-1,8-naphthyridine-3-carboxylate (72.6 mg, 0.2 mmol, Intermediate B1) in water (1 mL) and TRF (20 mL) was stirred at 70° C. for 15 h under Ar. After LC-MS showed the starting material was consumed, the reaction mixture was filtered and the filtrate was concentrated under reduced pressure. The residue was purified by prep-HPLC (TFA as additive) to give ethyl 6-[4-[(2R,3R)-2-(tert-butoxycarbonylamino)-5-azaspiro[2.5]octan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylate (60 mg, 56.4% yield) as a brown solid. MS (ESI): 787.4 ([M+H]⁺).

### Step (d) Preparation of 6-[4-[(2R,3R)-2-(tert-butoxycarbonylamino)-5-azaspiro[2.5]octan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylic acid

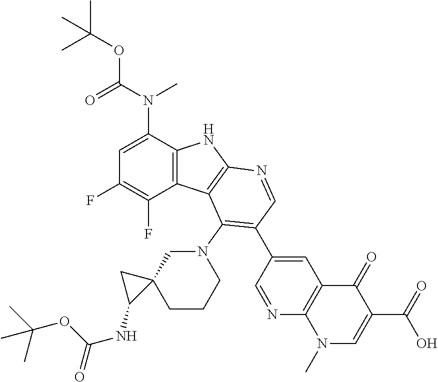 [SMILES: CN(C(=O)OC(C)(C)C)c1cc(F)c(F)c2c1[nH]c1ncc(-c3cnc4c(c3)c(=O)c(C(=O)O)cn4C)c(N3CCC[C@@]4(C[C@H]4NC(=O)OC(C)(C)C)C3)c12]

To a solution of ethyl 6-[4-[(2R,3R)-2-(tert-butoxycarbonylamino)-5-azaspiro[2.5]octan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylate (60 mg, 0.08 mmol) in TRF (1 mL) and water(0.2 mL) was added NaOH (30.46 mg, 0.760 mmol), and the resulting solution was stirred at 20° C. for 2 h. After LC-MS showed the starting material was consumed, the reaction mixture was concentrated in vacuo and the residue was acidified with 1 N HCl solution to pH=6 before subject to prep-HPLC (TFA as additive) purification to give 6-[4-[(2R,3R)-2-(tert-butoxycarbonylamino)-5-azaspiro[2.5]octan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylic acid (50 mg, 86.4% yield) as a yellow solid. MS (ESI): 766.4 ([M+H]⁺).

### Step (e) Preparation of 6-[4-[(2R,3R)-2-amino-5-azaspiro[2.5]octan-5-yl]-5,6-difluoro-8-(methylamino)-9H-pyrido[2,3-b]indol-3-yl]-1-methyl-4-oxo-1,8-naphthyridine-3-carboxylic acid

To a solution of 6-[4-[(2R,3R)-2-(tert-butoxycarbonylamino)-5-azaspiro[2.5]octan-5-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylic acid (50 mg, 0.07 mmol) in DCM (5 mL) was added TFA (2.86 mL, 11.44 mmol), and the resulting solution was stirred at 20° C. for 2 h. After LC-MS showed the starting material was consumed, the reaction mixture was concentrated in vacuo to give a crude product, which was purified by prep-HPLC(TFA as additive) to give 6-[4-[(2R,3R)-2-amino-5-azaspiro[2.5]octan-5-yl]-5,6-difluoro-8-(methylamino)-9H-pyrido[2,3-b]indol-3-yl]-1-methyl-4-oxo-1,8-naphthyridine-3-carboxylic acid (21.8 mg, 55.7% yield) as a yellow solid. ¹H NMR (400 MHz, DMSO-d₆) δ 11.96 (s, 1H), 9.28 (s, 1H), 9.09 (s, 1H), 8.90-8.59 (m, 1H), 8.40 (s, 1H), 7.91 (s, 3H), 6.63 (dd, J=6.2, 13.3 Hz, 1H), 4.18 (s, 3H), 3.41-3.34 (m, 1H), 3.10-2.95 (m, 1H), 2.90 (s, 3H), 2.54 (s, 3H), 2.33 (s, 1H), 1.74-1.29 (m, 3H), 0.98 (d, J=11.2 Hz, 1H), 0.56 (s, 1H), 0.16 (br s, 1H) MS (ESI): 560.2 ([M+H]⁺).

### Example 6.02

### 6-[4-[(2S,3S)-2-amino-5-azaspiro[2.5]octan-5-yl]-5,6-difluoro-8-(methyl amino)-9H-pyrido [2,3-b]indol-3-yl]-1-methyl-4-oxo-1,8-naphthyridine-3-carboxylic acid

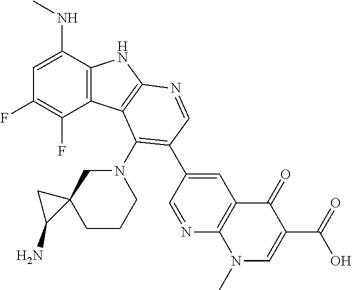 [SMILES: CNc1cc(F)c(F)c2c1[nH]c1ncc(-c3cnc4c(c3)c(=O)c(C(=O)O)cn4C)c(N3CCC[C@]4(C[C@@H]4N)C3)c12]

The title compound was prepared in analogy to Example 6.01 by replacing tert-butyl N-[4-[(2R,3R)-2-(tert-butoxycarbonylamino)-5-azaspiro[2.5]octan-5-yl]-3-chloro-5,6-difluoro-9H-pyrido[2,3-b]indol-8-yl]-N-methyl-carbamate with teat-Butyl N-[4-[(2S,3S)-2-(tert-butoxycarbonylamino)-5-azaspiro[2.5]octan-5-yl]-3-chloro-5,6-difluoro-9H-pyrido[2,3-b]indol-8-yl]-N-methyl-carbamate in step (b). MS (ESI): 560.3 ([M+H]⁺). ¹H NMR (400 MHz, DMSO-d6) δ ppm 11.77 (s, 1H) 9.10 (s, 1H), 8.90 (d, J=1.83 Hz, 1H), 8.60 (s, 1H), 8.20 (s, 1H), 7.70 (d, J=2.93 Hz, 3H), 6.44 (dd, J=13.33, 6.24 Hz, 1H), 3.99 (s, 3H), 3.07-3.69 (m, 2H), 2.77-3.03 (m, 1H), 2.71 (s, 3H), 2.34 (s, 1H), 2.13 (d, J=1.83 Hz, 1H), 1.04-1.60 (m, 3H), 0.79 (d, J=10.27 Hz, 1H), 0.38 (s, 1H), 0.00 (s, 1H).

### BIOLOGICAL EXAMPLES

### Example 7

### 50% Growth Inhibitory Concentration (IC₅₀) Determination Assay:

The in vitro antimicrobial activity of the compounds against S. aureus (ATCC29213), K. pneumoniae (ATCC10031), and A. baumannii (ATCC17978), was determined according to the following procedure:

The assay used a 10-points Iso-Sensitest broth medium to measure quantitatively the in vitro activity of the compounds against S. aureus ATCC29213, K. pneumoniae ATCC 10031, and A. baumannii ATCC17978.

Stock compounds in DMSO were serially two-fold diluted (range from 50 to 0.097 μM final concentration) in 384 wells microtiter plates and inoculated with 49 μL the bacterial suspension in Iso-Sensitest broth medium to have a final cell concentration of ˜5×10⁵ CFU/mL in a final volume/well of 50 μL/well. Microtiter plates were incubated at 35±2° C.

Bacterial cell growth was determined with the measurement of optical density at k=600 nm each 20 minutes over a time course of 16 h.

Growth inhibition was calculated during the logarithmic growth of the bacterial cells with determination of the concentration inhibiting 50% (IC₅₀) of the growth.

Compounds of the present invention were tested for their concentration inhibiting 50% (IC₅₀). The data of IC₅₀ over S. aureus (ATCC29213), K. pneumoniae (ATCC10031), and A. baumannii (ATCC17978) are illustrated in Table 6. Particular compounds of the present invention were found to have IC₅₀≤1 μM.

TABLE 2
IC₅₀ values of the compounds of this invention against
S. aureus, K. pneumoniae and A. baumannii

| | IC50 (μM) | | |
|---|---|---|---|
| Example | S. aureus | K. pneumoniae | A. baumannii |
| No. | ATCC29213 | ATCC10031 | ATCC17978 |
| 1.02 | 3.89 | <0.098* | 2.86 |
| 1.03 | 0.944 | <0.098 | 1.49 |
| 1.04 | 0.572 | 0.110 | 1.33 |
| 1.05 | <0.098 | N.A. | 0.602 |
| 1.06 | 0.342 | <0.098 | 2.48 |
| 1.07 | 1.67 | 0.429 | 4.79 |
| 1.08 | 0.314 | <0.098 | 0.654 |
| 1.09 | 0.513 | 0.250 | 3.72 |
| 1.11 | 0.294 | <0.098 | 3.62 |
| 1.12 | 0.448 | <0.098 | 0.889 |
| 1.13 | 0.151 | 0.128 | 2.79 |
| 1.16 | <0.098 | <0.098 | 0.507 |
| 1.17 | 0.532 | <0.098 | 2.95 |
| 2.01 | <0.098 | <0.098 | 0.155 |
| 2.02 | <0.098 | <0.098 | 0.261 |
| 3.04 | <0.098 | <0.098 | 0.175 |
| 4.01 | <0.098 | <0.098 | 0.0765 |
| 4.02 | <0.098 | <0.098 | 0.0973 |
| 4.03 | <0.098 | <0.098 | 0.0553 |
| 4.04 | <0.098 | <0.098 | 0.0793 |
| 5.01 | <0.098 | <0.098 | 0.477 |
| 5.02 | <0.098 | <0.098 | 0.417 |
| 5.03 | <0.098 | <0.098 | 0.497 |
| 6.01 | <0.098 | N.A. | N.A. |
| 6.02 | <0.098 | N.A. | N.A. |

“*”: the detection limit.

1. A compound of formula (I), 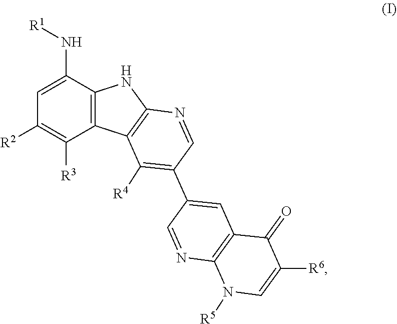
wherein
R¹ is C₁₋₆alkyl;
R² is halogen;
R³ is halogen or cyano;
R⁴ is selected from the group consisting of
1,3,4,5,6,6a-hexahydrocyclopenta[c]pyrrolyl substituted by amino;
2,3,3a,4,6,6a-hexahydropyrrolo[2,3-c]pyrrolyl substituted by amino;
3,3a,4,5,6,6a-hexahydro-1H-cyclopenta[c]pyrrolyl substituted by amino;
azabicyclo[3.1.0]hexanyl substituted by amino or aminoC₁₋₆alkyl;
azaspiro[2.4]heptanyl substituted by amino;
azaspiro[2.5]octanyl substituted by amino;
azaspiro[3.3]heptanyl substituted by amino;
piperidinyl substituted twice by aminoC₁₋₆alkyl and halogen; and
pyrrolidinyl substituted once or twice by substituents independently selected from amino, aminoC₁₋₆alkyl, aminoC₃₋₇cycloalkyl, haloC₁₋₆alkyl and halogen;
R⁵ is C₁₋₆alkyl; and
R⁶ is carboxy;
or a pharmaceutically acceptable salt thereof.

2. A compound according to claim 1, wherein
R¹ is methyl;
R² is fluoro;
R³ is chloro, fluoro or cyano;
R⁴ is aminocyclopropylpyrrolidinyl; amino(trifluoromethyl)pyrrolidinyl; amino-1,3,4,5,6,6a-hexahydrocyclopenta[c]pyrrolyl; amino-3,3a,4,5,6,6a-hexahydro-1H-cyclopenta[c]pyrrolyl; aminoazabicyclo[3.1.0]hexanyl; aminoazaspiro[2.4]heptanyl; aminoazaspiro[2.5]octanyl; aminoazaspiro[3.3]heptanyl; aminomethyl(fluoro)piperidinyl; aminomethyl(fluoro)pyrrolidinyl; aminomethylazabicyclo[3.1.0]hexanyl; aminomethylpyrrolidinyl; aminopropyl-2,3,3a,4,6,6a-hexahydropyrrolo[2,3-c]pyrrolyl; or aminopyrrolidinyl;
R⁵ is methyl or ethyl; and
R⁶ is carboxy;
or a pharmaceutically acceptable salt thereof.

3. A compound according to claim 2, or a pharmaceutically acceptable salt thereof, wherein R⁴ is azaspiro[2.4]heptanyl substituted by amino; or pyrrolidinyl substituted once or twice by substituents independently selected from amino, aminoC₁₋₆alkyl, aminoC₃₋₇cycloalkyl, haloC₁₋₆alkyl and halogen.

4. A compound according to claim 3, or a pharmaceutically acceptable salt thereof, wherein R⁴ is aminoazaspiro[2.4]heptanyl; aminocyclopropylpyrrolidinyl; amino(trifluoromethyl)pyrrolidinyl; aminomethyl(fluoro)pyrrolidinyl; aminomethylpyrrolidinyl; or aminopyrrolidinyl.

5. A compound according to claim 4, or a pharmaceutically acceptable salt thereof, wherein R⁵ is methyl.

6. A compound according to claim 1, wherein
R¹ is C₁₋₆alkyl;
R² is halogen;
R³ is halogen or cyano;
R⁴ is azaspiro[2.4]heptanyl substituted by amino; or
pyrrolidinyl substituted once or twice by substituents independently selected from amino, aminoC₁₋₆alkyl, aminoC₃₋₇cycloalkyl, haloC₁₋₆alkyl and halogen;
R⁵ is C₁₋₆alkyl; and
R⁶ is carboxy;
or a pharmaceutically acceptable salt thereof.

7. A compound according to claim 6, wherein
R¹ is methyl;
R² is chloro or fluoro;
R³ is chloro, fluoro or cyano;
R⁴ is aminoazaspiro[2.4]heptanyl; aminocyclopropylpyrrolidinyl;
amino(trifluoromethyl)pyrrolidinyl; aminomethyl(fluoro)pyrrolidinyl;
aminomethylpyrrolidinyl; or aminopyrrolidinyl;
R⁵ is methyl;
R⁶ is carboxy;
or a pharmaceutically acceptable salt thereof.

8. A compound selected from:
6-[4-[3-(1-Aminocyclopropyl)pyrrolidin-1-yl]-5,6-difluoro-8-(methylamino)-9H-pyrido[2,3-b]indol-3-yl]-1-methyl-4-oxo-1,8-naphthyridine-3-carboxylic acid;
6-[4-(7-Amino-2-azaspiro[3.3]heptan-2-yl)-5,6-difluoro-8-(methylamino)-9H-pyrido[2,3-b]indol-3-yl]-1-methyl-4-oxo-1,8-naphthyridine-3-carboxylic acid;
6-[4-(3-aminopyrrolidin-1-yl)-5,6-difluoro-8-(methylamino)-9H-pyrido[2,3-b]indol-3-yl]-1-ethyl-4-oxo-1,8-naphthyridine-3-carboxylic acid;
6-(4-(3-(Aminomethyl)pyrrolidin-1-yl)-5,6-difluoro-8-(methylamino)-9H-pyrido[2,3-b]indol-3-yl)-1-ethyl-4-oxo-1,4-dihydro-1,8-naphthyridine-3-carboxylic acid;
6-(4-(3-Amino-3-(trifluoromethyl)pyrrolidin-1-yl)-5,6-difluoro-8-(methyl amino)-9H-pyrido[2,3-b]indol-3-yl)-1-methyl-4-oxo-1,4-dihydro-1,8-naphthyridine-3-carboxylic acid;
6-[4-[1-(aminomethyl)-3-azabicyclo[3.1.0]hexan-3-yl]-5,6-difluoro-8-(methylamino)-9H-pyrido[2,3-b]indol-3-yl]-1-methyl-4-oxo-1,8-naphthyridine-3-carboxylic acid;
cis-6-[5,6-difluoro-8-(methylamino)-4-[5-(3-aminopropyl)-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[5,6-difluoro-8-(methylamino)-4-[rel-(1S,5R)-6-amino-3-azabicyclo[3.1.0]hexan-3-yl]-9H-pyrido[2,3-b]indol-3-yl]-1-methyl-4-oxo-1,8-naphthyridine-3-carboxylic acid;
6-[4-[2-(Aminomethyl)pyrrolidin-1-yl]-5,6-difluoro-8-(methylamino)-9H-pyrido[2,3-b]indol-3-yl]-1-methyl-4-oxo-1,8-naphthyridine-3-carboxylic acid;
6-[4-[(2S)-2-(Aminomethyl)pyrrolidin-1-yl]-5,6-difluoro-8-(methyl amino)-9H-pyrido[2,3-b]indol-3-yl]-1-methyl-4-oxo-1,8-naphthyridine-3-carboxylic acid;
6-[4-[3-(Aminomethyl)pyrrolidin-1-yl]-5,6-difluoro-8-(methylamino)-9H-pyrido[2,3-b]indol-3-yl]-1-methyl-4-oxo-1,8-naphthyridine-3-carboxylic acid;
trans-6-(5,6-Difluoro-8-(methylamino)-4-[3a-amino-1,3,4,5,6,6a-hexahydrocyclopenta[c]pyrrol-2-yl]-9H-pyrido[2,3-b]indol-3-yl)-1-methyl-4-oxo-1,8-naphthyridine-3-carboxylic acid;
6-[5-Cyano-6-fluoro-8-(methylamino)-4-[rel-(3aR,4R,6aS)-4-amino-3,3a,4,5,6,6a-hexahydro-1H-cyclopenta[c]pyrrol-2-yl]-9H-pyrido[2,3-b]indol-3-yl]-1-methyl-4-oxo-1,8-naphthyridine-3-carboxylic acid;
6-[4-[3-(Aminomethyl)-3-fluoro-pyrrolidin-1-yl]-5,6-difluoro-8-(methylamino)-9H-pyrido[2,3-b]indol-3-yl]-1-ethyl-4-oxo-1,8-naphthyridine-3-carboxylic acid;
6-[4-[3-(Aminomethyl)-3-fluoro-1-piperidyl]-5,6-difluoro-8-(methylamino)-9H-pyrido[2,3-b]indol-3-yl]-1-ethyl-4-oxo-1,8-naphthyridine-3-carboxylic acid;
6-[4-[3-(Aminomethyl)-3-fluoro-pyrrolidin-1-yl]-5,6-difluoro-8-(methylamino)-9H-pyrido[2,3-b]indol-3-yl]-1-methyl-4-oxo-1,8-naphthyridine-3-carboxylic acid;
6-[4-[(3R)-3-Aminopyrrolidin-1-yl]-5,6-difluoro-8-(methylamino)-9H-pyrido[2,3-b]indol-3-yl]-1-methyl-4-oxo-1,8-naphthyridine-3-carboxylic acid;
trans-6-[4-(2-amino-5-azaspiro[2.4]heptan-5-yl)-5,6-difluoro-8-(methylamino)-9H-pyrido[2,3-b]indol-3-yl]-1-methyl-4-oxo-1,8-naphthyridine-3-carboxylic acid;
cis-6-[4-(2-amino-5-azaspiro[2.4]heptan-5-yl)-5,6-difluoro-8-(methylamino)-9H-pyrido[2,3-b]indol-3-yl]-1-methyl-4-oxo-1,8-naphthyridine-3-carboxylic acid;
6-[4-[(2R,3R)-2-amino-5-azaspiro[2.4]heptan-5-yl]-5,6-difluoro-8-(methylamino)-9H-pyrido[2,3-b]indol-3-yl]-1-methyl-4-oxo-1,8-naphthyridine-3-carboxylic acid;
6-[4-[(25,3R)-2-amino-5-azaspiro[2.4]heptan-5-yl]-5,6-difluoro-8-(methylamino)-9H-pyrido[2,3-b]indol-3-yl]-1-methyl-4-oxo-1,8-naphthyridine-3-carboxylic acid;
6-[4-[(2R,3S)-2-amino-5-azaspiro[2.4]heptan-5-yl]-5,6-difluoro-8-(methylamino)-9H-pyrido[2,3-b]indol-3-yl]-1-methyl-4-oxo-1,8-naphthyridine-3-carboxylic acid;
6-[4-[(2S,3S)-2-amino-5-azaspiro[2.4]heptan-5-yl]-5,6-difluoro-8-(methylamino)-9H-pyrido[2,3-b]indol-3-yl]-1-methyl-4-oxo-1,8-naphthyridine-3-carboxylic acid;
6-[4-[(2R,3R)-2-amino-5-azaspiro[2.4]heptan-5-yl]-5-chloro-6-fluoro-8-(methylamino)-9H-pyrido[2,3-b]indol-3-yl]-1-methyl-4-oxo-1,8-naphthyridine-3-carboxylic acid;
6-[4-[(2S,3S)-2-amino-5-azaspiro[2.4]heptan-5-yl]-5-chloro-6-fluoro-8-(methylamino)-9H-pyrido[2,3-b]indol-3-yl]-1-methyl-4-oxo-1,8-naphthyridine-3-carboxylic acid;
6-[4-[(2S,3R)-2-amino-5-azaspiro[2.4]heptan-5-yl]-5-chloro-6-fluoro-8-(methylamino)-9H-pyrido[2,3-b]indol-3-yl]-1-methyl-4-oxo-1,8-naphthyridine-3-carboxylic acid;
6-[4-[(2R,3S)-2-amino-5-azaspiro [2 .4]heptan-5-yl]-5-chloro-6-fluoro-8-(methylamino)-9H-pyrido[2,3-b]indol-3-yl]-1-methyl-4-oxo-1,8-naphthyridine-3-carboxylic acid;
6-[4-[(25,3R)-2-amino-5-azaspiro[2.4]heptan-5-yl]-5-cyano-6-fluoro-8-(methylamino)-9H-pyrido[2,3-b]indol-3-yl]-1-methyl-4-oxo-1,8-naphthyridine-3-carboxylic acid;
6-[4-[(2R,3S)-2-amino-5-azaspiro[2.4]heptan-5-yl]-5-cyano-6-fluoro-8-(methylamino)-9H-pyrido[2,3-b]indol-3-yl]-1-methyl-4-oxo-1,8-naphthyridine-3-carboxylic acid;
6-[4-[(2S,3S)-2-amino-5-azaspiro[2.4]heptan-5-yl]-5-cyano-6-fluoro-8-(methylamino)-9H-pyrido[2,3-b]indol-3-yl]-1-methyl-4-oxo-1,8-naphthyridine-3-carboxylic acid;
6-[4-[(2R,3R)-2-amino-5-azaspiro[2.4]heptan-5-yl]-5-cyano-6-fluoro-8-(methylamino)-9H-pyrido[2,3-b]indol-3-yl]-1-methyl-4-oxo-1,8-naphthyridine-3-carboxylic acid;
6-[4-[(2R,3R)-2-amino-5-azaspiro[2.5]octan-5-yl]-5,6-difluoro-8-(methylamino)-9H-pyrido[2,3-b]indol-3-yl]-1-methyl-4-oxo-1,8-naphthyridine-3-carboxylic acid; and
6-[4-[(2S,3S)-2-amino-5-azaspiro[2.5]octan-5-yl]-5,6-difluoro-8-(methylamino)-9H-pyrido[2,3-b]indol-3-yl]-1-methyl-4-oxo-1,8-naphthyridine-3-carboxylic acid;
or a pharmaceutically acceptable salt thereof.

9. A process for the preparation of a compound of claim 1 comprising the reaction of compound of formula (Ii), 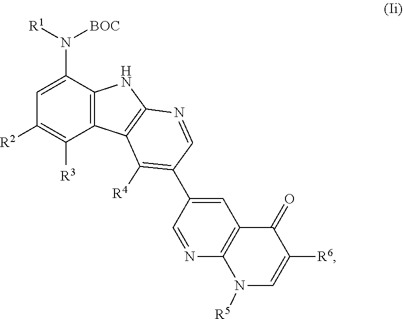
with an acid.

10. (canceled)

11. A pharmaceutical composition comprising a compound in accordance with claim 1 and a therapeutically inert carrier.

12-21. (canceled)

22. A method for the treatment of bacterial infection in a subject, which method comprises administering to said subject an effective amount of a compound of claim 1.

23. The method according to claim 22, wherein the bacterial infection is caused by Streptococcus pneumoniae, Streptococcus pyogenes, Enterococcus faecalis, Enterococcus faecium, Enterobacter spp. species, Proteus spp. species, Serratia marcescens, Staphylococcus aureus, Coag. Neg. Staphylococci, Haemophilus influenzae, Bacillus anthraces, Mycoplasma pneumoniae, Moraxella catarrhalis, Chlamydophila pneumoniae, Chlamydia trachomatis, Legionella pneumophila, Mycobacterium tuberculosis, Helicobacter pylori, Staphylococcus saprophyticus, Staphylococcus epidermidis, Francisella tularensis, Yersinia pestis, Clostridium difficile, Bacteroides spp. species Neisseria gonorrhoeae, Neisseria meningitidis, Burkholderia pseudomallei, Burkholderia mallei, Borrelia burgdorferi, Mycobacterium avium complex, Mycobacterium abscessus, Mycobacterium kansasii, E. Escherichia coli or Mycobacterium ulcerans.

24. The method according to claim 22, wherein the bacterial infection is caused by Acinetobacter baumannii, Klebsiella pneumoniae, Pseudomonas aeruginosa, Staphylococcus aureus or & Escherichia coli.

25. The compound according to claim 1, or a pharmaceutically acceptable salt thereof, wherein R¹ is methyl.

26. The compound according to claim 1, or a pharmaceutically acceptable salt thereof, wherein R² is fluoro.

27. The compound according to claim 1, or a pharmaceutically acceptable salt thereof, wherein R³ is chloro, fluoro or cyano.

28. The compound according to claim 1, or a pharmaceutically acceptable salt thereof, wherein R⁴ is pyrrolidinyl substituted once or twice by substituents each independently selected from amino, aminoC₁₋₆alkyl, aminoC₃₋₇cycloalkyl, haloC₁₋₆alkyl and halogen.

29. The compound according to claim 1, or a pharmaceutically acceptable salt thereof, wherein R⁵ is methyl or ethyl.

30. The compound of claim 1, or a pharmaceutically acceptable salt thereof, wherein
R¹ is methyl;
R² is fluoro;
R³ is fluoro;
R⁴ is selected from the group consisting of
1,3,4,5,6,6a-hexahydrocyclopenta[c]pyrrolyl substituted by amino;
2,3,3a,4,6,6a-hexahydropyrrolo[2,3-c]pyrrolyl substituted by amino;
3,3a,4,5,6,6a-hexahydro-1H-cyclopenta[c]pyrrolyl substituted by amino;
azabicyclo[3.1.0]hexanyl substituted by amino or aminoC₁₋₆alkyl;
azaspiro[2.4]heptanyl substituted by amino;
azaspiro[2.5]octanyl substituted by amino;
azaspiro[3.3]heptanyl substituted by amino;
piperidinyl substituted twice by aminoC₁₋₆alkyl and halogen; and
pyrrolidinyl substituted once or twice by substituents independently selected from amino, aminoC₁₋₆alkyl, aminoC₃₋₇cycloalkyl, haloC₁₋₆alkyl and halogen; and
R⁵ is methyl.
